SEQUENCE LISTING (203 sequences)

SEQ ID NO: 1-10 - skipped (10 entries)

SEQ ID NO: 11 - DNA, 1038 bp, Homo sapien
  CDS (1)...(930)
atggctaccgagagtactccctcagagatcatagaaagagaaagaaaaaagttgcttgaaatccttcaacatgatcctgattctatcttagacacgttaacttctcggaggctgatttctgaggaagagtatgagactctggagaatgttacagatctcctgaagaaaagtcggaagctgttaattttggtacagaaaaagggagaggcgacctgtcagcattttctcaagtgtttatttagtacttttccacagtcagctgccatttgcggcttaaggcatgaagttttaaaacatgagaatacagtacctcctcaatctatgggggcaagcagtaattcagaagatgctttttctcctggaataaaacagcctgaagcccctgagatcacagtgttcttcagtgagaaggaacacttggatttggaaacctctgagtttttcagggacaagaaaactagttatagggaaacagctttgtctgccaggaagaatgagaaggaatatgacacaccagaagtcacattatcatattcagttgagaaagttggatgtgaagttccagcaactattacatatataaaagatggacagagatatgaggagctagatgattctttatacttaggaaaagaggaatatctaggatctgttgacacccctgaagatgcagaagccactgtggaagaggaggtttatgatgacccagagcacgttggatatgatggtgaagaggacttcgagaattcagaaaccacagagttctctggtgaagaaccaagttatgagggatcagaaaccagcctttcattggaggaggaacaggagaaaagtatagaaggctggtctcgaactcatgggcttaagcgatcctcccacgttggcctcccaaagtgctgggattacaggcgtgagccaccctgcctggcctgaaaattctgcctcaaacatctcaaacatccatttatattttgtacaagaaagtaaataaaatttttctttttaacattaaaaaaaaaaaaaaaaaaaaaatctaga

SEQ ID NO: 12 - protein, 310 aa, Homo sapien
MATESTPSEIIERERKKLLEILQHDPDSILDTLTSRRLISEEEYETLENVTDLLKKSRKLLILVQKKGEATCQHFLKCLFSTFPQSAAICGLRHEVLKHENTVPPQSMGASSNSEDAFSPGIKQPEAPEITVFFSEKEHLDLETSEFFRDKKTSYRETALSARKNEKEYDTPEVTLSYSVEKVGCEVPATITYIKDGQRYEELDDSLYLGKEEYLGSVDTPEDAEATVEEEVYDDPEHVGYDGEEDFENSETTEFSGEEPSYEGSETSLSLEEEQEKSIEGWSRTHGLKRSSHVGLPKCWDYRREPPCLA

SEQ ID NO: 13-73 - skipped (61 entries)

SEQ ID NO: 74 - DNA, 19 bp, Artificial Sequence
  Primer
tacttactttgtcccttca

SEQ ID NO: 75 - DNA, 19 bp, Artificial Sequence
  Primer
tatttgtccccatctcgtc

SEQ ID NO: 76 - DNA, 28 bp, Artificial Sequence
  Primer
cggaattcatggctaccgagagtactcc

SEQ ID NO: 77 - DNA, 17 bp, Artificial Sequence
  Primer
gtaaaacgacggccagt

SEQ ID NO: 78 - DNA, 27 bp, Artificial Sequence
  Primer
gcagaagccactgtggaagaggaggtt

SEQ ID NO: 79 - DNA, 28 bp, Artificial Sequence
  Primer
atacgactcactatagggcgaattggcc

SEQ ID NO: 80 - skipped
  Primer

SEQ ID NO: 81 - skipped
  Primer

SEQ ID NO: 82 - DNA, 3030 bp, Homo sapien
  CDS (1)...(1680)
tgtgaaatgtgctcgcaggaggcttttcaggcacagaggagccagctggtcgagctgctggtctcagggtccctggaaggcttcgagagtgtcctggactggctgctgtcctgggaggtcctctcctgggaggactacgagggcttccacctcctgggccagcctctctcccacttggccaggcgccttctggacaccgtctggaataagggtacttgggcctgtcagaagctcatcgcggctgcccaagaagcccaggccgacagccagtcccccaagctgcatggctgctgggacccccactcgctccacccagcccgagacctgcagagtcaccggccagccattgtcaggaggctccacagccatgtggagaacatgctggacctggcatgggagcggggtttcgtcagccagtatgaatgtgatgaaatcaggttgccgatcttcacaccgtcccagagggcaagaaggctgcttgatcttgccacggtgaaagcgaatggattggctgccttccttctacaacatgttcaggaattaccagtcccattggccctgcctttggaagctgccacatgcaagaagtatatggccaagctgaggaccacggtgtctgctcagtctcgcttcctcagtacctatgatggagcagagacgctctgcctggaggacatatacacagagaatgtcctggaggtctgggcagatgtgggcatggctggacccccgcagaagagcccagccaccctgggcctggaggagctcttcagcacccctggccacctcaatgacgatgcggacactgtgctggtggtgggtgaggcgggcagtggcaagagcacgctcctgcagcggctgcacttgctgtgggctgcagggcaagacttccaggaatttctctttgtcttcccattcagctgccggcagctgcagtgcatggccaaaccactctctgtgcggactctactctttgagcactgctgttggcctgatgttggtcaagaagacatcttccagttactccttgaccaccctgaccgtgtcctgttaacctttgatggctttgacgagttcaagttcaggttcacggatcgtgaacgccactgctccccgaccgaccccacctctgtccagaccctgctcttcaaccttctgcagggcaacctgctgaagaatgcccgcaaggtggtgaccagccgtccggccgctgtgtcggcgttcctcaggaagtacatccgcaccgagttcaacctcaagggcttctctgaacagggcatcgagctgtacctgaggaagcgccatcatgagcccggggtggcggaccgcctcatccgcctgctccaagagacctcagccctgcacggtttgtgccacctgcctgtcttctcatggatggtgtccaaatgccaccaggaactgttgctgcaggagggggggtccccaaagaccactacagatatgtacctgctgattctgcagcattttctgctgcatgccacccccccagactcagcttcccaaggtctgggacccagtcttcttcggggccgcctccccaccctcctgcacctgggcagactggctctgtggggcctgggcatgtgctgctacgtgttctcagcccagcagctccaggcagcacaggtcagccctgatgacatttctcttggcttcctggtgcgtgccaaaggtgtcgtgccagggagtacggcgcccctggaattccttcacatcactttccagtgcttctttgccgcgttctacctggcactcagtgctgatgtgccaccagctttgctcagacacctcttcaattgtggcaggccaggcaactcaccaatggccaggctcctgcccacgatgtgcatccaggcctcggagggaaaggacagcagcgtggcagctttgctgcagaaggccgagccgcacaaccttcagatcacagcagccttcctggcagggctgttgtcccgggagcactggggcctgctggctgagtgccagacatctgagaaggccctgctccggcgccaggcctgtgcccgctggtgtctggcccgcagcctccgcaagcacttccactccatcccgccagctgcaccgggtgaggccaagagcgtgcatgccatgcccgggttcatctggctcatccggagcctgtacgagatgcaggaggagcggctggctcggaaggctgcacgtggcctgaatgttgggcacctcaagttgacattttgcagtgtgggccccactgagtgtgctgccctggcctttgtgctgcagcacctccggcggcccgtggccctgcagctggactacaactctgtgggtgacattggcgtggagcagctgctgccttgccttggtgtctgcaaggctctgtatttgcgcgataacaatatctcagaccgaggcatctgcaagctcattgaatgtgctcttcactgcgagcaattgcagaagttagcgctggggaataactacatcactgccgcgggagcccaagtgctggccgaggggctccgaggcaacacctccttgcagttcctgggattctggggcaacagagtgggtgacgagggggcccaggccctggctgaagccttgggtgatcaccagagcttgaggtggctcagcctggtggggaacaacattggcagtgtgggtgcccaagccttggcactgatgctggcaaagaacgtcatgctagaagaactctgcctggaggagaaccatctccaggatgaaggtgtatgttctctcgcagaaggactgaagaaaaattcaagtttgaaaatcctgaacataaaaattcatgcttcgggattcaacaaactcttggaaagcattttctgcatcctcctggttgtggaagcatttttcctgcagaaagttgtcaagattcttgaagaaatggtagtcagttggctagaggtcaggttgtccaataactgcatcacctacctaggggcagaagccctcctgcaggcccttgaaaggaatgacaccatcctggaagtctggtaa

SEQ ID NO: 83 - protein, 560 aa, Homo sapien
CEMCSQEAFQAQRSQLVELLVSGSLEGFESVLDWLLSWEVLSWEDYEGFHLLGQPLSHLARRLLDTVWNKGTWACQKLIAAAQEAQADSQSPKLHGCWDPHSLHPARDLQSHRPAIVRRLHSHVENMLDLAWERGFVSQYECDEIRLPIFTPSQRARRLLDLATVKANGLAAFLLQHVQELPVPLALPLEAATCKKYMAKLRTTVSAQSRFLSTYDGAETLCLEDIYTENVLEVWADVGMAGPPQKSPATLGLEELFSTPGHLNDDADTVLVVGEAGSGKSTLLQRLHLLWAAGQDFQEFLFVFPFSCRQLQCMAKPLSVRTLLFEHCCWPDVGQEDIFQLLLDHPDRVLLTFDGFDEFKFRFTDRERHCSPTDPTSVQTLLFNLLQGNLLKNARKVVTSRPAAVSAFLRKYIRTEFNLKGFSEQGIELYLRKRHHEPGVADRLIRLLQETSALHGLCHLPVFSWMVSKCHQELLLQEGGSPKTTTDMYLLILQHFLLHATPPDSASQGLGPSLLRGRLPTLLHLGRLALWGLGMCCYVFSAQQLQAAQVSPDDISLGFL

SEQ ID NO: 84 - DNA, 1107 bp, Homo sapien
attctttttttaacttttacttattcattaggatgatttcataatatatttcctggtttagaggaaacaggaacaatggctaccgagagtactccctcagagatcatagaaagagaaagaaaaaagttgcttgaaatccttcaacatgatcctgattctatcttagacacgttaacttctcggaggctgatttctgaggaagagtatgagactctggagaatgttacagatctcctgaagaaaagtcggaagctgttaattttggtacagaaaaagggagaggcgacctgtcagcattttctcaagtgtttatttagtacttttccacagtcagctgccatttgcggcttaaggcatgaagttttaaaacatgagaatacagtacctcctcaatctatgggggcaagcagtaattcagaagatgctttttctcctggaataaaacagcctgaagcccctgagatcacagtgttcttcagtgagaaggaacacttggatttggaaacctctgagtttttcagggacaagaaaactagttatagggaaacagctttgtctgccaggaagaatgagaaggaatatgacacaccagaagtcacattatcatattcagttgagaaagttggatgtgaagttccagcaactattacatatataaaagatggacagagatatgaggagctagatgattctttatacttaggaaaagaggaatatctaggatctgttgacacccctgaagatgcagaagccactgtggaagaggaggtttatgatgacccagagcacgttggatatgatggtgaagaggacttcgagaattcagaaaccacagagttctctggtgaagaaccaagttatgagggatcagaaaccagcctttcattggaggaggaacaggagaaaagtatagaaggctggtctcgaactcatgggcttaagcgatcctcccacgttggcctcccaaagtgctgggattacaggcgtgagccaccctgcctggcctgaaaattctgcctcaaacatctcaaacatccatttatattttgtacaagaaagtaaataaaatttttctttttaacattaaaaaaaaaaaaaaaaaaaaaa

SEQ ID NO: 85 - DNA, 510 bp, Homo sapien
  CDS (15)...(305)
aggagagaaaagccatggccgacaaggtcctgaaggagaagagaaagctgtttatccattccatgggtgaaggtacaataaatggcttactggatgaattattacagacaagggtgctgaaccaggaagagatggagaaagtaaaacgtgaaaatgctacagttatggataagacccgagctttgattgactccgttattccgaaaggggcacaggcatgccaaatttgcatcacatacatttgtgaagaagacagttacctggcagagacgctgggactctcagcaggtccgatacctggaaattagcttagcttagtacacaagactcccaattactattttcttccttcccagctcttcaggcagtgcaggacaacccagctatgcccacatgctcaagcccagaaggcagaatcaagctttgctttctagaagacgctcaaaggatatggaaacaaaagttgcagaggtgccatgttcagaatacaataataaagtggagtaaaga

SEQ ID NO: 86 - protein, 97 aa, Homo sapien
MADKVLKEKRKLFIHSMGEGTINGLLDELLQTRVLNQEEMEKVKRENATVMDKTRALIDSVIPKGAQACQICITYICEEDSYLAETLGLSAGPIPGN

SEQ ID NO: 87 - protein, 97 aa, Homo sapien
MADKVLKEKRKLFIRSMGEGTINGLLDELLQTRVLNKEEMEKVKRENATVMDKTRALIDSVIPKGAQACQICITYICEEDSYLAGTLGLSADQTSGN

SEQ ID NO: 88 - skipped

SEQ ID NO: 89 - DNA, 321 bp, Homo sapien
  CDS (1)...(318)
atgatcctcctgaagaagagacggctgcttatcaattcattgggtgaaggtacaataaatggcttactggatgaattattggagacaaatgtgctgagccaggaagacacagagatagtaaaatgtgaaaatgttacagttatcgataaggcccgagatttgcttgactctgttattcggaaaggggcaggggcatgtgaaatttgcatcacatacatttgtgaagaagacaggtacctggcagggacgctgggactctcagcaggaaatgactacagagctggaggcatttgctcaccgcccagagcacaagacctctga

SEQ ID NO: 90 - protein, 106 aa, Homo sapien
MILLKKRRLLINSLGEGTINGLLDELLETNVLSQEDTEIVKCENVTVIDKARDLLDSVIRKGAGACEICITYICEEDRYLAGTLGLSAGNDYRAGGICSPPRAQDL

SEQ ID NO: 91 - protein, 108 aa, Homo sapien
MADKVLLEKRKLLINSLGEGTINGLLDELLETNVLSQEDEIVKRENATVIDKARALLDSVIRKGAGACEICITYICEEDSYLAGTLGLSAGNAVQAGGACSTSSGQDL

SEQ ID NO: 92-95 - skipped (4 entries)

SEQ ID NO: 96 - DNA, 3396 bp, Homo sapien
  CDS (277)...(3348)
cgcccgggcaggtgtttatactccggagggtgtccccgtgcgtcatcggtggagtggaccaaaactggtgatctgtttgccctgtgtgaccttgcccagaaccctgctgactgagagaacacatctgctggaagtcctctgggattcaaggtacagggaatgaagagtagttttacagaaaaaagaggacaatattgggatcacctttgacctttccatttggaaataatattttctattgtgttatagaaaggtgggaagctttcatccagaacaatgaatttcataaaggacaatagccgagcccttattcaaagaatgggaatgactgttataaagcaaatcacagatgacctatttgtatggaatgttctgaatcgcgaagaagtaaacatcatttgctgcgagaaggtggagcaggatgctgctagagggatcattcacatgattttgaaaaagggttcagagtcctgtaacctctttcttaaatcccttaaggagtggaactatcctctatttcaggacttgaatggacaaagtctttttcatcagacatcagaaggagacttggacgatttggctcaggatttaaaggacttgtaccataccccatcttttctgaacttttatccccttggtgaagatattgacattatttttaacttgaaaagcaccttcacagaacctatcctgtggaggaaggaccaacaccatcaccgcgtggagcagctgaccctgaatggcctcctgcaggctcttcagagcccctgcatcattgaaggggaatctggcaaaggcaagtccactctgctgcagcgcattgccatgctctggggctccggaaagtgcaaggctctgaccaagttcaaattcgtcttcttcctccgtctcagcagggcccagggtggactttttgaaaccctctgtgatcaactcctggatatacctggcacaatcaggaagcagacattcatggccatgctgctgaagctgcggcagagggttcttttccttcttgatggctacaatgaattcaagccccagaactgcccagaaatcgaagccctgataaaggaaaaccaccgcttcaagaacatggtcatcgtcaccactaccactgagtgcctgaggcacatacggcagtttggtgccctgactgctgaggtgggggatatgacagaagacagcgcccaggctctcatccgagaagtgctgatcaaggagcttgctgaaggcttgttgctccaaattcagaaatccaggtgcttgaggaatctcatgaagacccctctctttgtggtcatcacttgtgcaatccagatgggtgaaagtgagttccactctcacacacaaacaacgctgttccataccttctatgatctgttgatacagaaaaacaaacacaaacataaaggtgtggctgcaagtgacttcattcggagcctggaccaccgtggagacctagctctggagggtgtgttctcccacaagtttgatttcgaactgcaggatgtgtccagcgtgaatgaggatgtcctgctgacaactgggctcctctgtaaatatacagctcaaaggttcaagccaaagtataaattctttcacaagtcattccaggagtacacagcaggacgaagactcagcagtttattgacgtctcatgagccagaggaggtgaccaaggggaatggttacttgcagaaaatggtttccatttcggacattacatccacttatagcagcctgctccggtacacctgtgggtcatctgtggaagccaccagggctgttatgaagcacctcgcagcagtgtatcaacacggctgccttctcggactttccatcgccaagaggcctctctggagacaggaatctttgcaaagtgtgaaaaacaccactgagcaagaaattctgaaagccataaacatcaattcctttgtagagtgtggcatccatttatatcaagagagtacatccaaatcagccctgagccaagaatttgaagctttctttcaaggtaaaagcttatatatcaactcagggaacatccccgattacttatttgacttctttgaacatttgcccaattgtgcaagtgccctggacttcattaaactggacttttatgggggagctatggcttcatgggaaaaggctgcagaagacacaggtggaatccacatggaagaggccccagaaacctacattcccagcagggctgtatctttgttcttcaactggaagcaggaattcaggactctggaggtcacactccgggatttcagcaagttgaataagcaagatatcagatatctggggaaaatattcagctctgccacaagcctcaggctgcaaataaagagatgtgctggtgtggctggaagcctcagtttggtcctcagcacctgtaagaacatttattctctcatggtggaagccagtcccctcaccatagaagatgagaggcacatcacatctgtaacaaacctgaaaaccttgagtattcatgacctacagaatcaacggctgccgggtggtctgactgacagcttgggtaacttgaagaaccttacaaagctcataatggataacataaagatgaatgaagaagatgctataaaactagctgaaggcctgaaaaacctgaagaagatgtgtttatttcatttgacccacttgtctgacattggagagggaatggattacatagtcaagtctctgtcaagtgaaccctgtgaccttgaagaaattcaattagtctcctgctgcttgtctgcaaatgcagtgaaaatcctagctcagaatcttcacaatttggtcaaactgagcattcttgatttatcagaaaattacctggaaaaagatggaaatgaagctcttcatgaactgatcgacaggatgaacgtgctagaacagctcaccgcactgatgctgccctggggctgtgacgtgcaaggcagcctgagcagcctgttgaaacatttggaggaggtcccacaactcgtcaagcttgggttgaaaaactggagactcacagatacagagattagaattttaggtgcattttttggaaagaaccctctgaaaaacttccagcagttgaatttggcgggaaatcgtgtgagcagtgatggatggcttgccttcatgggtgtatttgagaatcttaagcaattagtgttttttgactttagtactaaagaatttctacctgatccagcattagtcagaaaacttagccaagtgttatccaagttaacttttctgcaagaagctaggcttgttgggtggcaatttgatgatgatgatctcagtgttattacaggtgcttttaaactagtaactgcttaaataaagtgtactcgaagccaaaaaaaaaaaaaaaaaaaaaaaaaa

SEQ ID NO: 97 - protein, 1024 aa, Homo sapien
MNFIKDNSRALIQRMGMTVIKQITDDLFVWNVLNREEVNIICCEKVEQDAARGIIHMILKKGSESCNLFLKSLKEWNYPLFQDLNGQSLFHQTSEGDLDDLAQDLKDLYHTPSFLNFYPLGEDIDIIFNLKSTFTEPILWRKDQHHHRVEQLTLNGLLQALQSPCIIEGESGKGKSTLLQRIAMLWGSGKCKALTKFKFVFFLRLSRAQGGLFETLCDQLLDIPGTIRKQTFMAMLLKLRQRVLFLLDGYNEFKPQNCPEIEALIKENHRFKNMVIVTTTTECLRHIRQFGALTAEVGDMTEDSAQALIREVLIKELAEGLLLQIQKSRCLRNLMKTPLFVVITCAIQMGESEFHSHTQTTLFHTFYDLLIQKNKHKHKGVAASDFIRSLDHRGDLALEGVFSHKFDFELQDVSSVNEDVLLTTGLLCKYTAQRFKPKYKFFHKSFQEYTAGRRLSSLLTSHEPEEVTKGNGYLQKMVSISDITSTYSSLLRYTCGSSVEATRAVMKHLAAVYQHGCLLGLSIAKRPLWRQESLQSVKNTTEQEILKAININSFVECGIHLYQESTSKSALSQEFEAFFQGKSLYINSGNIPDYLFDFFEHLPNCASALDFIKLDFYGGAMASWEKAAEDTGGIHMEEAPETYIPSRAVSLFFNWKQEFRTLEVTLRDFSKLNKQDIRYLGKIFSSATSLRLQIKRCAGVAGSLSLVLSTCKNIYSLMVEASPLTIEDERHITSVTNLKTLSIHDLQNQRLPGGLTDSLGNLKNLTKLIMDNIKMNEEDAIKLAEGLKNLKKMCLFHLTHLSDIGEGMDYIVKSLSSEPCDLEEIQLVSCCLSANAVKILAQNLHNLVKLSILDLSENYLEKDGNEALHELIDRMNVLEQLTALMLPWGCDVQGSLSSLLKHLEEVPQLVKLGLKNWRLTDTEIRILGAFFGKNPLKNFQQLNLAGNRVSSDGWLAFMGVFENLKQLVFFDFSTKEFLPDPALVRKLSQVLSKLTFLQEARLVGWQFDDDDLSVITGAFKLVTA

SEQ ID NO: 98 - DNA, 1395 bp, Homo sapien
  CDS (277)...(1353)
cgcccgggcaggtgtttatactccggagggtgtccccgtgcgtcatcggtggagtggaccaaaactggtgatctgtttgccctgtgtgaccttgcccagaaccctgctgactgagagaacacatctgctggaagtcctctgggattcaaggtacagggaatgaagagtagttttacagaaaaaagaggacaatattgggatcacctttgacctttccatttggaaataatattttctattgtgttatagaaaggtgggaagctttcatccagaacaatgaatttcataaaggacaatagccgagcccttattcaaagaatgggaatgactgttataaagcaaatcacagatgacctatttgtatggaatgttctgaatcgcgaagaagtaaacatcatttgctgcgagaaggtggagcaggatgctgctagagggatcattcacatgattttgaaaaagggttcagagtcctgtaacctctttcttaaatcccttaaggagtggaactatcctctatttcaggacttgaatggacaaagtggtctgactgacagcttgggtaacttgaagaaccttacaaagctcataatggataacataaagatgaatgaagaagatgctataaaactagctgaaggcctgaaaaacctgaagaagatgtgtttatttcatttgacccacttgtctgacattggagagggaatggattacatagtcaagtctctgtcaagtgaaccctgtgaccttgaagaaattcaattagtctcctgctgcttgtctgcaaatgcagtgaaaatcctagctcagaatcttcacaatttggtcaaactgagcattcttgatttatcagaaaattacctggaaaaagatggaaatgaagctcttcatgaactgatcgacaggatgaacgtgctagaacagctcaccgcactgatgctgccctggggctgtgacgtgcaaggcagcctgagcagcctgttgaaacatttggaggaggtcccacaactcgtcaagcttgggttgaaaaactggagactcacagatacagagattagaattttaggtgcattttttggaaagaaccctctgaaaaacttccagcagttgaatttggcgggaaatcgtgtgagcagtgatggatggcttgccttcatgggtgtatttgagaatcttaagcaattagtgttttttgactttagtactaaagaatttctacctgatccagcattagtcagaaaacttagccaagtgttatccaagttaacttttctgcaagaagctaggcttgttgggtggcaatttgatgatgatgatctcagtgttattacaggtgcttttaaactagtaactgcttaaataaagtgtactcgaagcaaaaaaaaaaaaaaaaaaaaa

SEQ ID NO: 99 - protein, 359 aa, Homo sapien
MNFIKDNSRALIQRMGMTVIKQITDDLFVWNVLNREEVNIICCEKVEQDAARGIIHMILKKGSESCNLFLKSLKEWNYPLFQDLNGQSGLTDSLGNLKNLTKLIMDNIKMNEEDAIKLAEGLKNLKKMCLFHLTHLSDIGEGMDYIVKSLSSEPCDLEEIQLVSCCLSANAVKILAQNLHNLVKLSILDLSENYLEKDGNEALHELIDRMNVLEQLTALMLPWGCDVQGSLSSLLKHLEEVPQLVKLGLKNWRLTDTEIRILGAFFGKNPLKNFQQLNLAGNRVSSDGWLAFMGVFENLKQLVFFDFSTKEFLPDPALVRKLSQVLSKLTFLQEARLVGWQFDDDDLSVITGAFKLVTA

SEQ ID NO: 100 - DNA, 578 bp, Homo sapien
  CDS (277)...(552)
cgcccgggcaggtgtttatactccggagggtgtccccgtgcgtcatcggtggagtggaccaaaactggtgatctgtttgccctgtgtgaccttgcccagaaccctgctgactgagagaacacatctgctggaagtcctctgggattcaaggtacagggaatgaagagtagttttacagaaaaaagaggacaatattgggatcacctttgacctttccatttggaaataatattttctattgtgttatagaaaggtgggaagctttcatccagaacaatgaatttcataaaggacaatagccgagcccttattcaaagaatgggaatgactgttataaagcaaatcacagatgacctatttgtatggaatgttctgaatcgcgaagaagtaaacatcatttgctgcgagaaggtggagcaggatgctgctagagggatcattcacatgattttgaaaaagggttcagagtcctgtaacctctttcttaaatcccttaaggagtggaactatcctctatttcaggacttgaatggacaaagtcttttaacagcttagaaagtacagtagacatactgggg

SEQ ID NO: 101 - protein, 92 aa, Homo sapien
MNFIKDNSRALIQRMGMTVIKQITDDLFVWNVLNREEVNIICCEKVEQDAARGIIHMILKKGSESCNLFLKSLKEWNYPLFQDLNGQSLLTA

SEQ ID NO: 102 - DNA, 768 bp, Homo sapien
  CDS (277)...(744)
cgcccgggcaggtgtttatactccggagggtgtccccgtgcgtcatcggtggagtggaccaaaactggtgatctgtttgccctgtgtgaccttgcccagaaccctgctgactgagagaacacatctgctggaagtcctctgggattcaaggtacagggaatgaagagtagttttacagaaaaaagaggacaatattgggatcacctttgacctttccatttggaaataatattttctattgtgttatagaaaggtgggaagctttcatccagaacaatgaatttcataaaggacaatagccgagcccttattcaaagaatgggaatgactgttataaagcaaatcacagatgacctatttgtatggaatgttctgaatcgcgaagaagtaaacatcatttgctgcgagaaggtggagcaggatgctgctagagggatcattcacatgattttgaaaaagggttcagagtcctgtaacctctttcttaaatcccttaaggagtggaactatcctctatttcaggacttgaatggacaaagtctttttcatcagacatcagaaggagacttggacgatttggctcaggatttaaaggacttgtaccataccccatcttttctgaacttttatccccttggtgaagatattgacattatttttaacttgaaaagcaccttcacagaacctgtcctgtggaggaaggaccaacaccatcaccgcgtggagcagctgaccctagttttatagcatcttctacctgcccgggcg

SEQ ID NO: 103 - protein, 156 aa, Homo sapien
MNFIKDNSRALIQRMGMTVIKQITDDLFVWNVLNREEVNIICCEKVEQDAARGIIHMILKKGSESCNLFLKSLKEWNYPLFQDLNGQSLFHQTSEGDLDDLAQDLKDLYHTPSFLNFYPLGEDIDIIFNLKSTFTEPVLWRKDQHHHRVEQLTLVL

SEQ ID NO: 104 - DNA, 24 bp, Artificial Sequence
  Primer
aagaagagacggctgcttatcaat

SEQ ID NO: 105 - DNA, 24 bp, Artificial Sequence
  Primer
ccacagcaggcctcgaagatgatc

SEQ ID NO: 106 - DNA, 20 bp, Artificial Sequence
  Primer
atgatcctcctgaagaagag

SEQ ID NO: 107 - protein, 1009 aa, Homo sapien
CEMCSQEAFQAQRSQLVELLVSGSLEGFESVLDWLLSWEVLSWEDYEGFHLLGQPLSHLARRLLDTVWNKGTWACQKLIAAAQEAQADSQSPKLHGCWDPHSLHPARDLQSHRPAIVRRLHSHVENMLDLAWERGFVSQYECDEIRLPIFTPSQRARRLLDLATVKANGLAAFLLQHVQELPVPLALPLEAATCKKYMAKLRTTVSAQSRFLSTYDGAETLCLEDIYTENVLEVWADVGMAGPPQKSPATLGLEELFSTPGHLNDDADTVLVVGEAGSGKSTLLQRLHLLWAAGQDFQEFLFVFPFSCRQLQCMAKPLSVRTLLFEHCCWPDVGQEDIFQLLLDHPDRVLLTFDGFDEFKFRFTDRERHCSPTDPTSVQTLLFNLLQGNLLKNARKVVTSRPAAVSAFLRKYIRTEFNLKGFSEQGIELYLRKRHHEPGVADRLIRLLQETSALHGLCHLPVFSWMVSKCHQELLLQEGGSPKTTTDMYLLILQHFLLHATPPDSASQGLGPSLLRGRLPTLLHLGRLALWGLGMCCYVFSAQQLQAAQVSPDDISLGFLVRAKGVVPGSTAPLEFLHITFQCFFAAFYLALSADVPPALLRHLFNCGRPGNSPMARLLPTMCIQASEGKDSSVAALLQKAEPHNLQITAAFLAGLLSREHWGLLAECQTSEKALLRRQACARWCLARSLRKHFHSIPPAAPGEAKSVHAMPGFIWLIRSLYEMQEERLARKAARGLNVGHLKLTFCSVGPTECAALAFVLQHLRRPVALQLDYNSVGDIGVEQLLPCLGVCKALYLRDNNISDRGICKLIECALHCEQLQKLALGNNYITAAGAQVLAEGLRGNTSLQFLGFWGNRVGDEGAQALAEALGDHQSLRWLSLVGNNIGSVGAQALALMLAKNVMLEELCLEENHLQDEGVCSLAEGLKKNSSLKILNIKIHASGFNKLLESIFCILLVVEAFFLQKVVKILEEMVVSWLEVRLSNNCITYLGAEALLQALERNDTILEVW

SEQ ID NO: 108-144 - skipped (37 entries)

SEQ ID NO: 145 - DNA, 30 bp, Artificial Sequence
  primer
ccagaattcatggccgacaaggtcctgaag

SEQ ID NO: 146 - DNA, 30 bp, Artificial Sequence
  primer
ccactcgagctaatttccaggtatcggacc

SEQ ID NO: 147 - DNA, 20 bp, Artificial Sequence
  primer
gaagacagttacctggcaga

SEQ ID NO: 148 - DNA, 21 bp, Artificial Sequence
  primer
ttgtattctgaacatggcacc

SEQ ID NO: 149 - DNA, 36 bp, Artificial Sequence
  primer
gatcatcatccaggccgcccgtggtgacagccctgg

SEQ ID NO: 150 - DNA, 36 bp, Artificial Sequence
  primer
ccagggctgtcaccacgggcggcctggatgatgatc

SEQ ID NO: 151 - DNA, 26 bp, Artificial Sequence
  primer
cggaattcatggccgacaaggtcctg

SEQ ID NO: 152 - DNA, 38 bp, Artificial Sequence
  primer
cgctcgagttagtcttgcatattaaggtaatttccaga

SEQ ID NO: 153 - DNA, 23 bp, Artificial Sequence
  primer
catgtgaatgatccctctagcag

SEQ ID NO: 154 - DNA, 21 bp, Artificial Sequence
  primer
gggctcggctatcgtgctcta

SEQ ID NO: 155 - DNA, 21 bp, Artificial Sequence
  primer
acgatagccgagcccttattc

SEQ ID NO: 156 - DNA, 21 bp, Artificial Sequence
  primer
gtatggaatgttctgaatcgc

SEQ ID NO: 157 - DNA, 33 bp, Artificial Sequence
  primer
cccggatccatgaatttcataaaggacaatagc

SEQ ID NO: 158 - DNA, 30 bp, Artificial Sequence
  primer
cccttcgaacaagtcctgaaatagaggata

SEQ ID NO: 159 - DNA, 24 bp, Artificial Sequence
  primer
ggtggagcaggatgctgctagagg

SEQ ID NO: 160 - DNA, 29 bp, Artificial Sequence
  primer
cacagtggtccaggctccgaatgaagtca

SEQ ID NO: 161 - DNA, 25 bp, Artificial Sequence
  primer
catcatttgctgcgagaaggtggag

SEQ ID NO: 162 - DNA, 25 bp, Artificial Sequence
  primer
ttaacttggataacacttggctaag

SEQ ID NO: 163 - DNA, 23 bp, Artificial Sequence
  primer
gtaaacatcatttgctgcgagaa

SEQ ID NO: 164 - DNA, 23 bp, Artificial Sequence
  primer
cccgggcaggtagaagatgctat

SEQ ID NO: 165 - DNA, 25 bp, Artificial Sequence
  primer
aatttcataaaggacaatagccgag

SEQ ID NO: 166 - DNA, 25 bp, Artificial Sequence
  primer
tgtctactgtactttctaagctgtt

SEQ ID NO: 167 - DNA, 225 bp, Homo sapiens
  CDS (1)...(225)
gagagtactccctcagagatcatagaaagagaaagaaaaaagttgcttgaaatccttcaacatgatcctgattctatcttagacacgttaacttctcggaggctgatttctgaggaagagtatgagactctggagaatgttacagatctcctgaagaaaagtcggaagctgttaattttggtacagaaaaagggagaggcgacctgtcagcattttctcaagtgt

SEQ ID NO: 168 - protein, 75 aa, Homo sapiens
ESTPSEIIERERKKLLEILQHDPDSILDTLTSRRLISEEEYETLENVTDLLKKSRKLLILVQKKGEATCQHFLKC

SEQ ID NO: 169 - DNA, 228 bp, Homo sapiens
  CDS (1)...(228)
atgtgctcgcaggaggcttttcaggcacagaggagccagctggtcgagctgctggtctcagggtccctggaaggcttcgagagtgtcctggactggctgctgtcctgggaggtcctctcctgggaggactacgagggcttccacctcctgggccagcctctctcccacttggccaggcgccttctggacaccgtctggaataagggtacttgggcctgtcagaagctc

SEQ ID NO: 170 - protein, 76 aa, Homo sapiens
MCSQEAFQAQRSQLVELLVSGSLEGFESVLDWLLSWEVLSWEDYEGFHLLGQPLSHLARRLLDTVWNKGTWACQKL

SEQ ID NO: 171 - DNA, 243 bp, Homo sapiens
  CDS (1)...(243)
ccagcccgagacctgcagagtcaccggccagccattgtcaggaggctccacagccatgtggagaacatgctggacctggcatgggagcggggtttcgtcagccagtatgaatgtgatgaaatcaggttgccgatcttcacaccgtcccagagggcaagaaggctgcttgatcttgccacggtgaaagcgaatggattggctgccttccttctacaacatgttcaggaattaccagtcccattg

SEQ ID NO: 172 - protein, 81 aa, Homo sapiens
PARDLQSHRPAIVRRLHSHVENMLDLAWERGFVSQYECDEIRLPIFTPSQRARRLLDLATVKANGLAAFLLQHVQELPVPL

SEQ ID NO: 173 - DNA, 888 bp, Homo sapiens
  CDS (1)...(888)
gacgatgcggacactgtgctggtggtgggtgaggcgggcagtggcaagagcacgctcctgcagcggctgcacttgctgtgggctgcagggcaagacttccaggaatttctctttgtcttcccattcagctgccggcagctgcagtgcatggccaaaccactctctgtgcggactctactctttgagcactgctgttggcctgatgttggtcaagaagacatcttccagttactccttgaccaccctgaccgtgtcctgttaacctttgatggctttgacgagttcaagttcaggttcacggatcgtgaacgccactgctccccgaccgaccccacctctgtccagaccctgctcttcaaccttctgcagggcaacctgctgaagaatgcccgcaaggtggtgaccagccgtccggccgctgtgtcggcgttcctcaggaagtacatccgcaccgagttcaacctcaagggcttctctgaacagggcatcgagctgtacctgaggaagcgccatcatgagcccggggtggcggaccgcctcatccgcctgctccaagagacctcagccctgcacggtttgtgccacctgcctgtcttctcatggatggtgtccaaatgccaccaggaactgttgctgcaggagggggggtccccaaagaccactacagatatgtacctgctgattctgcagcattttctgctgcatgccacccccccagactcagcttcccaaggtctgggacccagtcttcttcggggccgcctccccaccctcctgcacctgggcagactggctctgtggggcctgggcatgtgctgctacgtgttctcagcccagcagctccaggcagcacaggtcagccctgatgacatttctcttggcttcctg

SEQ ID NO: 174 - protein, 296 aa, Homo sapiens
DDADTVLVVGEAGSGKSTLLQRLHLLWAAGQDFQEFLFVFPFSCRQLQCMAKPLSVRTLLFEHCCWPDVGQEDIFQLLLDHPDRVLLTFDGFDEFKFRFTDRERHCSPTDPTSVQTLLFNLLQGNLLKNARKVVTSRPAAVSAFLRKYIRTEFNLKGFSEQGIELYLRKRHHEPGVADRLIRLLQETSALHGLCHLPVFSWMVSKCHQELLLQEGGSPKTTTDMYLLILQHFLLHATPPDSASQGLGPSLLRGRLPTLLHLGRLALWGLGMCCYVFSAQQLQAAQVSPDDISLGFL

SEQ ID NO: 175 - DNA, 1209 bp, Homo sapiens
  CDS (1)...(1209)
gagcccggggtggcggaccgcctcatccgcctgctccaagagacctcagccctgcacggtttgtgccacctgcctgtcttctcatggatggtgtccaaatgccaccaggaactgttgctgcaggagggggggtccccaaagaccactacagatatgtacctgctgattctgcagcattttctgctgcatgccacccccccagactcagcttcccaaggtctgggacccagtcttcttcggggccgcctccccaccctcctgcacctgggcagactggctctgtggggcctgggcatgtgctgctacgtgttctcagcccagcagctccaggcagcacaggtcagccctgatgacatttctcttggcttcctggtgcgtgccaaaggtgtcgtgccagggagtacggcgcccctggaattccttcacatcactttccagtgcttctttgccgcgttctacctggcactcagtgctgatgtgccaccagctttgctcagacacctcttcaattgtggcaggccaggcaactcaccaatggccaggctcctgcccacgatgtgcatccaggcctcggagggaaaggacagcagcgtggcagctttgctgcagaaggccgagccgcacaaccttcagatcacagcagccttcctggcagggctgttgtcccgggagcactggggcctgctggctgagtgccagacatctgagaaggccctgctccggcgccaggcctgtgcccgctggtgtctggcccgcagcctccgcaagcacttccactccatcccgccagctgcaccgggtgaggccaagagcgtgcatgccatgcccgggttcatctggctcatccggagcctgtacgagatgcaggaggagcggctggctcggaaggctgcacgtggcctgaatgttgggcacctcaagttgacattttgcagtgtgggccccactgagtgtgctgccctggcctttgtgctgcagcacctccggcggcccgtggccctgcagctggactacaactctgtgggtgacattggcgtggagcagctgctgccttgccttggtgtctgcaaggctctgtatttgcgcgataacaatatctcagaccgaggcatctgcaagctcattgaatgtgctcttcactgcgagcaattgcagaagttagcgctggggaataactacatcactgccgcgggagcccaagtgctggcc

SEQ ID NO: 176 - protein, 403 aa, Homo sapiens
EPGVADRLIRLLQETSALHGLCHLPVFSWMVSKCHQELLLQEGGSPKTTTDMYLLILQHFLLHATPPDSASQGLGPSLLRGRLPTLLHLGRLALWGLGMCCYVFSAQQLQAAQVSPDDISLGFLVRAKGVVPGSTAPLEFLHITFQCFFAAFYLALSADVPPALLRHLFNCGRPGNSPMARLLPTMCIQASEGKDSSVAALLQKAEPHNLQITAAFLAGLLSREHWGLLAECQTSEKALLRRQACARWCLARSLRKHFHSIPPAAPGEAKSVHAMPGFIWLIRSLYEMQEERLARKAARGLNVGHLKLTFCSVGPTECAALAFVLQHLRRPVALQLDYNSVGDIGVEQLLPCLGVCKALYLRDNNISDRGICKLIECALHCEQLQKLALGNNYITAAGAQVLA

SEQ ID NO: 177 - DNA, 261 bp, Homo sapiens
  CDS (1)...(261)
atgaatttcataaaggacaatagccgagcccttattcaaagaatgggaatgactgttataaagcaaatcacagatgacctatttgtatggaatgttctgaatcgcgaagaagtaaacatcatttgctgcgagaaggtggagcaggatgctgctagagggatcattcacatgattttgaaaaagggttcagagtcctgtaacctctttcttaaatcccttaaggagtggaactatcctctatttcaggacttgaatggacaa

SEQ ID NO: 178 - protein, 87 aa, Homo sapiens
MNFIKDNSRALIQRMGMTVIKQITDDLFVWNVLNREEVNIICCEKVEQDAARGIIHMILKKGSESCNLFLKSLKEWNYPLFQDLNGQ

SEQ ID NO: 179 - DNA, 891 bp, Homo sapiens
  CDS (1)...(891)
cttcagagcccctgcatcattgaaggggaatctggcaaaggcaagtccactctgctgcagcgcattgccatgctctggggctccggaaagtgcaaggctctgaccaagttcaaattcgtcttcttcctccgtctcagcagggcccagggtggactttttgaaaccctctgtgatcaactcctggatatacctggcacaatcaggaagcagacattcatggccatgctgctgaagctgcggcagagggttcttttccttcttgatggctacaatgaattcaagccccagaactgcccagaaatcgaagccctgataaaggaaaaccaccgcttcaagaacatggtcatcgtcaccactaccactgagtgcctgaggcacatacggcagtttggtgccctgactgctgaggtgggggatatgacagaagacagcgcccaggctctcatccgagaagtgctgatcaaggagcttgctgaaggcttgttgctccaaattcagaaatccaggtgcttgaggaatctcatgaagacccctctctttgtggtcatcacttgtgcaatccagatgggtgaaagtgagttccactctcacacacaaacaacgctgttccataccttctatgatctgttgatacagaaaaacaaacacaaacataaaggtgtggctgcaagtgacttcattcggagcctggaccaccgtggagacctagctctggagggtgtgttctcccacaagtttgatttcgaactgcaggatgtgtccagcgtgaatgaggatgtcctgctgacaactgggctcctctgtaaatatacagctcaaaggttcaagccaaagtataaattctttcacaagtcattccaggagtacacagcaggacgaagactcagcagt

SEQ ID NO: 180 - protein, 297 aa, Homo sapiens
LQSPCIIEGESGKGKSTLLQRIAMLWGSGKCKALTKFKFVFFLRLSRAQGGLFETLCDQLLDIPGTIRKQTFMAMLLKLRQRVLFLLDGYNEFKPQNCPEIEALIKENHRFKNMVIVTTTTECLRHIRQFGALTAEVGDMTEDSAQALIREVLIKELAEGLLLQIQKSRCLRNLMKTPLFVVITCAIQMGESEFHSHTQTTLFHTFYDLLIQKNKHKHKGVAASDFIRSLDHRGDLALEGVFSHKFDFELQDVSSVNEDVLLTTGLLCKYTAQRFKPKYKFFHKSFQEYTAGRRLSS

SEQ ID NO: 181 - DNA, 618 bp, Homo sapiens
  CDS (1)...(618)
ggtaacttgaagaaccttacaaagctcataatggataacataaagatgaatgaagaagatgctataaaactagctgaaggcctgaaaaacctgaagaagatgtgtttatttcatttgacccacttgtctgacattggagagggaatggattacatagtcaagtctctgtcaagtgaaccctgtgaccttgaagaaattcaattagtctcctgctgcttgtctgcaaatgcagtgaaaatcctagctcagaatcttcacaatttggtcaaactgagcattcttgatttatcagaaaattacctggaaaaagatggaaatgaagctcttcatgaactgatcgacaggatgaacgtgctagaacagctcaccgcactgatgctgccctggggctgtgacgtgcaaggcagcctgagcagcctgttgaaacatttggaggaggtcccacaactcgtcaagcttgggttgaaaaactggagactcacagatacagagattagaattttaggtgcattttttggaaagaaccctctgaaaaacttccagcagttgaatttggcgggaaatcgtgtgagcagtgatggatggcttgccttcatgggtgtatttgagaatcttaag

SEQ ID NO: 182 - protein, 206 aa, Homo sapiens
GNLKNLTKLIMDNIKMNEEDAIKLAEGLKNLKKMCLFHLTHLSDIGEGMDYIVKSLSSEPCDLEEIQLVSCCLSANAVKILAQNLHNLVKLSILDLSENYLEKDGNEALHELIDRMNVLEQLTALMLPWGCDVQGSLSSLLKHLEEVPQLVKLGLKNWRLTDTEIRILGAFFGKNPLKNFQQLNLAGNRVSSDGWLAFMGVFENLK

SEQ ID NO: 183 - DNA, 165 bp, Homo sapiens
  CDS (1)...(165)
acctacattcccagcagggctgtatctttgttcttcaactggaagcaggaattcaggactctggaggtcacactccgggatttcagcaagttgaataagcaagatatcagatatctggggaaaatattcagctctgccacaagcctcaggctgcaaataaagaga

SEQ ID NO: 184 - protein, 55 aa, Homo sapiens
TYIPSRAVSLFFNWKQEFRTLEVTLRDFSKLNKQDIRYLGKIFSSATSLRLQIKR

SEQ ID NO: 185 - DNA, 19 bp, Artificial Sequence
  primer
gaaatgtgctcgcaggagg

SEQ ID NO: 186 - DNA, 20 bp, Artificial Sequence
  primer
gatgagcttctgacaggccc

SEQ ID NO: 187 - DNA, 3063 bp, Homo sapiens
  CDS (1)...(2385)
  CDS (2389)...(2928)
tgtgaaatgtgctcgcaggaggcttttcaggcacagaggagccagctggtcgagctgctggtctcagggtccctggaaggcttcgagagtgtcctggactggctgctgtcctgggaggtcctctcctgggaggactacgagggcttccacctcctgggccagcctctctcccacttggccaggcgccttctggacaccgtctggaataagggtacttgggcctgtcagaagctcatcgcggctgcccaagaagcccaggccgacagccagtcccccaagctgcatggctgctgggacccccactcgctccacccagcccgagacctgcagagtcaccggccagccattgtcaggaggctccacagccatgtggagaacatgctggacctggcatgggagcggggtttcgtcagccagtatgaatgtgatgaaatcaggttgccgatcttcacaccgtcccagagggcaagaaggctgcttgatcttgccacggtgaaagcgaatggattggctgccttccttctacaacatgttcaggaattaccagtcccattggccctgcctttggaagctgccacatgcaagaagtatatggccaagctgaggaccacggtgtctgctcagtctcgcttcctcagtacctatgatggagcagagacgctctgcctggaggacatatacacagagaatgtcctggaggtctgggcagatgtgggcatggctggacccccgcagaagagcccagccaccctgggcctggaggagctcttcagcacccctggccacctcaatgacgatgcggacactgtgctggtggtgggtgaggcgggcagtggcaagagcacgctcctgcagcggctgcacttgctgtgggctgcagggcaagacttccaggaatttctctttgtcttcccattcagctgccggcagctgcagtgcatggccaaaccactctctgtgcggactctactctttgagcactgctgttggcctgatgttggtcaagaagacatcttccagttactccttgaccaccctgaccgtgtcctgttaacctttgatggctttgacgagttcaagttcaggttcacggatcgtgaacgccactgctccccgaccgaccccacctctgtccagaccctgctcttcaaccttctgcagggcaacctgctgaagaatgcccgcaaggtggtgaccagccgtccggccgctgtgtcggcgttcctcaggaagtacatccgcaccgagttcaacctcaagggcttctctgaacagggcatcgagctgtacctgaggaagcgccatcatgagcccggggtggcggaccgcctcatccgcctgctccaagagacctcagccctgcacggtttgtgccacctgcctgtcttctcatggatggtgtccaaatgccaccaggaactgttgctgcaggagggggggtccccaaagaccactacagatatgtacctgctgattctgcagcattttctgctgcatgccacccccccagactcagcttcccaaggtctgggacccagtcttcttcggggccgcctccccaccctcctgcacctgggcagactggctctgtggggcctgggcatgtgctgctacgtgttctcagcccagcagctccaggcagcacaggtcagccctgatgacatttctcttggcttcctggtgcgtgccaaaggtgtcgtgccagggagtacggcgcccctggaattccttcacatcactttccagtgcttctttgccgcgttctacctggcactcagtgctgatgtgccaccagctttgctcagacacctcttcaattgtggcaggccaggcaactcaccaatggccaggctcctgcccacgatgtgcatccaggcctcggagggaaaggacagcagcgtggcagctttgctgcagaaggccgagccgcacaaccttcagatcacagcagccttcctggcagggctgttgtcccgggagcactggggcctgctggctgagtgccagacatctgagaaggccctgctccggcgccaggcctgtgcccgctggtgtctggcccgcagcctccgcaagcacttccactccatcccgccagctgcaccgggtgaggccaagagcgtgcatgccatgcccgggttcatctggctcatccggagcctgtacgagatgcaggaggagcggctggctcggaaggctgcacgtggcctgaatgttgggcacctcaagttgacattttgcagtgtgggccccactgagtgtgctgccctggcctttgtgctgcagcacctccggcggcccgtggccctgcagctggactacaactctgtgggtgacattggcgtggagcagctgctgccttgccttggtgtctgcaaggctctgtaattctggggcaacagagtgggtgacgagggggcccaggccctggctgaagccttgggtgatcaccagagcttgaggtggctcagcctggtggggaacaacattggcagtgtgggtgcccaagccttggcactgatgctggcaaagaacgtcatgctagaagaactctgcctggaggagaaccatctccaggatgaaggtgtatgttctctcgcagaaggactgaagaaaaattcaagtttgaaaatcctgaacataaaaattcatgcttcgggattcaacaaactcttggaaagcattttctgcatcctcctggttgtggaagcatttttcctgcagaaagttgtcaagattcttgaagaaatggtagtcagttggctagaggtcaggttgtccaataactgcatcacctacctaggggcagaagccctcctgcaggcccttgaaaggaatgacaccatcctggaagtctggctccgagggaacactttctctctagaggaggttgacaagctcggctgcagggacaccagactcttgctttgaagtctccgggaggatgttcgtctcagtttgtttgtgagcaggctgtgagtttgggccccagaggctgggtgacatgtgttggcagcctcttcaaaatgagccctgtcctgcctaaggctgaacttgttttct

SEQ ID NO: 188 - protein, 795 aa, Homo sapiens
CEMCSQEAFQAQRSQLVELLVSGSLEGFESVLDWLLSWEVLSWEDYEGFHLLGQPLSHLARRLLDTVWNKGTWACQKLIAAAQEAQADSQSPKLHGCWDPHSLHPARDLQSHRPAIVRRLHSHVENMLDLAWERGFVSQYECDEIRLPIFTPSQRARRLLDLATVKANGLAAFLLQHVQELPVPLALPLEAATCKKYMAKLRTTVSAQSRFLSTYDGAETLCLEDIYTENVLEVWADVGMAGPPQKSPATLGLEELFSTPGHLNDDADTVLVVGEAGSGKSTLLQRLHLLWAAGQDFQEFLFVFPFSCRQLQCMAKPLSVRTLLFEHCCWPDVGQEDIFQLLLDHPDRVLLTFDGFDEFKFRFTDRERHCSPTDPTSVQTLLFNLLQGNLLKNARKVVTSRPAAVSAFLRKYIRTEFNLKGFSEQGIELYLRKRHHEPGVADRLIRLLQETSALHGLCHLPVFSWMVSKCHQELLLQEGGSPKTTTDMYLLILQHFLLHATPPDSASQGLGPSLLRGRLPTLLHLGRLALWGLGMCCYVFSAQQLQAAQVSPDDISLGFLVRAKGVVPGSTAPLEFLHITFQCFFAAFYLALSADVPPALLRHLFNCGRPGNSPMARLLPTMCIQASEGKDSSVAALLQKAEPHNLQITAAFLAGLLSREHWGLLAECQTSEKALLRRQACARWCLARSLRKHFHSIPPAAPGEAKSVHAMPGFIWLIRSLYEMQEERLARKAARGLNVGHLKLTFCSVGPTECAALAFVLQHLRRPVALQLDYNSVGDIGVEQLLPCLGVCKAL

SEQ ID NO: 189 - protein, 180 aa, Homo sapiens
FWGNRVGDEGAQALAEALGDHQSLRWLSLVGNNIGSVGAQALALMLAKNVMLEELCLEENHLQDEGVCSLAEGLKKNSSLKILNIKIHASGFNKLLESIFCILLVVEAFFLQKVVKILEEMVVSWLEVRLSNNCITYLGAEALLQALERNDTILEVWLRGNTFSLEEVDKLGCRDTRLLL

SEQ ID NO: 190 - DNA, 721 bp, Mus musculus
  CDS (193)...(612)
cctggggttcctgcacattaccttccgtgcttttttgccgctttctacttggctgtcagtgctgacacatcggtggcctctctcaagcaccttttcagctgtggccggctgggcagctcactgctgggaaggctgctgcccaacctgtgtatccagggctccagagtcaagaagggcagcgaagcagccctgctgcagaaggctgagccacacaacctgcaaatcacagcagccttcctagcaggtctgttgtcccagcagcatcgggacctgttggctgcatgccaggtctccgagagggtactgctccagcgtcaggcacgtgcccgctcgtgtctggcccacagcctccgcgagcacttccattccatcccgcctgccgtgcccggtgagaccaagagcatgcatgctatgccgggcttcatttggctcatccgtagcctgtacgagatgcaggaggagcagttggcccaggaggctgtccgtcgcttggacatcgggcacctgaagttgacattttgcagagtgggccctgcagagtgtgctgcactggcctttgtactgcaacatctccagcggcctgtggccctacagctggattacaactctgtgggagatgttgggagtggaacagctgcgaccgtgcctttggggtctgcacagctctgtagtgagtgtgacaaggtcttgccgattgggcctgtggcaaatgctactgtca

SEQ ID NO: 191 - protein, 140 aa, Mus musculus
LQKAEPHNLQITAAFLAGLLSQQHRDLLAACQVSERVLLQRQARARSCLAHSLREHFHSIPPAVPGETKSMHAMPGFIWLIRSLYEMQEEQLAQEAVRRLDIGHLKLTFCRVGPAECAALAFVLQHLQRPVALQLDYNSV

SEQ ID NO: 192 - DNA, 419 bp, Mus musculus
  CDS (1)...(417)
ctgcagaaggctgagccacacaacctgcagatcacagcagccttcctagcaggtctgttgtcccagcagcatcgggacctgttggctgcatgccagatctccgagagggtgctgctccagcgtcaggcacgtgcccgctcgtgtctggcccacagcctccgcgagcacttccattccatcccgcctgccgtgcccggtgagaccaagagcatgcatgctatgccgggctttatttggctcatccggagcctgtacgagatgcaggaggagcagttggcccaggaggctgtccgtcgcttggacatcgggcacctgaagttgacattttgcagagtgggccctgcagagtgtgctgcgctggcctttgtactgcaacatctccagcggcctgtggccctacagctggattacaactctgt

SEQ ID NO: 193 - protein, 139 aa, Mus musculus
LQKAEPHNLQITAAFLAGLLSQQHRDLLAACQISERVLLQRQARARSCLAHSLREHFHSIPPAVPGETKSMHAMPGFIWLIRSLYEMQEEQLAQEAVRRLDIGHLKLTFCRVGPAECAALAFVLQHLQRPVALQLDYNS

SEQ ID NO: 194 - DNA, 26 bp, Artificial Sequence
  primer
ctgcagaaggctgagccacacaacct

SEQ ID NO: 195 - DNA, 30 bp, Artificial Sequence
  primer
acagagttgtaatccagctgtagggccaca

SEQ ID NO: 196 - DNA, 3042 bp, Homo sapiens
  CDS (1)...(3042)
atgtgctcgcaggaggcttttcaggcacagaggagccagctggtcgagctgctggtctcagggtccctggaaggcttcgagagtgtcctggactggctgctgtcctgggaggtcctctcctgggaggactacgagggcttccacctcctgggccagcctctctcccacttggccaggcgccttctggacaccgtctggaataagggtacttgggcctgtcagaagctcatcgcggctgcccaagaagcccaggccgacagccagtcccccaagctgcatggctgctgggacccccactcgctccacccagcccgagacctgcagagtcaccggccagccattgtcaggaggctccacagccatgtggagaacatgctggacctggcatgggagcggggtttcgtcagccagtatgaatgtgatgaaatcaggttgccgatcttcacaccgtcccagagggcaagaaggctgcttgatcttgccacggtgaaagcgaatggattggctgccttccttctacaacatgttcaggaattaccagtcccattggccctgcctttggaagctgccacatgcaagaagtatatggccaagctgaggaccacggtgtctgctcagtctcgcttcctcagtacctatgatggagcagagacgctctgcctggaggacatatacacagagaatgtcctggaggtctgggcagatgtgggcatggctggacccccgcagaagagcccagccaccctgggcctggaggagctcttcagcacccctggccacctcaatgacgatgcggacactgtgctggtggtgggtgaggcgggcagtggcaagagcacgctcctgcagcggctgcacttgctgtgggctgcagggcaagacttccaggaatttctctttgtcttcccattcagctgccggcagctgcagtgcatggccaaaccactctctgtgcggactctactctttgagcactgctgttggcctgatgttggtcaagaagacatcttccagttactccttgaccaccctgaccgtgtcctgttaacctttgatggctttgacgagttcaagttcaggttcacggatcgtgaacgccactgctccccgaccgaccccacctctgtccagaccctgctcttcaaccttctgcagggcaacctgctgaagaatgcccgcaaggtggtgaccagccgtccggccgctgtgtcggcgttcctcaggaagtacatccgcaccgagttcaacctcaagggcttctctgaacagggcatcgagctgtacctgaggaagcgccatcatgagcccggggtggcggaccgcctcatccgcctgctccaagagacctcagccctgcacggtttgtgccacctgcctgtcttctcatggatggtgtccaaatgccaccaggaactgttgctgcaggagggggggtccccaaagaccactacagatatgtacctgctgattctgcagcattttctgctgcatgccacccccccagactcagcttcccaaggtctgggacccagtcttcttcggggccgcctccccaccctcctgcacctgggcagactggctctgtggggcctgggcatgtgctgctacgtgttctcagcccagcagctccaggcagcacaggtcagccctgatgacatttctcttggcttcctggtgcgtgccaaaggtgtcgtgccagggagtacggcgcccctggaattccttcacatcactttccagtgcttctttgccgcgttctacctggcactcagtgctgatgtgccaccagctttgctcagacacctcttcaattgtggcaggccaggcaactcaccaatggccaggctcctgcccacgatgtgcatccaggcctcggagggaaaggacagcagcgtggcagctttgctgcagaaggccgagccgcacaaccttcagatcacagcagccttcctggcagggctgttgtcccgggagcactggggcctgctggctgagtgccagacatctgagaaggccctgctccggcgccaggcctgtgcccgctggtgtctggcccgcagcctccgcaagcacttccactccatcccgccagctgcaccgggtgaggccaagagcgtgcatgccatgcccgggttcatctggctcatccggagcctgtacgagatgcaggaggagcggctggctcggaaggctgcacgtggcctgaatgttgggcacctcaagttgacattttgcagtgtgggccccactgagtgtgctgccctggcctttgtgctgcagcacctccggcggcccgtggccctgcagctggactacaactctgtgggtgacattggcgtggagcagctgctgccttgccttggtgtctgcaaggctctgtatttgcgcgataacaatatctcagaccgaggcatctgcaagctcattgaatgtgctcttcactgcgagcaattgcagaagttagctctattcaacaacaaattgactgacggctgtgcacactccatggctaagctccttgcatgcaggcagaacttcttggcattgaggctggggaataactacatcactgccgcgggagcccaagtgctggccgaggggctccgaggcaacacctccttgcagttcctgggattctggggcaacagagtgggtgacgagggggcccaggccctggctgaagccttgggtgatcaccagagcttgaggtggctcagcctggtggggaacaacattggcagtgtgggtgcccaagccttggcactgatgctggcaaagaacgtcatgctagaagaactctgcctggaggagaaccatctccaggatgaaggtgtatgttctctcgcagaaggactgaagaaaaattcaagtttgaaaatcctgaagttgtccaataactgcatcacctacctaggggcagaagccctcctgcaggcccttgaaaggaatgacaccatcctggaagtctggctccgagggaacactttctctctagaggaggttgacaagctcggctgcagggacaccagactcttgctttga

SEQ ID NO: 197 - protein, 1013 aa, Homo sapiens
MCSQEAFQAQRSQLVELLVSGSLEGFESVLDWLLSWEVLSWEDYEGFHLLGQPLSHLARRLLDTVWNKGTWACQKLIAAAQEAQADSQSPKLHGCWDPHSLHPARDLQSHRPAIVRRLHSHVENMLDLAWERGFVSQYECDEIRLPIFTPSQRARRLLDLATVKANGLAAFLLQHVQELPVPLALPLEAATCKKYMAKLRTTVSAQSRFLSTYDGAETLCLEDIYTENVLEVWADVGMAGPPQKSPATLGLEELFSTPGHLNDDADTVLVVGEAGSGKSTLLQRLHLLWAAGQDFQEFLFVFPFSCRQLQCMAKPLSVRTLLFEHCCWPDVGQEDIFQLLLDHPDRVLLTFDGFDEFKFRFTDRERHCSPTDPTSVQTLLFNLLQGNLLKNARKVVTSRPAAVSAFLRKYIRTEFNLKGFSEQGIELYLRKRHHEPGVADRLIRLLQETSALHGLCHLPVFSWMVSKCHQELLLQEGGSPKTTTDMYLLILQHFLLHATPPDSASQGLGPSLLRGRLPTLLHLGRLALWGLGMCCYVFSAQQLQAAQVSPDDISLGFLVRAKGVVPGSTAPLEFLHITFQCFFAAFYLALSADVPPALLRHLFNCGRPGNSPMARLLPTMCIQASEGKDSSVAALLQKAEPHNLQITAAFLAGLLSREHWGLLAECQTSEKALLRRQACARWCLARSLRKHFHSIPPAAPGEAKSVHAMPGFIWLIRSLYEMQEERLARKAARGLNVGHLKLTFCSVGPTECAALAFVLQHLRRPVALQLDYNSVGDIGVEQLLPCLGVCKALYLRDNNISDRGICKLIECALHCEQLQKLALFNNKLTDGCAHSMAKLLACRQNFLALRLGNNYITAAGAQVLAEGLRGNTSLQFLGFWGNRVGDEGAQALAEALGDHQSLRWLSLVGNNIGSVGAQALALMLAKNVMLEELCLEENHLQDEGVCSLAEGLKKNSSLKILKLSNNCITYLGAEALLQALERNDTILEVWLRGNTFSLEEVDKLGCRDTRLLL

SEQ ID NO: 198 - DNA, 29 bp, Artificial Sequence
  synthetic oligonucleotide
caagatcaagcagccttcttgccctctgg

SEQ ID NO: 199 - DNA, 30 bp, Artificial Sequence
  synthetic oligonucleotide
catccaggcctcggagggaaaggacagcag

SEQ ID NO: 200 - DNA, 30 bp, Artificial Sequence
  synthetic oligonucleotide
cggaattcatgtgctcgcaggaggcttttc

SEQ ID NO: 201 - DNA, 22 bp, Artificial Sequence
  synthetic oligonucleotide
caagttcagccttaggcaggac

SEQ ID NO: 202 - DNA, 324 bp, Homo sapiens
acgcggggttctccacatggctgtggagcctctagaaccatggccaactcgggttctgctggggctgacttgccctggccttccctgaccaccctgcatctggcttctggagaagtcccgcactgaccttgttctcccccccaggttgtgaaatgtgctcgcaggaggcttttcaggcacagaggagccagctggtcgagctgctggtctcagggtccctggaaggcttcgagagtgtcctggactggctgctgtcctgggaggtcctctcctgggaggactacgagggcttccacctcctgggccagcctctctcccacttgg

SEQ ID NO: 203 - DNA, 81 bp, Homo sapiens
atgggggaagagggtggttcagcctctcacgatgaggaggaaagagcaagtgtcctcctcggacattctccgggttgtgaa

(19) United States
(12) Patent Application Publication
(10) Pub. No.: US 2005/0037378 A1
(43) Pub. Date: Feb. 17, 2005

(54) CARD3X-2 polypeptides, encoding nucleic acids, and methods of use

(76) Inventors: John C. Reed, Rancho Santa Fe, CA (US); Frederick F. Pio, British Columbia (CA); Adam Godzik, San Diego, CA (US); Christian Stehlik, Morgantown, WV (US); Jason S. Damiano, San Diego, CA (US); Sug Hyung Lee, Seoul (KR); Vasco A. Oliveira, San Diego, CA (US); Hideki Hayashi, Nagasaki City (JP); Kryzysztof Pawlowski, Malmo (SE)

Correspondence Address: Cathryn Campbell;McDERMOTT, WILL & EMERY, Suite 700, 4370 La Jolla Village Drive, San Diego, CA, 92122, US

(21) Appl. No.: 10/766,682

(22) Filed: Jan. 27, 2004

Related U.S. Application Data
(63) Continuation-in-part of application No. 09/864,921, filed on May 23, 2001.
(60) Provisional application No. 60/325,756, filed on May 24, 2000.
(60) Provisional application No. 60/367,337, filed on Oct. 10, 2000.
(60) Provisional application No. 60/275,980, filed on Mar. 14, 2001.

(51) Int. Cl. C12Q 1/68; C07H 21/04; C12N 9/64

(52) U.S. Cl. 435/6; 435/69.1; 435/226; 435/320.1; 435/325; 536/23.2

(57) ABSTRACT

The invention provides a polypeptide corresponding to a newly identified splice variant of the CARD3X gene, termed CARD3X-2. Also provided by the invention is an encoding nucleic acid molecule, as well as a CARD3X-2 specific antibody. The invention further provides related screening, diagnostic and therapeutic methods.

This application is a continuation-in-part of U.S. application Ser. No. 09/864,921, filed May 23, 2001, and claims the benefit of three U.S. Provisional Applications: Application Ser. No. 60/325,756, filed May 24, 2000, which was converted from U.S. Ser. No. 09/579,240; and Application No. 60/367,337, filed Oct. 10, 2000, which was converted from U.S. Ser. No. 09/686,347; and Application No. 60/275,980, filed Mar. 14, 2001, each of which is incorporated herein by reference in its entirety.

This invention was made in part with U.S. Government support under NIH Grant No. GM61694 awarded by the National Institutes of Health and DAMD 17-01-1-0166 awarded by the Department of Defense. The U.S. Government has certain rights in this invention.

### BACKGROUND OF THE INVENTION

1. Field of the Invention

This invention relates generally to the fields of molecular biology and molecular medicine and more specifically to the identification of proteins involved in programmed cell death, cytokine processing and receptor signal transduction, and associations of these proteins.

2. Background Information

Programmed cell death is a physiologic process that ensures homeostasis is maintained between cell production and cell turnover in essentially all self-renewing tissues. In many cases, characteristic morphological changes, termed “apoptosis,” occur in a dying cell. Since similar changes occur in different types of dying cells, cell death appears to proceed through a common pathway in different cell types.

In addition to maintaining tissue homeostasis, apoptosis also occurs in response to a variety of external stimuli, including growth factor deprivation, alterations in calcium levels, free-radicals, cytotoxic lymphokines, infection by some viruses, radiation and most chemotherapeutic agents. Thus, apoptosis is an inducible event that likely is subject to similar mechanisms of regulation as occur, for example, in a metabolic pathway. In this regard, dysregulation of apoptosis also can occur and is observed, for example, in some types of cancer cells, which survive for a longer time than corresponding normal cells, and in neurodegenerative diseases where neurons die prematurely. In viral infections, induction of apoptosis can figure prominently in the pathophysiology of the disease process, because immune-based for eradication of viral infections depend on elimination of virus-producing host cells by immune cell attack resulting in apoptosis.

Some of the proteins involved in programmed cell death have been identified and associations among some of these proteins have been described. However, additional apoptosis regulating proteins remain to be found and the mechanisms by which these proteins mediate their activity remains to be elucidated. The identification of the proteins involved in cell death and an understanding of the associations between these proteins can provide a means for manipulating the process of apoptosis in a cell and, therefore, selectively regulating the relative lifespan of a cell or its relative resistance to cell death stimuli.

The principal effectors of apoptosis are a family of intracellular proteases known as Caspases, representing an abbreviation for Cysteine Aspartyl Proteases. Caspases are found as inactive zymogens in essentially all animal cells. During apoptosis, the caspases are activated by proteolytic processing at specific aspartic acid residues, resulting in the production of subunits that assemble into an active protease typically consisting of a heterotetramer containing two large and two small subunits. The phenomenon of apoptosis is produced directly or indirectly by the activation of caspases in cells, resulting in the proteolytic cleavage of specific substrate proteins. Moreover, in many cases, caspases can cleave and activate themselves and each other, creating cascades of protease activation and mechanisms for “auto”-activation. Thus, knowledge about the proteins that interact with and regulate caspases is important for devising strategies for manipulating cell life and death in therapeutically useful ways. In addition, because capsases can also participate in cytokine activation and other processes, knowledge about the proteins that interact with caspases can be important for manipulating immune responses and other biochemical processes in useful ways.

One of the mechanisms for regulating caspase activation involve protein-protein interactions mediated by a family of protein domains known as caspase recruitment domains (CARDs), some of which contain protein domains known as NACHT domains. The identification of proteins that contain CARD and NACHT domains and the identification of their binding partners can therefore form the basis for strategies designed to alter apoptosis, cytokine production, cytokine receptor signaling, and other cellular processes. Thus, a need exists to identify proteins that contain CARD and NACHT domains. The present invention satisfies this need and provides additional advantages as well.

### SUMMARY OF THE INVENTION

The invention provides an isolated nucleic acid molecule encoding a CARD3X-2 polypeptide containing the amino acid sequence set forth as SEQ ID NO:197. In one embodiment, the nucleotide sequence of the nucleic acid molecule contains SEQ ID NO:196. In a further embodiment, the nucleic acid molecule is cDNA. Also provided by the invention is a vector containing the nucleic acid molecule encoding the CARD3X-2 polypeptide, as well as recombinant cells containing the nucleic acid molecule. The invention provides an isolated CARD3X-2 polypeptide that contains the amino acid sequence set forth as SEQ ID NO:197, or a domain of the polypeptide selected from CARD, NACHT and LRR domains. Also provided is a method of producing the CARD3X-2 polypeptide. The method involves expressing the cDNA encoding the CARD3X-2 polypeptide in vitro or in a cell.

The invention provides an isolated anti-CARD3X-2 antibody having specific reactivity with the CARD3X-2 polypeptide. The CARD3X-2 antibody can be used, for example, to distinguish the CARD3X-2 polypeptide from other CARD3X isoform polypeptides. In one embodiment, the antibody is monoclonal; in another embodiment, the antibody is polyclonal. Also provided is cell line producing the CARD3X-2 monoclonal antibody.

The invention provides a method for identifying a nucleic acid molecule encoding a CARD3X-2 polypeptide. The method involves contacting a sample containing nucleic acids with a CARD3X-2 oligonucleotide, wherein the contacting is effected under high stringency hybridization conditions, and identifying a nucleic acid molecule that hybridizes to the oligonucleotide as a nucleic acid molecule encoding a CARD3X-2 polypeptide.

Further provided by the invention is a method for determining the presence of a CARD3X-2 polypeptide in a sample. The method involves contacting a test sample with a CARD3X-2 antibody or recombinant phage, detecting the presence of an antibody:CARD3X-2 complex or recombinant phage:CARD3X-2 complex, thereby detecting the presence of a CARD3X-2 polypeptide in the sample.

The invention also provides a method of identifying a CARD3X-2-binding molecule. The method involves (a) contacting a CARD3X-2 polypeptide with a candidate CARD3X-2-binding molecule, and (b) detecting association of the CARD3X-2 polypeptide with the CARD3X-2-binding molecule. The method can be used, for example, to identify a CARD3X-2-associated polypeptide.

Also provided by the invention is a method of identifying an effective agent that alters association of a NACHT-containing polypeptide with a NACHT-associated polypeptide (NAP). The method involves (a) contacting a NACHT-containing polypeptide selected from SEQ ID NOS: 188, 189 and 97, and the NAP with an agent suspected of being able to alter the association of the NACHT-containing polypeptide and the NAP, under conditions that allow association between the NACHT-containing polypeptide and the NAP; and (b) detecting altered association of the NACHT-containing polypeptide and the NAP, wherein such association identifies an effective agent. In an embodiment, the NAP is selected from CARD3X, CARD3X-2, Nod1, NAC, PAN2, NAIP and cyropyrin.

### BRIEF DESCRIPTION OF THE FIGURES

FIG. 1A shows the genomic organization of the CLAN (CARD 4/5X) gene on chromosome 2 deduced from the BAC 164M19 sequence from the SPG4 candidate region at 2p21-2p22 (Accession No. AL121653) and Homo sapiens chromosome 2 working draft sequence (Accession No. NT_{—}005194.1). FIG. 1B shows mRNA splicing generating CLAN A, B, C and D. FIG. 1C shows the deduced domain structure for the splice forms of CARD4/5X (CLAN A, B, C and D).

FIG. 2 shows an alignment of the protein sequence of the isoforms of CLAN (designated CLAN A, B, C and D; SEQ ID NOS:97, 99, 103 and 101, respectively). Dark boxes indicate identical amino acids, and white boxes indicate conserved amino acids.

FIG. 3 shows the amino acid sequences of the CARD-A, CARD-B and NACHT domains of CARD3X (SEQ ID NOS: 170, 172 and 174, respectively).

FIG. 4 shows an alignment of COP-1 (SEQ ID NO:86) and caspase-1 (SEQ ID NO:87). The amino acids shaded in black are identical.

FIG. 5 shows an alignment of COP-2 (SEQ ID NO:90) and caspase-1 (SEQ ID NO:87), with the consensus sequence (SEQ ID NO:91) shown above the aligned sequences. The amino acids shaded in black are identical.

FIG. 6 shows IL-1β secretion by COS7 cells transfected with the indicated amounts of expression vectors encoding the indicated proteins.

FIG. 7 shows interaction of NACHT-family protein CLAN with itself and NACHT-family proteins CARD3X (Nod2), NAC, and PAN2.

FIG. 8 shows heterotypic NACHT domain interactions between the CLAN NACHT domain and NACHT domains of CARD3X (Nod2), Nod1, NAC, NAIP, cryopyrin, as well as CLAN NACHT domain self association.

FIG. 9 shows gel filtration analysis of the NACHT domain of CLAN, indicating that the CLAN NACHT domain is present in the examined samples in multimer form.

FIG. 10 shows that CLAN inhibits NF-κB activation induced by Nod1 and CARD3X (Nod2).

FIG. 11 shows that CLAN-mediated suppression of CARD3X (Nod2)-induced NF-κB activity is specific and requires only the CLAN NACHT domain.

FIG. 12 shows that CARD family proteins containing NACHT domains bind to pro-caspase 1.

FIG. 13 shows that CLAN inhibits caspase-1 activation induced by CARD3X (Nod2). FIG. 13A shows a decrease in IL-1β secretion induced by CARD3X in the presence of CLAN. FIG. 13B shows a decrease in IL-1β secretion induced by CARD3X[ΔLRR]in the presence of CLAN[ΔLRR]. FIG. 13C shows a decrease in IL-1β secretion induced by CARD3X[ΔLRR] in the presence of either CLAN[ΔLRR] or CLAN NACHT domain.

### DETAILED DESCRIPTION OF THE INVENTION

The present invention provides novel polypeptides involved in cellular functions such as programmed cell death, or apoptosis, and inflammation payways such as NFKB induction and caspase activation. The principal effectors of apoptosis are a family of intracellular cysteine aspartyl proteases, known as caspases. Caspase activity in the cell is regulated by protein-protein interactions. Similarly, protein-protein interactions influence the activity of other proteins involved in apoptosis. Several protein interaction domains have been implicated in interactions among some apoptosis-regulating proteins. Among these is the caspase recruitment domain, or CARD-containing polypeptide which are so named for the ability of the CARD-containing polypeptides to bind caspases. In addition to their ability to bind caspases, numerous CARD-containing polypeptides bind other proteins, particularly, other CARD-containing polypeptides. Further, CARD-containing polypeptides influence a variety of cellular and biochemical processes beyond apoptosis, including cell adhesion, inflammation and cytokine receptor signaling.

In accordance with the present invention, there are provided isolated CARD-containing polypeptides or functional fragments thereof, comprising substantially the same amino acid sequence as set forth in any of SEQ ID NOS: 12, 168, 188, 197, 170, 172, 174, 176, 97, 99, 101, 103, 178, 180, 182, 184, 86 and 90.

The sequence identifiers set forth above correspond to the molecules described herein as set forth in Table 1.

TABLE 1

| | Nucleotide | Polypeptide |
|---|---|---|
| Designation | SEQ ID NO: | SEQ ID NO: |
| CARD2X | 11 | 12 |
| CARD2X CARD Domain | 167 | 168 |
| CARD3X | 187 | 188 and 189 |
| CARD3X CARDA Domain | 169 | 170 |
| CARD3X CARDB Domain | 171 | 172 |
| CARD3X NACHT Domain | 173 | 174 |
| CARD3X ANGIO-R Domain | 175 | 176 |
| CARD3X-2 | 196 | 197 |
| CLAN A | 96 | 97 |
| CLAN B | 98 | 99 |
| CLAN C | 100 | 101 |
| CLAN D | 102 | 103 |
| CLAN CARD | 177 | 178 |
| CLAN NACHT | 179 | 180 |
| CLAN LRR | 181 | 182 |
| CLAN SAM | 183 | 184 |
| COP1 | 85 | 86 |
| COP2 | 89 | 90 |

The terms “CARD-containing protein” or “CARD-containing polypeptide” as used herein refer to a protein or polypeptide containing a CARD domain. As used herein, the term “CARD domain” refers to a Caspase Recruitment Domain. A CARD domain is a well known protein domain of approximately 80 amino acids with characteristic sequence conservation as described, for example, in Hofmann et al., Trends Biochem. Sci. 22:155-156 (1997). CARD domains have been found in some members of the Caspase family of cell death proteases. Caspases-1, 2, 4, 5, 9, and 11 contain CARD domains near their NH2-termini. These CARD domains mediate interactions of the zymogen inactive forms of caspases with other proteins which can either activate or inhibit the activation of these enzymes.

For example, the CARD domain of pro-caspase-9 binds to the CARD domain of a caspase-activating protein called Apaf-1 (Apoptosis Protease Activating Factor-1). Similarly, the CARD domain of pro-caspase-1 permits interactions with another CARD protein known as Cardiac (also referred to as RIP2 and RICK), which results in activation of the caspase-1 protease (Thome et al., Curr. Biol. 16:885-888 (1998)). Additionally, pro-caspase-2 binds to the CARD protein Raidd (also know as Cradd), which permits recruitment of pro-caspase-2 to Tumor Necrosis Factor (TNF) Receptor complexes and which results in activation of the caspase-2 protease (Ahmad et al., Cancer Res. 57:615-619 (1997)). CARD domains can also participate in homotypic interactions with themselves, resulting in self-association of polypeptides that contain these protein-interaction domains and producing dimeric or possibly even oligomeric complexes.

CARD domains can be found in association with other types of functional domains within a single polypeptide, thus providing a mechanism for bringing a functional domain into close proximity or contact with a target protein via CARD:CARD associations involving two CARD-containing polypeptides. For example, the Caenorhabiditis elegans cell death gene ced-4 encodes a protein that contains a CARD domain and a ATP-binding oligomerization domain called a NACHT domain (van der Biezen and Jones, Curr. Biol. 8:R226-R227). The CARD domain of the CED-4 protein interacts with the CARD domain of a pro-caspase called CED-3. The NACHT domain allows CED-4 to self-associate, thereby forming an oligomeric complex which brings associated pro-CED-3 molecules into close proximity to each other. Because most pro-caspases possess at least a small amount of protease activity even in their unprocessed form, the assembly of a complex that brings the proforms of caspase into juxtaposition can result in trans-processing of zymogens, producing the proteolytically processed and active caspase. Thus, CED-4 employs a CARD domain for binding a pro-caspase and a NACHT domain for self-oligomerization, resulting in caspase clustering, proteolytic processing and activation.

In addition to their role in caspase activation, CARD domains have been implicated in other cellular processes. Some CARD-containing polypeptides, for example, induce activation of the transcription factor NF-kB. NF-kB activation is induced by many cytokines and plays an important role in cytokine receptor signal transduction mechanisms (DiDonato et al., Nature 388:548-554 (1997)). Moreover, CARD domains are found in some proteins that inhibit rather than activate caspases, such as the IAP (Inhibitor of Apoptosis Protein) family members, cIAP1 and cIAP2 (Rothe et al., Cell 83:1243-1252 (1995)) and oncogenic mutants of the Bcl-10 protein (Willis et al., Cell 96:35-45 (1999)). Also, though caspase activation resulting from CARD domain interactions is often involved in inducing apoptosis, other caspases are primarily involved in proteolytic processing and activation of inflammatory cytokines (such as pro-IL-1b and pro-IL-18). Thus, CARD-containing polypeptides can also be involved in cytokine receptor signaling and cytokine production, and, therefore, can be involved in regulation of immune and inflammatory responses.

In view of the function of the CARD domain within the invention CARD-containing polypeptides or functional fragments thereof, polypeptides of the invention are contemplated herein for use in methods to alter biochemical processes such as apoptosis, NF-kB induction, cytokine processing, cytokine receptor signaling, caspase-mediated proteolysis, thus having modulating effects on cell life and death (i.e., apoptosis), inflammation, cell adhesion, and other cellular and biochemical processes.

Invention CARD-containing polypeptides or functional fragments thereof (including CARD domains) are also contemplated in methods to identify CARD-binding agents and CARD-associated polypeptides (CAPs) that alter apoptosis, NF-kB induction, cytokine processing, cytokine receptor signaling, caspase-mediated proteolysis, thus having modulating effects on cell life and death (i.e., apoptosis), inflammation, cell adhesion, and other cellular and biochemical processes.

It is also contemplated herein that invention CARD-containing polypeptides can associate with other CARD-containing polypeptides to form invention hetero-oligomers or homo-oligomers, such as heterodimers or homodimers. In particular, the association of the CARD domain of invention polypeptides with other CARD-containing polypeptides, such as Apaf-1, CED-4, caspases-1, 2, 9, 11, cIAPs-1 and 2, CARDIAK, Raidd, Dark, CLAN, other invention CARD-containing polypeptides, and the like, including homo-oligomerization, is sufficiently specific such that the bound complex can form in vivo in a cell or in vitro under suitable conditions. Similarly therefore, an invention CARD-containing polypeptide can associate with another CARD-containing polypeptide by CARD:CARD form invention hetero-oligomers or homo-oligomers, such as heterodimers or homodimers.

In accordance with the present invention, sequences for novel CARD-containing polypeptides have been determined. Thus, the present invention provides novel CARD-containing polypeptides, including the newly identified CARD-containing polypeptides designated CARD2X, CARD3X (Nod2) and alternative splice variant CARD3X-2, CLAN A, CLAN B, CLAN C, CLAN D, COP-1 and COP-2 (set forth in SEQ ID NOS: 12, 188, 197, 97, 99, 101, 103, 86 and 90).

Regarding the newly identified alternatively spliced CARD3X/Nod2 isoform, such alternative splicing can occur in different cell types or at different times during development, giving rise to different cell- or tissue-specific isoforms or developmentally-restricted isoforms. Furthermore, these and other splice variants can encode protein isoforms that have physiological activities that differ in degree or type from related isoforms. An isoform arising from a splice variant can differ, for example, in stability, clearance rate, tissue or cellular localization, tissue expression pattern, temporal pattern of expression, regulation, or response to agonists or antagonists.

In many cases, the presence or level of a specific isoform contributes to, or protects against, a pathological condition. As such, the CARD3X-2 isoform disclosed herein represents a new drug target or diagnostic marker. Because a drug can have differential activity on one isoform compared to another, knowledge of isoforms that represent drug targets can contribute to improved understanding of drug effectiveness, as well as improved drug screening strategies and drug design.

In addition to CARD domains, invention polypeptides can contain one or more additional domains. The locations within the reference sequence of the domains described herein are set forth in Table 2.

A CARD domain containing polypeptide of the invention also can contain a NACHT domain. The term “NACHT-containing protein” or “NACHT-containing polypeptide” as used herein refer to a protein or polypeptide containing a NACHT domain. As used herein, the term “NACHT domain” refers to a combination of an ATP binding domain which can be modeled as a classical Rossman fold followed by a helical domain and containing Walker A and B boxes but lacking several of the features of a NACHT domain. A NACHT domain can associate with another NACHT domain to form invention hetero-oligomers or homo-oligomers, such as heterodimers and homodimers. For example, as disclosed herein the NACHT domain of CLANA can form a complex with a variety of NACHT-containing polypeptides and thereby regulate the activation of caspase-1 via modulating the activation of NF-κB family transcription factors. In particular, the association of the NACHT domain of invention polypeptides with other NACHT-containing polypeptides, such as CLAN, Nod1, CARD3X, CARD3X-2, NAC, PAN2, NAIP, and cyropyrin, and the like, is sufficiently specific such that the bound complex can form in vivo in a cell or in vitro under suitable conditions.

In addition to their role in caspase activation, NACHT domains have been implicated in other cellular processes. Some NACHT-containing polypeptides, for example, induce activation of the transcription factor NF-κB. NF-κB activation is induced by many cytokines and plays an important role in cytokine receptor signal transduction mechanisms (DiDonato et al., Nature 388:548-554 (1997)). Thus, CARD domain-containing polypeptides that contain NACHT domains can also be involved in NFKB induction and cytokine production, and, therefore, can be involved in regulation of immune and inflammatory responses.

In view of the function of the NACHT domain within the invention CARD-containing polypeptides or functional fragments thereof, polypeptides of the invention are contemplated herein for use in methods to alter biochemical processes such as apoptosis, NF-κB induction, cytokine processing, cytokine receptor signaling, caspase-mediated proteolysis, thus having modulating effects on cell life and death (i.e., apoptosis), inflammation, cell adhesion, innate immunity and other cellular and biochemical processes.

Invention NACHT-containing polypeptides or functional fragments thereof that include NACHT domains are also contemplated in methods to identify NACHT-associated polypeptides (NAPs) that alter apoptosis, NF-kB induction, cytokine processing, cytokine receptor signaling, caspase-mediated proteolysis, thus having modulating effects on cell life and death (i.e., apoptosis), inflammation, cell adhesion, and other cellular and biochemical processes.

In accordance with the present invention, sequences for novel CARD-containing polypeptides have been determined. Thus, the present invention provides novel CARD-containing polypeptides, including the newly identified CARD-containing polypeptides designated CARD2X, CARD3X (Nod2) and splice variant CARD3X-2, CLAN A, CLAN B, CLAN C, CLAN D, COP-1 and COP-2 (set forth in SEQ ID NOS: 12, 188, 197, 97, 99, 101, 103, 86 and 90).

TABLE 2

| | Corresponding amino | SEQ ID |
|---|---|---|
| Domain | acids | NO: |
| CARD2X | 4-78 of SEQ ID NO: 12 | 167 (nt) |
| CARD Domain | | 168 (aa) |
| CARD3X | 28-124 of SEQ ID | 169 (nt) |
| CARDA Domain | NO: 107 | 170 (aa) |
| CARD3X | 127-220 of SEQ ID | 171 (nt) |
| CARDB Domain | NO: 107 | 172 (aa) |
| CARD3X | 246-591 of SEQ ID | 173 (nt) |
| NACHT Domain | NO: 107 | 174 (aa) |
| CARD3X | 437-839 of SEQ ID | 175 (nt) |
| ANGIO-R Domain | NO: 107 | 176 (aa) |
| CARD3X | 717-1020 of SEQ ID | 202 (nt) |
| LRR Domain | NO: 107 | 203 (aa) |
| CARD3X-2 | 1-97 of SEQ ID NO: 197 | 204 (nt) |
| CARD A Domain | | 205 (aa) |
| CARD3X-2 | 127-220 of SEQ ID | 206 (nt) |
| CARD B Domain | NO: 197 | 207 (aa) |
| CARD3X-2 | 273-618 of SEQ ID | 208 (nt) |
| NACHT Domain | NO: 197 | 209 (aa) |
| CARD3X-2 | 744-1020 of SEQ ID | 210 (nt) |
| LRR Domain | NO: 197 | 211 (aa) |
| CLAN | 1-87 of SEQ ID NO: 97 | 177 (nt) |
| CARD Domain | | 178 (aa) |
| CLAN | 161-457 of SEQ ID | 179 (nt) |
| NACHT Domain | NO: 97 | 180 (aa) |
| CLAN | 760-965 of SEQ ID | 181 (nt) |
| LRR Domain | NO: 97 | 182 (aa) |
| CLAN | 642-696 of SEQ ID | 183 (nt) |
| SAM Domain | NO: 97 | 184 (aa) |

CARD3X (SEQ ID NO:188) contains at least five distinct domains: two CARD domains, designated CARD-A and CARD-B, a NACHT domain, an LRR domain and an angio-R domain. A second in-frame, open reading frame that begins after a stop codon encodes a domain with several leucine rich repeats (LRR) (SEQ ID NO:189) (see Example). An invention CARD3X polypeptide can thus contain the amino acid sequence designated SEQ ID NO:188 and the amino acid sequence designated SEQ ID NO:189; contain SEQ ID NO:188 but not SEQ ID NO:189; or contain SEQ ID NO:189 but not SEQ ID NO:188. A murine CARD3X polypeptide can contain the amino acid sequence designated SEQ ID NO:193, which is homologous to a portion of the human CARD3X ANGIO-R domain, with or without one or more additional CARD3X domains. Also disclosed herein is a previously unrecognized CARD3X splice variant polypeptide termed CARD3X-2 (SEQ ID NO:197), which is the product of two alternatively spliced exons. CARD3X-2 has an altered 5′ end with respect to the amino acid sequence designated SEQ ID NO:188, with 27 amino acids of SEQ ID NO:188 being absent from CARD3X-2. The 5′ untranslated region of the CARD3X-2 nucleotide acid molecule is referenced herein as SEQ ID NO:202; the nucleotide sequence present in the coding region for CARD3X (SEQ ID NO:187) but is absent in CARD3X-2 (SEQ ID NO:196) is referenced herein as SEQ ID NO:203. As is disclosed herein, CARD3X-2 contains at least two CARD domains, a NACHT domain, an ANGIO-R domain and an LRR domain. The discovery by the inventors of CARD3X-2 SEQ ID NO:197 is described in the Example (16.0).

CLAN exists in four isoforms (see Example), each of which contains a CARD domain. The longest isoform, CLAN-A, also contains a NACHTdomain, a LRR domain and a SAM domain. CLAN represents a new member of the CED-4 related protein family. Numerous CED-4-related proteins have recently been identified. These proteins belong to the CED-4 family of proteins, and include CED-4 (Yuan and Horvitz, Development 116:309-320 (1992)), Apaf-1, (Zou et al., Cell 90:405-413 (1997)), Dark (Rodriguez et al., Nature Cell Biol. 1:272-279 (1999)), and CARD4/Nod1 (Bertin et al., J. Biol. Chem. 274:12955-12958 (1999) and Inohara et al., J. Biol. Chem. 274:14560-14567 (1999)). As used herein, a “CED-4 family” member or “CED-4 protein family” member, also referred to herein as a “NAC” polypeptide, is a polypeptide that comprises a NACHT domain and a CARD domain.

The CED-4 homolog in humans and rodents, referred to as Apaf-1, contains a (i) CARD domain, (ii) NACHT domain, and (iii) multiple copies of a WD-repeat domain. In contrast to CED-4 which can spontaneously oligomerize, the mammalian Apaf-1 protein is an inactive monomer until induced to oligomerize by binding of a co-factor protein, cytochrome c (Li et al., Cell 91:479-489 (1997)). In Apaf-1, the WD repeat domains prevent oligomerization of the Apaf-1 protein, until coming into contact with cytochrome c. Thus, the WD-repeats function as a negative-regulatory domain that maintains Apaf-1 in a latent state until cytochrome c release from damaged mitochondria triggers the assembly of an oligomeric Apaf-1 complex (Saleh, J. Biol. Chem. 274:17941-17945 (1999)). By binding pro-caspase-9 through its CARD domain, Apaf-1 oligomeric complexes are thought to bring the zymogen forms of caspase-9 into close proximity, permitting them to cleave each other and produce the proteolytic processed and active caspase-9 protease (Zou et al., J. Biol. Chem. 274:11549-11556 (1999)).

Another characteristic of the invention CARD-containing polypeptides is that they can associate with pro-caspases, caspases or with caspase-associated proteins, thereby altering caspase proteolytic activity. Caspase proteolytic activity is associated with apoptosis of cells, and additionally with cytokine production. Therefore, an invention CARD-containing polypeptide can alter apoptosis or cytokine production by altering caspase proteolytic activity. As used herein a “caspase” is any member of the cysteine aspartyl proteases. Typically, as caspase can associate with a CARD-containing polypeptide of the invention such as a NAC polypeptide. Similarly, a “pro-caspase” is an inactive or less-active precursor form of a caspase, which is typically converted to the more active caspase form by a proteolytic event, and often a proteolytic event preceded by a protein:protein interaction such as a CARD: CARD interaction, and the like.

As described in the Example, COP-1 interacts with the prodomain of pro-caspase-1 and also with RIP2, a protein previously demonstrated to bind the prodomain of pro-caspase-1. COP-1 competes with RIP2 for binding to pro-caspase-1, thereby inhibiting RIP2-mediated caspase-1 oligomerization. Consequently, COP-1 interferes with the ability of RIP2 to enhance caspase-1-induced secretion of mature IL-1β. Therefore, COP-1 is likely to play a role in controlling IL-1β generation and thereby opposing IL-1β-induced inflammation. IL-1β plays a critical role in septic shock, which currently represents the most common cause of lethality in patients treated in the intensive care setting. Accordingly, COP-1 likely plays a role in IL-1β homeostasis to prevent systemic inflammatory reactions when challenged with gram-negative bacteria or other inflammatory insults.

As also described in the Example, because of their interactions with diverse other CARD proteins, the isoforms of CLAN (A, B, C and D) likely influence apoptosis, cytokine processing, or NF-kB activity. Interactions of CLAN with pro-caspase-1 likely indicates a role for CLAN as a IL-1β regulator. In this regard, different isoforms of CLAN likely have opposing effects on pro-caspase-1 activation. The longest isoform, CLAN-A, for example, can trigger pro-caspase-1 activation by the “induced proximity” mechanism as a result of oligomerization mediated by its NACHT domain. In contrast, the shorter isoforms of CLAN lacking this self-oligomerization can operate as competitive antagonists of pro-caspase-1 activation, analogous to ICEBERG, a CARD-containing protein that competes with CARDIAK (RIP2/RICK) for binding to pro-caspase-1. Interactions of CLAN with NAC also suggest this protein can have an influence on apoptosis mediated by Apaf-1, in as much as NAC binds Apaf-1 and enhances its ability to activate caspase-9 in response to cytochrome c. Finally, CLAN associations with NF-kB regulators such as Bcl-10 and Nod2 strongly suggest that at least some of the CLAN isoforms can influence the activity of this transcription factor.

In addition to the ability to bind caspases, invention CARD-containing polypeptides can contain a protease domain, such as a protease domain found in caspase, and the like. A caspase protease domain hydrolyzes amide bonds, particularly the amide bond of a peptide or polypeptide backbone. Typically, a caspase protease domain contains a P20/P10 domain in the active site region of the caspase protease domain. Thus, a caspase protease domain has proteolytic activity.

CARD-containing polypeptides are also known to induce activation of the transcription factor NF-kB. Thus, an invention CARD-containing polypeptide can also alter transcription by, for example, modulation of NF-kB activity, and the like.

The NACHT domain of invention NAC polypeptides such as CLAN, CARD3X and CARD3X-2 (see Example) associate with other polypeptides, particularly with polypeptides comprising NACHT domains. Thus, a functional NACHT domain associates with NACHT domain-containing polypeptides by way of NACHT:NACHT association. As used herein, the term “associate” or “association” means that CARD-containing polypeptide such as a NAC polypeptide can bind to a polypeptide relatively specifically and, therefore, can form a bound complex. For example, the association of a CARD domain of an invention CARD-containing polypeptide with another CARD-containing polypeptide or the association of a NACHT domain of NAC with another NACHT domain-containing polypeptides is sufficiently specific such that the bound complex can form in vivo in a cell or in vitro under suitable conditions.

As is described in the Example (19.0), NACHT domain of CLAN can associate with several other NACHT domain-containing polypeptides, and this association modulates CLAN NACHT target protein function with respect to NF-κB induction and caspase-1 activation (IL-1β secretion). In particular, shown herein is that CLAN associated with Nod1, CARD3X (Nod2) and NAC(NALP1) through heterotypic NACHT domain interactions when these proteins were co-expressed in HECK293T cells. In addition, NF-κB reporter assays were used to demonstrate that co-expression of either full-length CLAN or the NACHT domain of CLAN significantly inhibited NF-κB activation induced by Nod1 or CARD3X (Nod2) overexpression. Further disclosed herein is that co-expression of CLAN or the NACHT domain of CLAN with Nod1 or CARD3X inhibited the ability of these proteins to generate active IL-1β through their association with pro-caspase-1. In addition, the NACHT domain of CLAN was demonstrated by co-immunoprecipitation experiments to bind all NACHT domains tested, including the NACHT domains from CLAN itself, Nod1, CARD3X, Cryopyrin, NAC, PAN2, and NAIP.

Further, a NACHT domain demonstrates both nucleotide-binding (e.g., ATP-binding) and hydrolysis activities, which is typically required for its ability to associate with NACHT domain-containing polypeptides. Thus, a NACHT domain of the invention NAC comprises one or more nucleotide binding sites. As used herein, a nucleotide binding site is a portion of a polypeptide that specifically binds a nucleotide such as, e.g., ADP, ATP, and the like. Typically, the nucleotide binding site of NACHT will comprise a P-loop, a kinase 2 motif, or a kinase 3a motif of the invention NAC (these motifs are defined, for example, in van der Biezen and Jones, supra). Preferably, the nucleotide binding site of NACHT comprises a P-loop of the invention NAC. The NACHT domain of the an invention CARD-containing polypeptide, therefore, is capable of associating with other NACHT domains in homo- or hetero-oligormerization. Additionally, the NACHT domain is characterized by nucleotide hydrolysis activity, which can influence the ability of a NACHT domain to associate with another NACHT domain.

An invention NAC, therefore, is capable of CARD:CARD association and/or NACHT:NACHT association, resulting in a multifunctional polypeptide capable of one or more specific associations with other polypeptides. An invention NAC can alter cell processes such as apoptosis, cytokine production, inflammatory response, innate immune response and the like. For example, it is contemplated herein that an invention NAC polypeptide can increase the level of apoptosis in a cell. It is also contemplated herein that an invention NAC can decrease the level of apoptosis in a cell. For example, a NAC which does not induce apoptosis may form hetero-oligomers with a NAC which is apoptotic, thus interfering with the apoptosis-inducing activity of NAC. It is further contemplated herein that an invention NAC can modulate inflammatory responses. For example, a NAC can have an ability to modulate the activation of NF-κB family transcription factors as well as in regulating the activation of caspase-1, a protease responsible for generating the secretable, active form of pro-inflammatory cytokine interleukin-1β (IL-1β). Both are NF-κB and IL-1β are involved in regulating inflammation, such as in mounting an effective innate immune response (Li et al., Cell 80:401-11 (1995)).

In another embodiment of the invention, a CARD-containing polypeptide of the invention, such as CLAN (SEQ ID NOS:96, 98, 100 and 102), also contains Leucine-Rich Repeats (LRR) domain. LRR domains are well known in the art and, in one embodiment, the LRR domain of an invention CARD-containing polypeptide has substantially the same sequence as a LRR described in another CARD-containing polypeptide known as Nod1 (Inohara et al., J. Biol. Chem. 274:14560-14567 (1999)). The function of the LRR domain is to mediate specific interactions with other molecules, for example binding to baterical pathogen-derived molecules, such as LPS, peptidoglycan and dimuranyl peptide.

In another embodiment of the invention, there are provided CARD-containing polypeptides that contain a NACHT domain and a CARD domain. NAC polypeptide sequences disclosed herein, for example, CARD4/5X (CLAN), modulate a variety of biochemical processes such as apoptosis. NAC polypeptides can also have other domains that modulate biochemical processes such as an LRR domain or a WD domain.

Those of skill in the art will recognize that numerous residues of the above-described sequences can be substituted with other, chemically, sterically and/or electronically similar residues without substantially altering the biological activity of the resulting CARD-containing polypeptide species. In addition, larger polypeptide sequences comprising substantially the same sequence as amino acids set forth in SEQ ID NOS:12, 168, 188, 170, 172, 174, 176, 97, 99, 101, 103, 178, 180, 182, 184, 86 and 90, therein are contemplated within the scope of the invention.

As employed herein, the term “substantially the same amino acid sequence” refers to amino acid sequences having at least about 70% or 75% identity with respect to the reference amino acid sequence, and retaining comparable functional and biological activity characteristic of the polypeptide defined by the reference amino acid sequence. Preferably, polypeptides having “substantially the same amino acid sequence” will have at least about 80%, 82%, 84%, 86% or 88%, more preferably 90%, 91%, 92%, 93% or 94% amino acid identity with respect to the reference amino acid sequence; with greater than about 95%, 96%, 97%, 98% or 99% amino acid sequence identity being especially preferred. It is recognized, however, that polypeptides or nucleic acids containing less than the described levels of sequence identity arising as splice variants or that are modified by conservative amino acid substitutions, or by substitution of degenerate codons are also encompassed within the scope of the present invention.

In accordance with the invention, specifically included within the definition of substantially the same amino acid sequence is the predominant amino acid sequence of a particular invention CARD-containing polypeptide or domain disclosed herein. The predominant amino acid sequence refers to the most commonly expressed naturally occurring amino acid sequence in a species population. A predominant polypeptide with multiple isoforms will have the most commonly expressed amino acid sequence for each isoform. A predominant CARD-containing polypeptide of the invention refers to an amino acid sequence having sequence identity to an amino acid sequence disclosed herein that is greater than that of any other naturally occurring protein of a particular species (e.g., human).

Given the teachings herein of the location and nucleic acid or amino acid sequences corresponding to the invention CARD-containing polypeptides, one of skill in the art can readily confirm and, if necessary, revise the nucleic acid or amino acid sequences associated with the CARD-containing polypeptides of the invention. For example, the sequences can be confirmed by probing a cDNA library with a nucleic acid probe corresponding to a nucleic acid of the invention using PCR or other known methods. Further, an appropriate bacterial artificial chromosome containing the region of the genome encoding an invention CARD-containing polypeptide can be commercially obtained and probed using PCR, restriction mapping, sequencing, and other known methods.

The term “biologically active” or “functional”, when used herein as a modifier of invention CARD-containing polypeptides, or polypeptide fragments thereof, refers to a polypeptide that exhibits functional characteristics similar to a CARD-containing polypeptide of the invention. Biological activities of a CARD-containing polypeptide include, for example, the ability to bind, preferably in vivo, to a nucleotide, to a CARD-associated polypeptide, to a NACHT-containing polypeptide, or to homo-oligomerize, or to alter protease activation, particularly caspase activation, or to catalyze reactions such as proteolysis or nucleotide hydrolysis, or to alter NF-kB activity, or to alter apoptosis, cytokine processing, cytokine receptor signaling, inflammation, immune response, and other biological activities described herein.

The ability of a CARD-containing polypeptide to bind another polypeptide such as a CARD-associated polypeptide, and the ability of a NACHT-containing polypeptide to bind another NACHT-containing polypeptide, can be assayed, for example, using the methods well known in the art such as yeast two-hybrid assays, co-immunoprecipitation, GST fusion co-purification, and other methods provided in standard technique manuals such as Sambrook, supra, and Ausubel et al., supra. Another biological activity of a CARD-containing polypeptide is the ability to act as an immunogen for the production of polyclonal and monoclonal antibodies that bind specifically to an invention CARD-containing polypeptide. Thus, an invention nucleic acid encoding a CARD-containing polypeptide can encode a polypeptide specifically recognized by an antibody that also specifically recognizes a CARD-containing polypeptide (preferably human) including the amino acid set forth in SEQ ID NOS: 12, 168, 188, 197, 170, 172, 174, 176, 97, 99, 101, 103, 178, 180, 182, 184, 86 and 90. Such immunologic activity may be assayed by any method known to those of skill in the art. For example, a test-polypeptide can be used to produce antibodies, which are then assayed for their ability to bind to an invention polypeptide. If the antibody binds to the test-polypeptide and to the reference polypeptide with substantially the same affinity, then the polypeptide possesses the requisite immunologic biological activity.

As used herein, the term “substantially purified” means a polypeptide that is in a form that is relatively free from contaminating lipids, polypeptides, nucleic acids or other cellular material normally associated with a polypeptide in a cell. A substantially purified CARD-containing polypeptide can be obtained by a variety of methods well-known in the art, e.g., recombinant expression systems described herein, chemical synthesis or purification from native sources. Purification methods can include, for example, precipitation, gel filtration, ion-exchange, reverse-phase and affinity chromatography, and the like. Other well-known methods are described in Deutscher et al., “Guide to Protein Purification” Methods in Enzymology Vol. 182, (Academic Press, (1990)). Alternatively, the isolated polypeptides of the present invention can be obtained using well-known recombinant methods as described, for example, in Sambrook et al., supra, (1989) and Ausubel et al., supra (2000). The methods and conditions for biochemical purification of a polypeptide of the invention can be chosen by those skilled in the art, and purification monitored, for example, by an immunological assay, binding assay, or a functional assay.

In addition to the ability of invention CARD-containing polypeptides, or functional fragments thereof, to interact with other, heterologous proteins (e.g., CARD-containing polypeptides), invention CARD-containing polypeptides have the ability to self-associate to form invention homo-oligomers such as homodimers. This self-association is possible through interactions between CARD domains, and also through interactions between NACHT domains. Further, self-association can take place as a result of interactions between LRR domains.

In accordance with the invention, there are also provided mutations and fragments of CARD-containing polypeptides which have activity different than a predominant naturally occurring CARD-containing polypeptide activity. As used herein, a “mutation” can be any deletion, insertion, or change of one or more amino acids in the predominant naturally occurring protein sequence (e.g., wild-type), and a “fragment” is any truncated form, either carboxy-terminal, amino-terminal, or both, of the predominant naturally occurring protein. Preferably, the different activity of the mutation or fragment is a result of the mutant polypeptide or fragment maintaining some but not all of the activities of the respective predominant naturally occurring CARD-containing polypeptide.

For example, a functional fragment of an invention polypeptide can contain or consist of one or more of the following: a CARD domain, a NACHT domain, a LRR domain, a SAM domain, or an angio-R domain. In a specific example, a fragment of a CARD-containing polypeptide such as CLAN can contain a CARD domain and LRR domain, but lack a functional NACHT domain. Such a fragment will maintain a portion of the predominant naturally occurring CLAN activity (e.g., CARD domain functionality), but not all such activities (e.g., lacking an active NACHT domain). The resultant fragment will therefore have an activity different than the predominant naturally occurring CLAN activity. In another example, the CLAN polypeptide might have only the NACHT domain, allowing it to interact with other NACHT domain proteins in forming homo-oligomers or hetero-oligomers. In one embodiment, the activity of the fragment will be “dominant-negative.” A dominant-negative activity will allow the fragment to reduce or inactivate the activity of one or more isoforms of a predominant naturally occurring CARD-containing polypeptide. Another functional fragment can include an angio-R domain (see Example), or any of the domains disclosed herein (see, for example, Table 2).

Isoforms of the CARD-containing polypeptides are also provided which arise from alternative mRNA splicing and may alter or modify the interactions of the CARD-containing polypeptide with other polypeptides. For example, four isoforms of CLAN and four isoforms of CARD3X are disclosed herein. Additional isoforms of the CARD-containing polypeptides designated SEQ ID NOS: 12, 188, 197, 97, 99, 101, 103, 86 and 90, are contemplated herein and therefore, are encompassed within the scope of the invention CARD-containing polypeptides.

Methods to identify polypeptides containing a functional fragment of a CARD-containing polypeptide of the invention are well known in the art and are disclosed herein. For example, genomic or cDNA libraries, including universal cDNA libraries can be probed according to methods disclosed herein or other methods known in the art. Full-length polypeptide encoding nucleic acids such as full-length cDNAs can be obtained by a variety of methods well-known in the art. For example, 5′ and 3′ RACE, methodology is well known in the art and described in Ausubel et al., supra, and the like.

In another embodiment of the invention, chimeric polypeptides are provided comprising a CARD-containing polypeptide, or a functional fragment thereof, fused with another protein or functional fragment thereof. Functional fragments of a CARD-containing polypeptide include, for example, NACHT, CARD, LRR, and ANGIO-R domains or other fragments that retain a biological activity of an invention CARD-containing polypeptide. Polypeptides with which the CARD-containing polypeptide or functional fragment thereof are fused will include, for example, glutathione-S-transferase, an antibody, or other proteins or functional fragments thereof which facilitate recovery of the chimera. Further, polypeptides with which a CARD-containing polypeptide or functional fragment thereof are fused will include, for example, luciferase, green fluorescent protein, an antibody, or other proteins or functional fragments thereof which facilitate identification of the chimera. Still further polypeptides with which a CARD-containing polypeptide or functional fragment thereof are fused will include, for example, the LexA DNA binding domain, ricin, a-sarcin, an antibody or fragment thereof, or other polypeptides which have therapeutic properties or other biological activity.

Further invention chimeric polypeptides contemplated herein are chimeric polypeptides wherein a functional fragment of a CARD-containing polypeptide is fused with a catalytic domain or a protein interaction domain from a heterologous polypeptide. For example, the NACHT domain of CLAN, as disclosed herein, can be replaced by the NACHT domain of other CARD polypeptides, such as CARD3X, and the like. Another example of such a chimera is a polypeptide wherein the CARD domain of CLAN is replaced by the CARD domain from CARD2X or CARD3X, and the like. In a further example, a NACHT domain can be fused with a caspase catalytic P20 domain to form a novel chimera with caspase activity. One of skill in the art will appreciate that a large number of chimeric polypeptides are readily available by combining domains of two or more CARD-containing polypeptides of the invention. Further, chimeric polypeptides can contain a functional fragment of a CARD-containing polypeptide of the invention fused with a domain of a protein known in the art, such as CED-4, Apaf-1, caspase-1, and the like.

As used herein, the term “polypeptide” when used in reference to a CARD-containing polypeptide or fragment is intended to refer to a peptide or polypeptide of two or more amino acids. The term “polypeptide analog” includes any polypeptide having an amino acid residue sequence substantially the same as a sequence specifically described herein in which one or more residues have been conservatively substituted with a functionally similar residue and which displays the ability to functionally mimic a CARD-containing polypeptide as described herein. A “modification” of an invention polypeptide also encompasses conservative substitutions of an invention polypeptide amino acid sequence. Conservative substitutions of encoded amino acids include, for example, amino acids that belong within the following groups: (1) non-polar amino acids (Gly, Ala, Val, Leu, and Ile); (2) polar neutral amino acids (Cys, Met, Ser, Thr, Asn, and Gln); (3) polar acidic amino acids (Asp and Glu); (4) polar basic amino acids (Lys, Arg and His); and (5) aromatic amino acids (Phe, Trp, Tyr, and His). Other groupings of amino acids can be found, for example in Taylor, J. Theor. Biol. 119:205-218 (1986), which is incorporated herein by reference. Other minor modifications are included within invention polypeptides so long as the polypeptide retains some or all of its function as described herein.

The amino acid length of functional fragments or polypeptide analogs of the present invention can range from about 5 amino acids up to the full-length protein sequence of an invention CARD-containing polypeptide. In certain embodiments, the amino acid lengths include, for example, at least about 10 amino acids, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 125, at least about 150, at least about 175, at least about 200, at least about 250 or more amino acids in length up to the full-length CARD-containing polypeptide sequence. The functional fragments can be contiguous amino acid sequences of an invention polypeptide, including contiguous amino acid sequences of SEQ ID NOS: 12, 188, 197, 97, 99, 101, 103, 86 and 90. A peptide of at least about 10 amino acids can be used, for example, as an immungen to raise antibodies specific for an invention CARD-containing polypeptide.

A modification of a polypeptide can also include derivatives, analogues and functional mimetics thereof, provided that such polypeptide displays a CARD-containing polypeptide biological activity. For example, derivatives can include chemical modifications of the polypeptide such as alkylation, acylation, carbamylation, iodination, or any modification that derivatizes the polypeptide. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups can be derivatized to form salts, methyl and ethyl esters or other types of esters or hydrazides. Free hydroxyl groups can be derivatized to form O-acyl or O-alkyl derivatives. The imidazole nitrogen of histidine can be derivatized to form N-im-benzylhistidine. Also included as derivatives or analogues are those peptides which contain one or more naturally occurring amino acid derivatives of the twenty standard amino acids, for example, 4-hydroxyproline, 5-hydroxylysine, 3-methylhistidine, homoserine, ornithine or carboxyglutamate, and can include amino acids that are not linked by peptide bonds. Polypeptides of the present invention also include any polypeptide having one or more additions and/or deletions of residues, relative to the sequence of a polypeptide whose sequence is shown herein, so long as CARD-containing polypeptide activity is maintained.

A modification of an invention polypeptide includes functional mimetics thereof. Mimetics encompass chemicals containing chemical moieties that mimic the function of the polypeptide. For example, if a polypeptide contains two charged chemical moieties having functional activity, a mimetic places two charged chemical moieties in a spatial orientation and constrained structure so that the charged chemical function is maintained in three-dimensional space. Thus, a mimetic, which orients functional groups that provide a function of a CARD-containing polypeptide, are included within the meaning of a CARD-containing polypeptide derivative. All of these modifications are included within the term “polypeptide” so long as the invention polypeptide or functional fragment retains its function. Exemplary mimetics are peptidomimetics, peptoids, or other peptide-like polymers such as poly(b-amino acids), and also non-polymeric compounds upon which functional groups that mimic a peptide are positioned.

Another embodiment of the invention provides a CARD-containing polypeptide, or a functional fragment thereof, fused with a moiety to form a conjugate. As used herein, a “moiety” can be a physical, chemical or biological entity which contributes functionality to a CARD-containing polypeptide or a functional fragment thereof. Functionalities contributed by a moiety include therapeutic or other biological activity, or the ability to facilitate identification or recovery of a CARD-containing polypeptide. Therefore, a moiety will include molecules known in the art to be useful for detection of the conjugate by, for example, by fluorescence, magnetic imaging, detection of radioactive emission. A moiety may also be useful for recovery of the conjugate, for example a His tag or other known tags used for protein isolation and/or purification, or a physical substance such as a bead. A moiety can be a therapeutic compound, for example, a cytotoxic drug which can be useful to effect a biological change in cells to which the conjugate localizes.

An example of the means for preparing the invention polypeptide(s) is to express nucleic acids encoding a CARD-containing polypeptide in a suitable host cell, such as a bacterial cell, a yeast cell, an amphibian cell such as an oocyte, or a mammalian cell, using methods well known in the art, and recovering the expressed polypeptide, again using well-known purification methods. Invention polypeptides can be isolated directly from cells that have been transformed with expression vectors as known in the art. Recombinantly expressed polypeptides of the invention can also be expressed as fusion proteins with appropriate affinity tags, such as glutathione S transferase (GST) or poly His, and affinity purified. The invention polypeptide, biologically functional fragments, and functional equivalents thereof can also be produced by in vitro transcription/translation methods known in the art, such as using reticulocyte lysates, as used for example, in the TNT system (Promega). The invention polypeptide, biologically functional fragments, and functional equivalents thereof can also be produced by chemical synthesis. For example, synthetic polypeptides can be produced using Applied Biosystems, Inc. Model 430A or 431A automatic peptide synthesizer (Foster City, Calif.) employing the chemistry provided by the manufacturer.

In an embodiment, the invention provides an isolated nucleic acid molecule encoding a CARD3X-2 polypeptide comprising the amino acid sequence set forth as SEQ ID NO:197. The nucleic acid molecule can contain, for example, the nucleotide sequence set forth as SEQ ID NO:196.

The nucleic acid molecules described herein are useful for producing invention polypeptides, when such nucleic acids are incorporated into a variety of protein expression systems known to those of skill in the art. In addition, such nucleic acid molecules or fragments thereof can be labeled with a readily detectable substituent and used as hybridization probes for assaying for the presence and/or amount of an invention CARD-encoding gene or mRNA transcript in a given sample. The nucleic acid molecules described herein, and fragments thereof, are also useful as primers and/or templates in a PCR reaction for amplifying genes encoding invention polypeptides described herein.

The term “nucleic acid” (also referred to as polynucleotides) encompasses ribonucleic acid (RNA) or deoxyribonucleic acid (DNA), probes, oligonucleotides, and primers and can be single stranded or double stranded. DNA can be either complementary DNA (cDNA) or genomic DNA, e.g. a CARD-encoding gene, and can represent the sense strand, the anti-sense strand, or both. Examples of nucleic acids are RNA, cDNA, or isolated genomic DNA encoding a CARD-containing polypeptide. One means of isolating a CARD-encoding nucleic acid is to probe a mammalian genomic or cDNA library with a natural or artificially designed DNA probe using methods well known in the art. DNA probes derived from the CARD-encoding gene are particularly useful for this purpose. DNA and cDNA molecules that encode CARD-containing polypeptides can be used to obtain complementary genomic DNA, cDNA or RNA from mammalian (e.g., human, mouse, rat, rabbit, pig, and the like), or other animal sources, or to isolate related cDNA or genomic clones by screening cDNA or genomic libraries, using methods described in more detail below. Such nucleic acids include, but are not limited to, nucleic acids comprising substantially the same nucleotide sequence as set forth in SEQ ID NOS: 11, 167, 187, 196, 169, 171, 173, 175, 96, 98, 100, 102, 177, 179, 181, 183, 85 and 89. In general, a genomic sequence of the invention includes regulatory regions such as promoters, enhancers, and introns that are outside of the exons encoding a CARD-containing polypeptide but does not include proximal genes that do not encode a CARD-containing polypeptide.

Thus a CARD-encoding nucleic acid as used herein refers to a nucleic acid encoding a CARD-containing polypeptide of the invention, or a functional fragment thereof.

Use of the terms “isolated” and/or “purified” and/or “substantially purified” in the present specification and claims as a modifier of DNA, RNA, polypeptides or proteins means that the DNA, RNA, polypeptides or proteins so designated have been produced in such form by the hand of man, and thus are separated from their native in vivo cellular environment, and are substantially free of any other species of nucleic acid or protein. As a result of this human intervention, the recombinant DNAs, RNAs, polypeptides and proteins of the invention are useful in ways described herein that the DNAs, RNAs, polypeptides or proteins as they naturally occur are not.

Invention nucleic acids encoding CARD-containing polypeptides and invention CARD-containing polypeptides can be obtained from any species of organism, such as prokaryotes, eukaryotes, plants, fungi, vertebrates, invertebrates, and the like. A particular species can be mammalian, As used herein, “mammalian” refers to a subset of species from which an invention CARD-encoding nucleic acid is derived, e.g., human, rat, mouse, rabbit, monkey, baboon, bovine, porcine, ovine, canine, feline, and the like. A preferred CARD-encoding nucleic acid herein, is human CARD-encoding nucleic acid.

In one embodiment of the present invention, cDNAs encoding the invention CARD-containing polypeptides disclosed herein comprise substantially the same nucleotide sequence as the coding region set forth in any of SEQ ID NOS: 11, 167, 187, 196, 169, 171, 173, 175, 96, 98, 100, 102, 177, 179, 181, 183, 85 and 89.

As employed herein, the term “substantially the same nucleotide sequence” refers to a nucleic acid molecule (DNA or RNA) having sufficient identity to the reference polynucleotide, such that it will hybridize to the reference nucleotide under moderately or highly stringent hybridization conditions. In another embodiment, a nucleic acid molecule having “substantially the same nucleotide sequence” as the reference nucleotide sequence has at least 60%, or at least 65% identity with respect to the reference nucleotide sequence, such as at least 70%, 72%, 74%, 76%, 78%, 80%, 82%, 84%, 86%, 88%, 90%, 91%, 92%, 93%, 94%, 95%, 96%, 97%, 98% or 99% identity to the reference nucleotide sequence.

In accordance with the invention, specifically included within the definition of substantially the same nucleotide sequence is the predominant nucleotide sequence of a particular invention CARD-containing polypeptide described herein. The predominant nucleotide sequence refers to the most commonly present naturally occurring nucleotide sequence in a species population. A predominant CARD-encoding nucleic acid of the invention refers to a nucleotide sequence having sequence identity to a nucleotide sequence disclosed herein that is greater than that of any other naturally occurring nucleotide sequence of a particular species (e.g., human).

In one embodiment, a nucleic acid molecule that has substantially the same nucleotide sequence as a reference sequence is a modification of the reference sequence. As used herein, a “modification” of a nucleic acid can include one or several nucleotide additions, deletions, or substitutions with respect to a reference sequence. A modification of a nucleic acid can include substitutions that do not change the encoded amino acid sequence due to the degeneracy of the genetic code. Such modifications can correspond to variations that are made deliberately, or which occur as mutations during nucleic acid replication.

Exemplary modifications of the recited nucleotide sequences include sequences that correspond to homologs of other species, including mammalian species such as mouse, primates, including monkey and baboon, rat, rabbit, bovine, porcine, ovine, canine, feline, or other animal species. The corresponding nucleotide sequences of non-human species can be determined by methods known in the art, such as by PCR or by screening genomic, cDNA or expression libraries.

Another exemplary modification of the invention CARD-encoding nucleic acid or CARD-containing polypeptide can correspond to splice variant forms of the CARD-encoding nucleotide sequence. Additionally, a modification of a nucleotide sequence can include one or more non-native nucleotides, having, for example, modifications to the base, the sugar, or the phosphate portion, or having a modified phosphodiester linkage. Such modifications can be advantageous in increasing the stability of the nucleic acid molecule.

Furthermore, a modification of a nucleotide sequence can include, for example, a detectable moiety, such as a radiolabel, a fluorochrome, a ferromagnetic substance, a luminescent tag or a detectable binding agent such as biotin. Such modifications can be advantageous in applications where detection of a CARD-encoding nucleic acid molecule is desired.

In another embodiment, a nucleic acid molecule that has substantially the same nucleotide sequence as a reference sequence is a functionally equivalent nucleic acid, which indicates that it is phenotypically similar to the reference nucleic acid. As used herein, the phrase “functionally equivalent nucleic acids” encompasses nucleic acids characterized by slight and non-consequential sequence variations that will function in substantially the same manner to produce the same polypeptide product(s) as the nucleic acids disclosed herein. In particular, functionally equivalent nucleic acids encode polypeptides that are the same as those encoded by the nucleic acids disclosed herein or that have conservative amino acid variations, as described above. These variations include those recognized by skilled artisans as those that do not substantially alter the tertiary structure of the protein.

Further provided are nucleic acids encoding CARD-containing polypeptides that, by virtue of the degeneracy of the genetic code, do not necessarily hybridize to the invention nucleic acids under specified hybridization conditions. Preferred nucleic acids encoding the invention CARD-containing polypeptides are comprised of nucleotides that encode substantially the same amino acid sequence as set forth in SEQ ID NOS:12, 168, 188, 197, 170, 172, 174, 176, 97, 99, 101, 103, 178, 180, 182, 184, 86 and 90.

Hybridization refers to the binding of complementary strands of nucleic acid (i.e., sense:antisense strands or probe:target-DNA) to each other through hydrogen bonds, similar to the bonds that naturally occur in chromosomal DNA. Stringency levels used to hybridize a given probe with target-DNA can be readily varied by those of skill in the art.

The phrase “stringent hybridization” is used herein to refer to conditions under which polynucleic acid hybrids are stable. As known to those of skill in the art, the stability of hybrids is reflected in the melting temperature (Tm) of the hybrids. In general, the stability of a hybrid is a function of sodium ion concentration and temperature. Typically, the hybridization reaction is performed under conditions of lower stringency, followed by washes of varying, but higher, stringency. Reference to hybridization stringency relates to such washing conditions.

As used herein, the phrase “moderately stringent hybridization” refers to conditions that permit target-nucleic acid to bind a complementary nucleic acid. The hybridized nucleic acids will generally have at least about 60% identity, at least about 75% identity, such as at least about 85% identity; or at least about 90% identity. Moderately stringent conditions are conditions equivalent to hybridization in 50% formamide, 5× Denhart's solution, 5×SSPE, 0.2% SDS at 42° C., followed by washing in 0.2×SSPE, 0.2% SDS, at 42° C.

The phrase “high stringency hybridization” refers to conditions that permit hybridization of only those nucleic acid sequences that form stable hybrids in 0.018M NaCl at 65° C., for example, if a hybrid is not stable in 0.018M NaCl at 65° C., it will not be stable under high stringency conditions, as contemplated herein. High stringency conditions can be provided, for example, by hybridization in 50% formamide, 5× Denhart's solution, 5×SSPE, 0.2% SDS at 42° C., followed by washing in 0.1×SSPE, and 0.1% SDS at 65° C.

The phrase “low stringency hybridization” refers to conditions equivalent to hybridization in 10% formamide, 5× Denhart's solution, 6×SSPE, 0.2% SDS at 22° C., followed by washing in 1×SSPE, 0.2% SDS, at 37° C. Denhart's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M (EDTA). Other suitable moderate stringency and high stringency hybridization buffers and conditions are well known to those of skill in the art and are described, for example, in Sambrook et al., supra (1989); and Ausubel et al., supra, 2000). Nucleic acids encoding polypeptides hybridize under moderately stringent or high stringency conditions to substantially the entire sequence, or substantial portions, for example, typically at least 15-30 nucleotides of the nucleic acid sequence set forth in SEQ ID NOS:11, 167, 187, 169, 171, 173, 175, 96, 98, 100, 102, 177, 179, 181, 183, 85, 89 and 212.

As used herein, the term “degenerate” refers to codons that differ in at least one nucleotide from a reference nucleic acid, e.g., SEQ ID NOS:11, 167, 187, 196, 169, 171, 173, 175, 96, 98, 100, 102, 177, 179, 181, 183, 85 and 89, but encode the same amino acids as the reference nucleic acid. For example, codons specified by the triplets “UCU”, “UCC”, “UCA”, and “UCG” are degenerate with respect to each other since all four of these codons encode the amino acid serine.

The invention also provides a modification of a nucleotide sequence that hybridizes to a CARD-encoding nucleic acid molecule, for example, a nucleic acid molecule referenced as any of SEQ ID NOS:11, 167, 187, 196, 169, 171, 173, 175, 96, 98, 100, 102, 177, 179, 181, 183, 85 and 89 under moderately stringent conditions. Modifications of nucleotide sequences, where the modification has at least 60% identity to a CARD-encoding nucleotide sequence, are also provided. The invention also provides modification of a CARD-encoding nucleotide sequence having at least 65% identity, at least 70% identity, at least 72% identity, at least 74% identity, at least 76% identity, at least 78% identity, at least 80% identity, at least 82% identity, at least 84% identity, at least 86% identity, at least 88% identity, at least 90% identity, at least 91% identity, at least 92% identity, at least 93% identity, at least 94% identity, at least 95% identity, at least 96% identity, at least 97% identity, at least 98% identity or at least 99% identity.

Identity of any two nucleic acid or amino acid sequences can be determined by those skilled in the art based, for example, on a BLAST 2.0 computer alignment, using default parameters. BLAST 2.0 searching is known in the art and is publicly available, for example, at http://www.ncbi.nlm.nih.gov/BLAST/, as described by Tatiana et al., FEMS Microbiol Lett. 174:247-250 (1999); Altschul et al., Nucleic Acids Res., 25:3389-3402 (1997).

One means of isolating a nucleic acid encoding a CARD-containing polypeptide is to probe a cDNA library or genomic library with a natural or artificially designed nucleic acid probe using methods well known in the art. Nucleic acid probes derived from a CARD-encoding gene are particularly useful for this purpose. DNA and cDNA molecules that encode CARD-containing polypeptides can be used to obtain complementary genomic DNA, cDNA or RNA from mammals, for example, human, mouse, rat, rabbit, pig, and the like, or other animal sources, or to isolate related cDNA or genomic clones by the screening of cDNA or genomic libraries, by methods well known in the art (see, for example, the Examples set forth hereinafter; and Sambrook et al., supra, 1989; Ausubel et al., supra, 2000).

Another useful method for producing a CARD-encoding nucleic acid molecule of the invention involves amplification of the nucleic acid molecule using PCR and invention oligonucleotides and, optionally, purification of the resulting product by gel electrophoresis. Either PCR or RT-PCR can be used to produce a CARD-encoding nucleic acid molecule having any desired nucleotide boundaries as described in the Examples. Desired modifications to the nucleic acid sequence can also be introduced by choosing an appropriate oligonucleotide primer with one or more additions, deletions or substitutions. Such nucleic acid molecules can be amplified exponentially starting from as little as a single gene or mRNA copy, from any cell, tissue or species of interest.

The invention additionally provides a nucleic acid that hybridizes under high stringency conditions to the CARD coding portion of any of SEQ ID NOS:11, 187, 96, 98, 100, 102, 85 and 89, such as to any of SEQ ID NOS: 168, 170, 172 and 178. The invention also provides a nucleic acid having a nucleotide sequence substantially the same as set that forth in any of SEQ ID 11, 167, 187, 196, 169, 171, 173, 175, 96, 98, 100, 102, 177, 179, 181, 183, 85 and 89.

The invention also provides a method for identifying nucleic acids encoding a mammalian CARD-containing polypeptide by contacting a sample containing nucleic acids with one or more invention nucleic acid molecules or oligonucleotides, wherein the contacting is effected under high stringency hybridization conditions, and identifying a nucleic acid that hybridizes to the oligonucleotide. The invention additionally provides a method of detecting a CARD-encoding nucleic acid molecule in a sample by contacting the sample with two or more invention oligonucleotides, amplifying a nucleic acid molecule, and detecting the amplification. The amplification can be performed, for example, using PCR. The invention further provides oligonucleotides that function as single stranded nucleic acid primers for amplification of a CARD-encoding nucleic acid, wherein the primers comprise a nucleic acid sequence derived from the nucleic acid sequences set forth as SEQ ID NOS:11, 187, 96, 98, 100, 102, 85, and 89.

In accordance with a further embodiment of the present invention, optionally labeled CARD-encoding cDNAs, or fragments thereof, can be employed to probe library(ies) such as cDNA, genomic, BAC, and the like for predominant nucleic acid sequences or additional nucleic acid sequences encoding novel CARD-containing polypeptides. Construction and screening of suitable mammalian cDNA libraries, including human cDNA libraries, is well-known in the art, as demonstrated, for example, in Ausubel et al., supra. Screening of such a cDNA library is initially carried out under low-stringency conditions, which comprise a temperature of less than about 42° C., a formamide concentration of less than about 50%, and a moderate to low salt concentration.

Probe-based screening conditions can comprise a temperature of about 37° C., a formamide concentration of about 20%, and a salt concentration of about 5×standard saline citrate (SSC; 20×SSC contains 3M sodium chloride, 0.3M sodium citrate, pH 7.0). Such conditions will allow the identification of sequences which have a substantial degree of similarity with the probe sequence, without requiring perfect homology. The phrase “substantial similarity” refers to sequences which share at least 50% homology. Hybridization conditions are selected which allow the identification of sequences having at least 70% homology with the probe, while discriminating against sequences which have a lower degree of homology with the probe. As a result, nucleic acids having substantially the same nucleotide sequence as any of SEQ ID NOS:11, 167, 187, 196, 169, 171, 173, 175, 96, 98, 100, 102, 177, 179, 181, 183, 85 and 89 are obtained.

As used herein, a nucleic acid “probe” is single-stranded nucleic acid, or analog thereof, that has a sequence of nucleotides that includes at least 15, at least 20, at least 50, at least 100, at least 200, at least 300, at least 400, or at least 500 contiguous bases that are substantially the same as, or the complement of, any contiguous bases set forth in any of SEQ ID NOS:11, 187, 96, 98, 100, 102, 85, 89 and 212. In addition, the entire cDNA encoding region of an invention CARD-containing polypeptide, or an entire sequence substantially the same as SEQ ID NOS:11, 187, 196, 96, 98, 100, 102, 85 and 89 can be used as a probe. Probes can be labeled by methods well-known in the art, as described hereinafter, and used in various diagnostic kits.

The invention additionally provides an oligonucleotide comprising between 15 and 300 contiguous nucleotides of any of SEQ ID NOS:11, 187, 212, 96, 98, 100, 102, 85 and 89 or the anti-sense strand thereof. As used herein, the term “oligonucleotide” refers to a nucleic acid molecule that includes at least 15 contiguous nucleotides from a reference nucleotide sequence, can include at least 16, 17, 18, 19, 20 or at least 25 contiguous nucleotides, and often includes at least 30, 40, 50, 60, 70, 80, 90, 100, 125, 150, 175, 200, 225, 250, 275, 300, 325, up to 350 contiguous nucleotides from the reference nucleotide sequence. The reference nucleotide sequence can be the sense strand or the anti-sense strand.

The oligonucleotides of the invention that contain at least 15 contiguous nucleotides of a reference CARD-encoding nucleotide sequence are able to hybridize to CARD-encoding nucleotide sequences under moderately stringent hybridization conditions and thus can be advantageously used, for example, as probes to detect CARD-encoding DNA or RNA in a sample, and to detect splice variants thereof; as sequencing or PCR primers; as antisense reagents to block transcription of CARD-encoding RNA in cells; or in other applications known to those skilled in the art in which hybridization to a CARD-encoding nucleic acid molecule is desirable.

In accordance with another embodiment of the invention, a method is provided for identifying nucleic acids encoding a CARD-containing polypeptide. The method comprises contacting a sample containing nucleic acids with an invention probe or an invention oligonucleotide, wherein the contacting is effected under high stringency hybridization conditions, and identifying nucleic acids which hybridize thereto. Methods for identification of nucleic acids encoding a CARD-containing polypeptide are disclosed herein and exemplified in the Examples.

Also provided in accordance with present invention is a method for identifying a CARD-encoding nucleotide sequence comprising the steps of using a CARD-encoding nucleotide sequence selected from SEQ ID NOS:11, 167, 187, 196, 169, 171, 173, 175, 96, 98, 100, 102, 177, 179, 181, 183, 85 and 89 to identify a candidate CARD-encoding nucleotide sequence and verifying the candidate CARD-encoding nucleotide sequence by aligning the candidate sequence with known CARD-encoding nucleotide sequences, where a conserved CARD domain sequence or a predicted three dimensional polypeptide structure similar to a known CARD domain three dimensional structure confirms the candidate sequence as a CARD-encoding sequence. Methods for identifying CARD-encoding sequences are provided herein (See Examples).

An oligonucleotide of the invention can be used in a variety of formats, including solid and solution phases. For example, in a method of the invention for identifying a nucleic acid molecule encoding a CARD3X-2 polypeptide, the selected CARD3X-2 oligonucleotide can be in solution phase with the sample to be tested in solid phase; the selected oligonucleotide can be in solid phase with the sample to be tested in solution phase; and both the oligonucleotide and sample to be tested can be in solution phase. Examples of solid phases include materials such as matrices, beads, sample plates, microspheres, and the like, that are substantially insoluble in the selected solution phase; examples of solution phases include any non-solid medium suitable for conducting a nucleic acid hybridization reaction, such as a liquid and gel. Exemplary conditions for nucleic acid hybridization are described herein above and are well known to those skilled in the art.

It is understood that a CARD-encoding nucleic acid molecule of the invention, as used herein, specifically excludes previously known nucleic acid molecules consisting of nucleotide sequences having identity with the CARD-encoding nucleotide sequence (SEQ ID NOS:11, 167, 187, 196, 169, 171, 173, 175, 96, 98, 100, 102, 177, 179, 181, 183, 85 and 89), such as Expressed Sequence Tags (ESTs), Sequence Tagged Sites (STSs) and genomic fragments, deposited in public databases such as the nr, dbest, dbsts, gss and htgs databases, which are available for searching at http://www.ncbi.nlm.nih.gov/blast/.

In particular, an invention CARD-encoding nucleic acid molecule excludes the exact, specific and complete nucleic acid molecule sequence corresponding to any of the nucleotide sequences having the Genbank (gb), EMBL (emb) or DDBJ (dbj) accession numbers described below. Accession numbers specifically excluded include GI:6165147 (Phase-1), AC007728 (Phase-1), NT-002476 (Phase-1), AC010968 (Phase-1), AP001153, AC022468 (Phase-1), GI:6253000 (Phase-1), AC0097959 (Phase-1), GI:6497652 (Phase-1) (contig:23086:40635), GI:6497652 (Phase-1) (contig:41136:57024), AC023068 (Phase-1), W58453, AA257158, AA046000, AW085161, AI189838, AA418021, AA046105, W58488, AA418193, AA257066, AI217611, AW295205, AI023795, AL389934, AA070591, AA070591, AC027011, AP002787, AQ889169, AV719179, AI263294, AV656315, AW337918, BF207840, AW418826, BK903662, AI023795, H25984, AL121653, and NT_{—}005194.1. The human contig referenced as GenBank accession Nos. AC007608 and AC007728 are also specifically excluded from a CARD encoding nucleic acid molecule. The genomic contigs referenced as GenBank accession numbers GI 5001450, GI 8575872 and GI 9795562 are also specifically excluded from invention nucleic acid molecules. Since one of skill in the art will realize that the above-recited excluded sequences may be revised at a later date, the skilled artisan will recognize that the above-recited sequences are excluded as they stand on the priority date of this application.

The isolated nucleic acid molecules of the invention can be used in a variety of diagnostic and therapeutic applications. For example, the isolated nucleic acid molecules of the invention can be used as probes, as described above; as templates for the recombinant expression of CARD-containing polypeptides; or in screening assays such as two-hybrid assays to identify cellular molecules that bind CARD-containing polypeptides.

The invention thus provides methods for detecting a CARD-encoding nucleic acid in a sample. The methods of detecting a CARD-encoding nucleic acid in a sample can be either qualitative or quantitative, as desired. For example, the presence, abundance, integrity or structure of a CARD-encoding nucleic acid can be determined, as desired, depending on the assay format and the probe used for hybridization or primer pair chosen for application.

Useful assays for detecting a CARD-containing nucleic acid based on specific hybridization with an isolated invention oligonucleotide are well known in the art and include, for example, in situ hybridization, which can be used to detect altered chromosomal location of the nucleic acid molecule, altered gene copy number, and RNA abundance, depending on the assay format used. Other hybridization assays include, for example, Northern blots and RNase protection assays, which can be used to determine the abundance and integrity of different RNA splice variants, and Southern blots, which can be used to determine the copy number and integrity of DNA. A hybridization probe can be labeled with any suitable detectable moiety, such as a radioisotope, fluorochrome, chemiluminescent marker, biotin, or other detectable moiety known in the art that is detectable by analytical methods.

As used herein, the terms “label” and “indicating means” in their various grammatical forms refer to single atoms and molecules that are either directly or indirectly involved in the production of a detectable signal. Any label or indicating means can be linked to invention nucleic acid probes, expressed proteins, polypeptide fragments, or antibody molecules. These atoms or molecules can be used alone or in conjunction with additional reagents. Such labels are themselves well-known in clinical diagnostic chemistry.

Useful assays for detecting a CARD-encoding nucleic acid in a sample based on amplifying a CARD-encoding nucleic acid with two or more invention oligonucleotides are also well known in the art, and include, for example, qualitative or quantitative polymerase chain reaction (PCR); reverse-transcription PCR (RT-PCR); single strand conformational polymorphism (SSCP) analysis, which can readily identify a single point mutation in DNA based on differences in the secondary structure of single-strand DNA that produce an altered electrophoretic mobility upon non-denaturing gel electrophoresis; and coupled PCR, transcription and translation assays, such as a protein truncation test, in which a mutation in DNA is determined by an altered protein product on an electrophoresis gel. Additionally, the amplified CARD-encoding nucleic acid can be sequenced to detect mutations and mutational hot-spots, and specific assays for large-scale screening of samples to identify such mutations can be developed.

Also provided are antisense-nucleic acids having a sequence capable of binding specifically with full-length or any portion of an mRNA that encodes CARD-containing polypeptides so as to prevent translation of the mRNA. The antisense-nucleic acid can have a sequence capable of binding specifically with any portion of the sequence of the cDNA encoding CARD-containing polypeptides. As used herein, the phrase “binding specifically” encompasses the ability of a nucleic acid sequence to recognize a complementary nucleic acid sequence and to form double-helical segments therewith via the formation of hydrogen bonds between the complementary base pairs. An example of an antisense-nucleic acid is an antisense-nucleic acid comprising chemical analogs of nucleotides.

The present invention provides means to alter levels of expression of CARD-containing polypeptides by recombinantly expressing CARD-containing anti-sense nucleic acids or employing synthetic anti-sense nucleic acid compositions (hereinafter SANC) that inhibit translation of mRNA encoding these polypeptides. Synthetic oligonucleotides, or other antisense-nucleic acid chemical structures designed to recognize and selectively bind to mRNA are constructed to be complementary to full-length or portions of a CARD-encoding strand, including nucleotide sequences substantially the same as SEQ ID NOS:11, 187, 196, 96, 98, 100, 102, 85 and 89.

The SANC is designed to be stable in the blood stream for administration to a subject by injection, or in laboratory cell culture conditions. The SANC is designed to be capable of passing through the cell membrane in order to enter the cytoplasm of the cell by virtue of physical and chemical properties of the SANC, which render it capable of passing through cell membranes, for example, by designing small, hydrophobic SANC chemical structures, or by virtue of specific transport systems in the cell which recognize and transport the SANC into the cell. In addition, the SANC can be designed for administration only to certain selected cell populations by targeting the SANC to be recognized by specific cellular uptake mechanisms which bind and take up the SANC only within select cell populations. In a particular embodiment the SANC is an antisense oligonucleotide.

For example, the SANC may be designed to bind to a receptor found only in a certain cell type, as discussed above. The SANC is also designed to recognize and selectively bind to target mRNA sequence, which can correspond to a sequence contained within the sequences shown in SEQ ID NOS:11, 187, 196, 96, 98, 100, 102, 85 and 89. The SANC is designed to inactivate target mRNA sequence by either binding thereto and inducing degradation of the mRNA by, for example, RNase I digestion, or inhibiting translation of mRNA target sequence by interfering with the binding of translation-regulating factors or ribosomes, or inclusion of other chemical structures, such as ribozyme sequences or reactive chemical groups which either degrade or chemically modify the target mRNA. SANCs have been shown to be capable of such properties when directed against mRNA targets (see Cohen et al., TIPS, 10:435 (1989) and Weintraub, Sci. American, January (1990), pp.40).

The invention further provides a method of altering the level of a biochemical process modulated by a CARD-containing polypeptide by introducing an antisense nucleotide sequence into the cell, wherein the antisense nucleotide sequence specifically hybridizes to a CARD-encoding nucleic acid molecule, wherein the hybridization reduces or inhibits the expression of the CARD-containing polypeptide in the cell. The use of anti-sense nucleic acids, including recombinant anti-sense nucleic acids or SANCs, can be advantageously used to inhibit cell death.

Compositions comprising an amount of the antisense-nucleic acid of the invention, effective to reduce expression of CARD-containing polypeptides by entering a cell and binding specifically to CARD-encoding mRNA so as to prevent translation and an acceptable hydrophobic carrier capable of passing through a cell membrane are also provided herein. Suitable hydrophobic carriers are described, for example, in U.S. Pat. Nos. 5,334,761; 4,889,953; 4,897,355, and the like. The acceptable hydrophobic carrier capable of passing through cell membranes may also comprise a structure which binds to a receptor specific for a selected cell type and is thereby taken up by cells of the selected cell type. For example, the structure can be part of a protein known to bind to a cell-type specific receptor such as a tumor.

Antisense-nucleic acid compositions are useful to inhibit translation of mRNA encoding invention polypeptides. Synthetic oligonucleotides, or other antisense chemical structures are designed to bind to CARD-encoding mRNA and inhibit translation of mRNA and are useful as compositions to inhibit expression of CARD-encoding genes or CARD-associated polypeptide genes in a tissue sample or in a subject.

The invention provides additional mRNA targeting molecules capable of selectively binding to an mRNA corresponding to a nucleic acid molecule of the invention, such as a nucleic acid molecule comprising the nucleotide sequence referenced as SEQ ID NO:196, or a portion thereof. Such an mRNA targeting molecule can be, for example, an si RNA or a ribozyme.

An si RNA is a double-stranded RNA molecule for use in RNA interference methods. RNA interference (RNAi) is a process of sequence-specific gene silencing by post-transcriptional RNA degradation, which is initiated by double-stranded RNA (dsRNA) homologous in sequence to the silenced gene. A suitable si RNA for RNAi contains sense and antisense strands of about 21 contiguous nucleotides corresponding to the gene to be targeted that form 19 RNA base pairs, leaving overhangs of two nucleotides at each 3′ end (Elbashir et al., Nature 411:494-498 (2001); Bass, Nature 411:428-429 (2001); Zamore, Nat. Struct. Biol. 8:746-750 (2001)). Si RNAs of about 25-30 nucleotides have also been used successfully for RNAi (Karabinos et al., Proc. Natl. Acad. Sci. 98:7863-7868 (2001). Si RNA can be synthesized in vitro and introduced into a cell by methods known in the art.

The use of ribozymes is described herein below in relation to SANCs. Methods of preparing and using hairpin and hammerhead ribozymes for the selective inhibition of gene expression are known in the art and are described, for example, in Poeschla et al., Curr. Opin. Oncol. 6:601-606 (1994).

The invention also provides vectors containing the CARD-encoding nucleic acids of the invention. Suitable expression vectors are well-known in the art and include vectors capable of expressing nucleic acid operatively linked to a regulatory sequence or element such as a promoter region or enhancer region that is capable of regulating expression of such nucleic acid. Appropriate expression vectors include those that are replicable in eukaryotic cells and/or prokaryotic cells and those that remain episomal or those which integrate into the host cell genome.

Promoters or enhancers, depending upon the nature of the regulation, can be constitutive or regulated. The regulatory sequences or regulatory elements are operatively linked to a nucleic acid of the invention such that the physical and functional relationship between the nucleic acid and the regulatory sequence allows transcription of the nucleic acid.

Suitable vectors for expression in prokaryotic or eukaryotic cells are well known to those skilled in the art (see, for example, Ausubel et al., supra, 2000). Vectors useful for expression in eukaryotic cells can include, for example, regulatory elements including the SV40 early promoter, the cytomegalovirus (CMV) promoter, the mouse mammary tumor virus (MMTV) steroid-inducible promoter, Moloney murine leukemia virus (MMLV) promoter, and the like. The vectors of the invention are useful for subcloning and amplifying a CARD-encoding nucleic acid molecule and for recombinantly expressing a CARD-containing polypeptide. A vector of the invention can include, for example, viral vectors such as a bacteriophage, a baculovirus or a retrovirus; cosmids or plasmids; and, particularly for cloning large nucleic acid molecules, bacterial artificial chromosome vectors (BACs) and yeast artificial chromosome vectors (YACs). Such vectors are commercially available, and their uses are well known in the art. One skilled in the art will know or can readily determine an appropriate promoter for expression in a particular host cell.

The invention additionally provides recombinant cells containing CARD-encoding nucleic acids of the invention. The recombinant cells are generated by introducing into a host cell a vector containing a CARD-encoding nucleic acid molecule. The recombinant cells are transducted, transfected or otherwise genetically modified. Exemplary host cells that can be used to express recombinant CARD molecules include mammalian primary cells; established mammalian cell lines, such as COS, CHO, HeLa, NIH3T3, HEK 293 and PC12 cells; amphibian cells, such as Xenopus embryos and oocytes and other vertebrate cells. Exemplary host cells also include insect cells such as Drosophila, yeast cells such as Saccharomyces cerevisiae, Saccharomyces pombe, or Pichia pastoris, and prokaryotic cells such as Escherichia coli. Additional host cells can be obtained, for example, from ATCC (Manassas, Va.).

In one embodiment, CARD-encoding nucleic acids can be delivered into mammalian cells, either in vivo or in vitro using suitable vectors well-known in the art. Suitable vectors for delivering a CARD-containing polypeptide, or a functional fragment thereof to a mammalian cell, include viral vectors such as retroviral vectors, adenovirus, adeno-associated virus, lentivirus, herpesvirus, as well as non-viral vectors such as plasmid vectors. Such vectors are useful for providing therapeutic amounts of a CARD-containing polypeptide (see, for example, U.S. Pat. No. 5,399,346, issued Mar. 21, 1995). Delivery of CARD polypeptides or nucleic acids therapeutically can be particularly useful when targeted to a tumor cell, thereby inducing apoptosis in tumor cells. In addition, where it is desirable to limit or reduce the in vivo expression of a CARD-containing polypeptide, the introduction of the antisense strand of the invention nucleic acid is contemplated.

Viral based systems provide the advantage of being able to introduce relatively high levels of the heterologous nucleic acid into a variety of cells. Suitable viral vectors for introducing an invention CARD-encoding nucleic acid into mammalian cells are well known in the art. These viral vectors include, for example, Herpes simplex virus vectors (Geller et al., Science, 241:1667-1669 (1988)); vaccinia virus vectors (Piccini et al., Meth. Enzymology, 153:545-563 (1987)); cytomegalovirus vectors (Mocarski et al., in Viral Vectors, Y. Gluzman and S. H. Hughes, Eds., Cold Spring Harbor Laboratory, Cold Spring Harbor, N.Y., 1988, pp. 78-84)); Moloney murine leukemia virus vectors (Danos et al., Proc. Natl. Acad. Sci. USA, 85:6460-6464 (1988); Blaese et al., Science, 270:475-479 (1995); Onodera et al., J. Virol., 72:1769-1774 (1998)); adenovirus vectors (Berkner, Biotechniques, 6:616-626 (1988); Cotten et al., Proc. Natl. Acad. Sci. USA, 89:6094-6098 (1992); Graham et al., Meth. Mol. Biol., 7:109-127 (1991); Li et al., Human Gene Therapy, 4:403-409 (1993); Zabner et al., Nature Genetics, 6:75-83 (1994)); adeno-associated virus vectors (Goldman et al., Human Gene Therapy, 10:2261-2268 (1997); Greelish et al., Nature Med., 5:439-443 (1999); Wang et al., Proc. Natl. Acad. Sci. USA, 96:3906-3910 (1999); Snyder et al., Nature Med., 5:64-70 (1999); Herzog et al., Nature Med., 5:56-63 (1999)); retrovirus vectors (Donahue et al., Nature Med., 4:181-186 (1998); Shackleford et al., Proc. Natl. Acad. Sci. USA, 85:9655-9659 (1988); U.S. Pat. Nos. 4,405,712, 4,650,764 and 5,252,479, and WIPO publications WO 92/07573, WO 90/06997, WO 89/05345, WO 92/05266 and WO 92/14829; and lentivirus vectors (Kafri et al., Nature Genetics, 17:314-317 (1997)).

For example, in one embodiment of the present invention, adenovirus-transferrin/polylysine-DNA (TfAdpl-DNA) vector complexes (Wagner et al., Proc. Natl. Acad. Sci., USA, 89:6099-6103 (1992); Curiel et al., Hum. Gene Ther., 3:147-154 (1992); Gao et al., Hum. Gene Ther., 4:14-24 (1993)) are employed to transduce mammalian cells with heterologous CARD-encoding nucleic acid. Any of the plasmid expression vectors described herein may be employed in a TfAdpl-DNA complex.

Vectors useful for therapeutic administration of a CARD-encoding nucleic acid can contain a regulatory element that provides tissue specific or inducible expression of an operatively linked nucleic acid. One skilled in the art can readily determine an appropriate tissue-specific promoter or enhancer that allows expression of a CARD polypeptide or nucleic acid in a desired tissue. Any of a variety of inducible promoters or enhancers can also be included in the vector for regulatable expression of a CARD polypeptide or nucleic acid. Such inducible systems, include, for example, tetracycline inducible system (Gossen & Bizard, Proc. Natl. Acad. Sci. USA, 89:5547-5551 (1992); Gossen et al., Science, 268:1766-1769 (1995); Clontech, Palo Alto, Calif.); metalothionein promoter induced by heavy metals; insect steroid hormone responsive to ecdysone or related steroids such as muristerone (No et al., Proc. Natl. Acad. Sci. USA, 93:3346-3351 (1996); Yao et al., Nature, 366:476-479 (1993); Invitrogen, Carlsbad, Calif.); mouse mammory tumor virus (MMTV) induced by steroids such as glucocortocoid and estrogen (Lee et al., Nature, 294:228-232 (1981); and heat shock promoters inducible by temperature changes.

An inducible system particularly useful for therapeutic administration utilizes an inducible promoter that can be regulated to deliver a level of therapeutic product in response to a given level of drug administered to an individual and to have little or no expression of the therapeutic product in the absence of the drug. One such system utilizes a Gal4 fusion that is inducible by an antiprogestin such as mifepristone in a modified adenovirus vector (Burien et al., Proc. Natl. Acad. Sci. USA, 96:355-360 (1999). Another such inducible system utilizes the drug rapamycin to induce reconstitution of a transcriptional activator containing rapamycin binding domains of FKBP12 and FRAP in an adeno-associated virus vector (Ye et al., Science, 283:88-91 (1999)). It is understood that any combination of an inducible system can be combined in any suitable vector, including those disclosed herein. Such a regulatable inducible system is advantageous because the level of expression of the therapeutic product can be controlled by the amount of drug administered to the individual or, if desired, expression of the therapeutic product can be terminated by stopping administration of the drug.

The invention also provides a method for expression of a CARD-containing polypeptide by culturing cells containing a CARD-encoding nucleic acid under conditions suitable for expression of a CARD-containing polypeptide. Thus, there is provided a method for the recombinant production of a CARD-containing polypeptide of the invention by expressing the CARD-encoding nucleic acid sequences in suitable host cells. Recombinant DNA expression systems that are suitable to produce a CARD-containing polypeptide described herein are well-known in the art (see, for example, Ausubel et al., supra, 2000). For example, the above-described nucleotide sequences can be incorporated into vectors for further manipulation. As used herein, vector refers to a recombinant DNA or RNA plasmid or virus containing discrete elements that are used to introduce heterologous DNA into cells for either expression or replication thereof.

The invention additionally provides an isolated anti-CARD antibody having specific reactivity with a invention CARD-containing polypeptide. The anti-CARD antibody can be a monoclonal antibody or a polyclonal antibody. The invention further provides cell lines producing monoclonal antibodies having specific reactivity with an invention CARD-containing protien.

The invention thus provides antibodies that specifically bind a CARD-containing polypeptide. As used herein, the term “antibody” is used in its broadest sense to include polyclonal and monoclonal antibodies, as well as antigen binding fragments of such antibodies. With regard to an anti-CARD antibody of the invention, the term “antigen” means a native or synthesized CARD-containing polypeptide or fragment thereof. An anti-CARD antibody, or antigen binding fragment of such an antibody, is characterized by having specific binding activity for a CARD polypeptide or a peptide portion thereof of at least about 1×10⁵ M⁻¹. Thus, Fab, F(ab′)₂, Fd and Fv fragments of an anti-CARD antibody, which retain specific binding activity for a CARD-containing polypeptide, are included within the definition of an antibody. Specific binding activity of a CARD-containing polypeptide can be readily determined by one skilled in the art, for example, by comparing the binding activity of an anti-CARD antibody to a CARD-containing polypeptide versus a reference polypeptide that is not a CARD-containing polypeptide. Methods of preparing polyclonal or monoclonal antibodies are well known to those skilled in the art (see, for example, Harlow and Lane, Antibodies: A Laboratory Manual, Cold Spring Harbor Laboratory Press (1988)).

In addition, the term “antibody” as used herein includes naturally occurring antibodies as well as non-naturally occurring antibodies, including, for example, single chain antibodies, chimeric, bifunctional and humanized antibodies, as well as antigen-binding fragments thereof. Such non-naturally occurring antibodies can be constructed using solid phase peptide synthesis, can be produced recombinantly or can be obtained, for example, by screening combinatorial libraries consisting of variable heavy chains and variable light chains as described by Huse et al., Science 246:1275-1281 (1989)). These and other methods of making, for example, chimeric, humanized, CDR-grafted, single chain, and bifunctional antibodies are well known to those skilled in the art (Winter and Harris, Immunol. Today 14:243-246 (1993); Ward et al., Nature 341:544-546 (1989); Harlow and Lane, supra, 1988); Hilyard et al., Protein Engineering: A practical approach (IRL Press 1992); Borrabeck, Antibody Engineering, 2d ed. (Oxford University Press 1995)).

Anti-CARD antibodies can be raised using a CARD immunogen such as an isolated CARD-containing polypeptide having substantially the same amino acid sequence as SEQ ID NO:197, or fragment thereof, which can be prepared from natural sources or produced recombinantly, or a peptide portion of the CARD-containing polypeptide. Such peptide portions of a CARD-containing polypeptide are functional antigenic fragments if the antigenic peptides can be used to generate a CARD-specific antibody. A non-immunogenic or weakly immunogenic CARD-containing polypeptide or portion thereof can be made immunogenic by coupling the hapten to a carrier molecule such as bovine serum albumin (BSA) or keyhole limpet hemocyanin (KLH). Various other carrier molecules and methods for coupling a hapten to a carrier molecule are well known in the art (see, for example, Harlow and Lane, supra, 1988). An immunogenic CARD-containing polypeptide fragment can also be generated by expressing the peptide as a fusion protein, for example, to glutathione S transferase (GST), polyHis or the like. Methods for expressing peptide fusions are well known to those skilled in the art (Ausubel et al., supra, (2000)).

The invention further provides a method for detecting the presence of a human CARD-containing polypeptide in a sample by contacting a sample with a CARD-specific antibody, and detecting the presence of specific binding of the antibody to the sample, thereby detecting the presence of a human CARD-containing polypeptide in the sample. CARD-specific antibodies can be used in diagnostic methods and systems to detect the level of CARD-containing polypeptide present in a sample. As used herein, the term “sample” is intended to mean any biological fluid, cell, tissue, organ or portion thereof, that includes or potentially includes CARD nucleic acids or polypeptides. The term includes samples present in an individual as well as samples obtained or derived from the individual. For example, a sample can be a histologic section of a specimen obtained by biopsy, or cells that are placed in or adapted to tissue culture. A sample further can be a subcellular fraction or extract, or a crude or substantially pure nucleic acid or polypeptide preparation.

CARD-specific antibodies can also be used for the immunoaffinity or affinity chromatography purification of an invention CARD-containing polypeptide. In addition, methods are contemplated herein for detecting the presence of an invention CARD-containing polypeptide in a cell, comprising contacting the cell with an antibody that specifically binds to CARD-containing polypeptides under conditions permitting binding of the antibody to the CARD-containing polypeptides, detecting the presence of the antibody bound to the CARD-containing polypeptide, and thereby detecting the presence of invention polypeptides in a cell. With respect to the detection of such polypeptides, the antibodies can be used for in vitro diagnostic or in vivo imaging methods.

Immunological procedures useful for in vitro detection of target CARD-containing polypeptides in a sample include immunoassays that employ a detectable antibody. Such immunoassays include, for example, immunohistochemistry, immunofluorescence, ELISA assays, radioimmunoassay, FACS analysis, immunoprecipitation, immunoblot analysis, Pandex microfluorimetric assay, agglutination assays, flow cytometry and serum diagnostic assays, which are well known in the art (Harlow and Lane, supra, 1988; Harlow and Lane, Using Antibodies: A Laboratory Manual, Cold Spring Harbor Press (1999)).

An antibody can be made detectable by various means well known in the art. For example, a detectable marker can be directly attached to the antibody or indirectly attached using, for example, a secondary agent that recognizes the CARD specific antibody. Useful markers include, for example, radionucleotides, enzymes, binding proteins such as biotin, fluorogens, chromogens and chemiluminescent labels.

An antibody can also be detectable by, for example, a fluorescent labeling agent that chemically binds to antibodies or antigens without denaturation to form a fluorochrome (dye) that is a useful immunofluorescent tracer. A description of immunofluorescent analytic techniques is found in DeLuca, “Immunofluorescence Analysis”, in Antibody As a Tool, Marchalonis et al., eds., John Wiley & Sons, Ltd., pp. 189-231 (1982), which is incorporated herein by reference.

In one embodiment, the indicating group is an enzyme, such as horseradish peroxidase (HRP), glucose oxidase, and the like. In another embodiment, radioactive elements are employed labeling agents. The linking of a label to a substrate, i.e., labeling of nucleic acid probes, antibodies, polypeptides, and proteins, is well known in the art. For instance, an invention antibody can be labeled by metabolic incorporation of radiolabeled amino acids provided in the culture medium. See, for example, Galfre et al., Meth. Enzymol., 73:3-46 (1981). Conventional means of protein conjugation or coupling by activated functional groups are particularly applicable. See, for example, Aurameas et al., Scand. J. Immunol., Vol. 8, Suppl. 7:7-23 (1978), Rodwell et al., Biotech., 3:889-894 (1984), and U.S. Pat. No. 4,493,795.

In addition to detecting the presence of a CARD-containing polypeptide, invention anti-CARD antibodies are contemplated for use herein to alter the activity of the CARD-containing polypeptide in living animals, in humans, or in biological tissues or fluids isolated therefrom. The term “alter” refers to the ability of a compound such as a CARD-containing polypeptide, a CARD-encoding nucleic acid, an agent or other compound to increase or decrease biological activity which is modulated by the compound, by functioning as an agonist or antagonist of the compound. Accordingly, compositions comprising a carrier and an amount of an antibody having specificity for CARD-containing polypeptides effective to block naturally occurring ligands or other CARD-associated polypeptides from binding to invention CARD-containing polypeptides are contemplated herein. For example, a monoclonal antibody directed to an epitope of an invention CARD-containing polypeptide, including an amino acid sequence substantially the same as SEQ ID NO:197 can be useful for this purpose. An antibody that has specificity for CARD3X-2 can be used, for example, to distinguish a CARD3X-2 polypeptide from another CARD3X polypeptide isoform. Although the CARD3X-2 polypeptide contains a portion of the CARD3X amino acid sequence, the CARD3X-2 polypeptide can have a conformation that differs from CARD3X. An antibody that can distinguish between the conformation of CARD3X-2 and another CARD3X isoform is useful for identifying the presence of a CARD3X-2 polypeptide.

Also provided by the invention are recombinant phage that have specificity for a CARD or NACHT polypeptide of the invention. Invention CARD or NACHT specific recombinant phage are contemplated for use herein to detect the presence of a CARD or NACHT polypeptide in a sample, as well as to alter the activity of the CARD-containing polypeptide. A recombinant phage used in a method of the invention can be rendered detectable using a variety of detectable markers. Useful markers include, for example, radionucleotides, enzymes, binding proteins such as biotin, fluorogens, chromogens and chemiluminescent labels. Methods of screening to identify recombinant phage are well known to those skilled in the art and are described, for example, in Roovers et al., Br J Cancer. 78(11):1407-16 (1998). In addition, commercial kits are available for preparing recombinant phage antibodies (for example, “Recombinant Phage Antibody System” Amersham Biosciences, Piscataway, N.J.).

The present invention further provides transgenic non-human mammals that are capable of expressing exogenous nucleic acids encoding CARD-containing polypeptides. As employed herein, the phrase “exogenous nucleic acid” refers to nucleic acid sequence which is not native to the host, or which is present in the host in other than its native environment, for example, as part of a genetically engineered DNA construct. In addition to naturally occurring CARD-containing polypeptide levels, a CARD-containing polypeptide of the invention can either be overexpressed or underexpressed in transgenic mammals, for example, underexpressed in a knock-out animal.

Also provided are transgenic non-human mammals capable of expressing CARD-encoding nucleic acids so mutated as to be incapable of normal activity. Therefore, the transgenic non-human mammals do not express native CARD-containing polypeptide or have reduced expression of native CARD-containing polypeptide. The present invention also provides transgenic non-human mammals having a genome comprising antisense nucleic acids complementary to CARD-encoding nucleic acids, placed so as to be transcribed into antisense mRNA complementary to CARD-encoding mRNA, which hybridizes to the mRNA and, thereby, reduces the translation thereof. The nucleic acid can additionally comprise an inducible promoter and/or tissue specific regulatory elements, so that expression can be induced, or restricted to specific cell types.

Animal model systems useful for elucidating the physiological and behavioral roles of CARD-containing polypeptides are also provided, and are produced by creating transgenic animals in which the expression of the CARD-containing polypeptide is altered using a variety of techniques. Examples of such techniques include the insertion of normal or mutant versions of nucleic acids encoding a CARD-containing polypeptide by microinjection, retroviral infection or other means well known to those skilled in the art, into appropriate fertilized embryos to produce a transgenic animal, see, for example, Hogan et al., Manipulating the Mouse Embryo: A Laboratory Manual (Cold Spring Harbor Laboratory, (1986)). Transgenic animal model systems are useful for in vivo screening of compounds for identification of specific ligands, such as agonists or antagonists, which activate or inhibit a biological activity.

Also contemplated herein, is the use of homologous recombination of mutant or normal versions of CARD-encoding genes with the native gene locus in transgenic animals, to alter the regulation of expression or the structure of CARD-containing polypeptides by replacing the endogeneous gene with a recombinant or mutated CARD-encoding gene. Methods for producing a transgenic non-human mammal including a gene knock-out non-human mammal, are well known to those skilled in the art (see, Capecchi et al., Science 244:1288 (1989); Zimmer et al., Nature 338:150 (1989); Shastry, Experentia, 51:1028-1039 (1995); Shastry, Mol. Cell. Biochem., 181:163-179 (1998); and U.S. Pat. No. 5,616,491, issued Apr. 1, 1997, No. 5,750,826, issued May 12, 1998, and No. 5,981,830, issued Nov. 9, 1999).

In addition to homologous recombination, additional methods such as microinjection can be used which add genes to the host genome without removing host genes. Microinjection can produce a transgenic animal that is capable of expressing both endogenous and exogenous CARD-containing polypeptides. Inducible promoters can be linked to the coding region of nucleic acids to provide a means to regulate expression of the transgene. Tissue specific regulatory elements can be linked to the coding region to permit tissue-specific expression of the transgene. Transgenic animal model systems are useful for in vivo screening of compounds for identification of specific ligands, i.e., agonists and antagonists, which activate or inhibit CARD-containing polypeptide responses.

In accordance with another embodiment of the invention, a method is provided for identifying a CARD-binding molecule, such as a CARD-associated polypeptide (CAP). The method is carried out by contacting an invention CARD-containing polypeptide with a candidate CARD-binding molecule, such as a candidate CAP, and detecting association of the CARD-containing polypeptide with the molecule.

As used herein, the term “CARD-binding molecule” means a molecule that can specifically bind to the CARD-containing polypeptides of the invention, or to any functional fragment of a CARD-containing polypeptide of the invention. A CARD-binding molecule can be a naturally occurring macromolecule, such as a peptide, nucleic acid, carbohydrate, lipid, or any combination thereof. A CARD-binding molecule also can be a partially or completely synthetic derivative, analog or mimetic of such a macromolecule, or a small organic or inorganic molecule prepared partly or completely by combinatorial chemistry methods. A CARD-binding molecule further can be an antibody, including a monoclonal, humanized and chimeric antibodies, and functional fragments of an antibody includes chimeric, bifunctional, humanized and single chain antibodies (scFv), variable region fragments (Fv or Fd), Fab and F(ab)₂. Such an antibody can be naturally occurring or non-naturally occurring.

As used herein, the term “NACHT-binding molecule” means a molecule that can specifically bind to the NAHCT domain of a CARD-containing polypeptide of the invention, or to any functional fragment of a CARD-containing polypeptide of the invention that contains a NACHT domain. A NACHT-binding molecule can be a naturally occurring macromolecule, such as a peptide, nucleic acid, carbohydrate, lipid, or any combination thereof. A NACHT-binding molecule also can be a partially or completely synthetic derivative, analog or mimetic of such a macromolecule, or a small organic or inorganic molecule prepared partly or completely by combinatorial chemistry methods. A NACHT-binding molecule further can be an antibody, including a monoclonal, humanized and chimeric antibodies, and functional fragments of an antibody includes chimeric, bifunctional, humanized and single chain antibodies (scFv), variable region fragments (Fv or Fd), Fab and F(ab)₂. Such an antibody can be naturally occurring or non-naturally occurring.

In the methods of the invention for identifying a CARD-binding molecule or NACHT-binding molecule, association of the CARD-containing or NACHT-containing polypeptide with a binding molecule can be detected using a variety of methods. For example, a scintillation proximity assay (Alouani, Methods Mol. Biol. 138:135-41 (2000)) can be used. Scintillation proximity assays involve the use of a fluomicrosphere coated with an acceptor molecule, such as an antibody, to which an antigen will bind selectively in a reversible manner. For example, a CARD- or NACHT-containing polypeptide can be bound to a fluomicrosphere using an antibody that specifically binds to the polypeptide, and contacted with a ³H or ¹²⁵I labeled candidate binding molecule. If the labeled candidate binding molecule specifically binds to the CARD- or NACHT-containing polypeptide, the radiation energy from the labeled candidate binding molecule is absorbed by the fluomicrosphere, thereby producing light which is easily measured.

Additional assays suitable for identifying a CARD-binding molecule or NACHT-binding molecule can include, without limitation, UV or chemical cross-linking assays (Fancy, Curr. Opin. Chem. Biol. 4:28-33 (2000)) and biomolecular interaction analyses (Weinberger et al., Pharmacogenomics 1:395-416 (2000)). Specific binding of a candidate binding molecule to a CARD- or NACHT-containing polypeptide can be determined by cross-linking these two components, if they are in contact with each other, using UV or a chemical cross-linking agent. In addition, a biomolecular interaction analysis (BIA) can detect whether two components are in contact with each other. In such an assay, one component, such as a CARD- or NACHT-containing polypeptide is bound to a BIA chip, and a second component such as a candidate binding molecule is passed over the chip. If the candidate binding molecule binds to the CARD- or NACHT-containing polypeptide, the contact results in an electrical signal, which is readily detected.

Further assays suitable for identifying a CARD-binding molecule or NACHT-binding molecule include those based on NMR methods. Such methods take advantage of the significant perturbations that can be observed in NMR-sensitive parameters of a candidate binding molecule or its target, such as a CARD- or NACHT-containing molecule or domain thereof, that occur upon complex formation between the candidate binding molecule and target polypeptide. These perturbations can be used to detect binding between a candidate binding molecule and CARD- or NACHT-containing polypeptide, as well as to assess the strength of the binding interaction. In addition, some NMR techniques allow the identification of the binding site or part of the binding molecule responsible for interacting with the CARD- or NACHT-containing polypeptide. Exemplary NMR methods useful for identifying a CARD-binding molecule or NACHT-binding molecule include “SAR by NMR,” which is described, for example, in Shuker et al. Science, 274, 1531-1534 (1996), and a variety of NMR-based screening assays, including SHAPES screening, fragment-based approaches for lead optimization using NMR, and fluorine-NMR competition binding experiments, all of which are described, for example, in Combinatorial Chemistry & High Throughput Screening, Vol. 5, No. 8 (2002) and in Hajduk et al., Quarterly Reviews of Biophysics 32(3):211-240 (1999).

Fluorescence-based assays are also suitable for identifying a CARD-binding molecule or NACHT-binding molecule. Examples of fluorescence methods applicable to determining an interaction between a candidate binding molecule and the CARD- or NACHT-containing polypeptide include observations fluorescence intensity changes resulting from an alteration in interaction between binding molecule and target polypeptide; fluorescence resonance energy transfer (FRET), which is useful for determining change in fluorescence intensity based on distance between binding molecule and target polypeptide; fluorescence polarization changes resulting a change in size of an observed binding partner when associated or dissociated from the another binding partner; fluorescence lifetime changes, and fluorescence correlation spectroscopy, which are based on translation diffusion, a parameter related to the size of an observed binding partner.

Such methods can involve employing a fluorescently labeled candidate binding molecule or fluorescently labeled target polypeptide. For example, a fluorophore can be detected based on the excitation or emission wavelengths of the fluorophore, fluorescence polarization of the fluorophore, or intensity of fluorescence emitted from the fluorophore. Alternatively, detection can be based on a difference in a measurable property of the label for the bound and unbound state. Other measurable differences that can be used to determine association of a fluorophore-labeled candidate binding molecule with a CARD- or NACHT-containing polypeptide, for example, different emission intensity due to the presence or absence of a quenching agent, difference in emission wavelength due to the presence or absence of a fluorescence resonance energy transfer (FRET) donor or acceptor, or difference in emission wavelength due to differences in fluorophore conformation or environment. A labeled binding molecule that is bound to a CARD- or NACHT-containing polypeptide can be detected according to a known measurable property of the label. Dissociation of a labeled candidate binding molecule from the CARD- or NACHT-containning polypeptide can be detected as absence or reduction in the amount of label from the polypeptide in the presence of a competitive binding molecule or as a reversal of a change that occurs upon association of the labeled candidate binding molecule with the CARD- or NACHT-containing polypeptide in the presence of a competitive binding candidate binding molecule.

In addition, virtual computational methods can be used to identify a CARD-binding molecule or NACHT-binding molecule. Exemplary virtual computational methodology involves virtual docking of small-molecule binding molecules on a virtual representation of the structure of a CARD-or NACHT-containing polypeptide or fragment in order to determine or predict specific binding. See, for example, Shukur et al., supra, 1996; Lengauer et al., Current Opinions in Structural Biology 6:402-406 (1996); Choichet et al., Journal of Molecular Biology 221:327-346 (1991); Cherfils et al., Proteins 11:271-280 (1991); Palma et al., Proteins 39:372-384 (2000); Eckert et al., Cell 99:103-115 (1999); Loo et al., Med. Res. Rev. 19:307-319 (1999); Kramer et al., J. Biol. Chem. (2000).

As used herein, the term “CARD-associated polypeptide” or “CAP” means a polypeptide that can specifically bind to the CARD-containing polypeptides of the invention, or to any functional fragment of a CARD-containing polypeptide of the invention. Because CARD-containing polypeptides of the invention contain domains which can self-associate, CARD-containing polypeptides are encompassed by the term CAP. An exemplary CAP is a protein or a polypeptide portion of a protein that can bind a NACHT, CARD, LRR or ANGIO-R domain of an invention CARD-containing polypeptide.

As used herein, the term “NACHT-associated polypeptide” or “NAP” means a polypeptide that can specifically bind to the NACHT domain of the CARD-containing polypeptides of the invention, or to a functional fragment of a CARD-containing polypeptide of the invention that contains a NACHT domain.

A CAP or NAP can be identified, for example, using in vitro protein binding assays similar to those described in, for example, Ausubel et al., supra, 2000, and by in vivo methods including methods such as yeast two-hybrid assays, or other protein-interaction assays and methods known in the art.

Normal association of CARD-containing polypeptide and a CAP or NAP polypeptide in a cell can be altered due, for example, to the expression in the cell of a variant CAP or CARD-containing polypeptide, respectively, either of which can compete with the normal binding function of a CARD-containing polypeptide and, therefore, can decrease the association of CAP or NAP and CARD-containing polypeptides in a cell. The term “variant” is used generally herein to mean a polypeptide that is different from the CAP, NAP or CARD-containing polypeptide that normally is found in a particular cell type. Thus, a variant can include a mutated protein or a naturally occurring protein, such as an isoform, that is not normally found in a particular cell type.

CARD-containing polypeptides and CARD- or NACHT-associated polypeptides of the invention can be characterized, for example, using in vitro binding assays or the yeast two hybrid system. An in vivo transcription activation assay such as the yeast two hybrid system is particularly useful for identifying and manipulating the association of proteins. In addition, the results observed in such an assay likely mirror the events that naturally occur in a cell. Thus, the results obtained in such an in vivo assay can be predictive of results that can occur in a cell in a subject such as a human subject.

A transcription activation assay such as the yeast two hybrid system is based on the modular nature of transcription factors, which consist of functionally separable DNA-binding and trans-activation domains. When expressed as separate proteins, these two domains fail to mediate gene transcription. However, transcription activation activity can be restored if the DNA-binding domain and the trans-activation domain are bridged together due, for example, to the association of two proteins. The DNA-binding domain and trans-activation domain can be bridged, for example, by expressing the DNA-binding domain and trans-activation domain as fusion proteins (hybrids), provided that the proteins that are fused to the domains can associate with each other. The non-covalent bridging of the two hybrids brings the DNA-binding and trans-activation domains together and creates a transcriptionally competent complex. The association of the proteins is determined by observing transcriptional activation of a reporter gene.

The yeast two hybrid systems exemplified herein use various strains of S. cerevisiae as host cells for vectors that express the hybrid proteins. A transcription activation assay also can be performed using, for example, mammalian cells. However, the yeast two hybrid system is particularly useful due to the ease of working with yeast and the speed with which the assay can be performed. For example, yeast host cells containing a lacZ reporter gene linked to a LexA operator sequence can be used to demonstrate that a CARD domain of an invention CARD-containing polypeptide can interact with itself or other CARD-containing polypeptides. For example, the DNA-binding domain can consist of the LexA DNA-binding domain, which binds the LexA promoter, fused to the CARD domain of a CARD-containing polypeptide of the invention and the trans-activation domain can consist of the B42 acidic region separately fused to several cDNA sequences which encode known CARD-containing polypeptides. When the LexA domain is non-covalently bridged to a trans-activation domain fused to a CARD-containing polypeptide, the association can activate transcription of the reporter gene.

A CAP, for example, a CARD-containing polypeptide, a NACHT-containing polypeptide or a LRR-containing polypeptide, also can be identified using well known in vitro assays, for example, an assay utilizing a glutathione-S-transferase (GST) fusion protein. Such an in vitro assay provides a simple, rapid and inexpensive method for identifying and isolating a CAP. Such an in vitro assay is particularly useful in confirming results obtained in vivo and can be used to characterize specific binding domains of a CAP. For example, a GST can be fused to a CARD-containing polypeptide of the invention, and expressed and purified by binding to an affinity matrix containing immobilized glutathione. If desired, a sample that can contain a CAP or active fragments of a CAP can be passed over an affinity column containing bound GST/CARD and a CAP that binds to a CARD-containing polypeptide can be obtained. In addition, GST/CARD can be used to screen a cDNA expression library, wherein binding of the GST/CARD fusion protein to a clone indicates that the clone contains a cDNA encoding a CAP.

Thus, one of skill in the art will recognize that using the CARD-containing polypeptides described herein, a variety of methods, such as protein purification, protein interaction cloning, or protein mass-spectrometry, can be used to identify a CAP.

Although the terms “CAP” and “NAP” are used generally, it should be recognized that a CAP or NAP that is identified using the novel polypeptides described herein can be a fragment of a protein. Thus, as used herein, a CAP also includes a polypeptide that specifically associates to a portion of an invention CARD-containing polypeptide that does not include a CARD domain. For example, a CAP can associate with the NACHT domain of CLAN or CARD3X. A CAP that can associate with the NACHT domain of a CLAN or CARD3X also if referred to herein as a “NAP.” As used herein, a “candidate CAP” or “candidate NAP” refers to a polypeptide containing a polypeptide sequence know or suspected of binding one or more CARD-containing polypeptides of the invention. Thus, a CAP or NAP can represent a full-length protein or a CARD-associating fragment thereof. Since a CAP or NAP polypeptide can be a full-length protein or a CARD-associating fragment thereof, one of skill in the art will recognize that a CAP-encoding nucleic acid, such as the genomic sequence, an mRNA sequence or a cDNA sequence need not encode the full-length protein. Thus, a cDNA can encode a polypeptide that is a fragment of a full-length CAP or NAP which, nevertheless, binds one or more invention CARD-containing polypeptides. It is also within the scope of the invention that a full-length CAP or NAP can assume a conformation that does not, absent some post-translational modification, bind a CARD-containing polypeptide of the invention, due, for example, to steric blocking of the binding site. Thus, a CAP or NAP can be a protein or a polypeptide portion of a protein that can bind one of the CARD-containing polypeptides of the invention. Also, it should be recognized that a CAP or NAP can be identified by using a minimal polypeptide derived from the sequences of the CARD-containing polypeptides of the invention, and does not necessarily require that the full-length molecules be employed for identifying such CAPs and NAPs.

Since CARD-containing polypeptides can be involved in apoptosis, the association of a CAP or NAP with a CARD-containing polypeptide can affect the sensitivity or resistance of a cell to apoptosis or can induce or block apoptosis induced by external or internal stimuli. The identification of various CAPs and NAPs by use of known methods can be used to determine the function of these CAPs or NAPs in cell death or signal transduction pathways controlled by CARD-containing polypeptides, allowing for the development of assays that are useful for identifying agents that effectively alter the association of a CAP with a CARD-containing polypeptide or a NAP with a NACHT-containing polypeptide. Such agents can be useful for providing effective therapy for conditions caused, at least in part, by insufficient apoptosis, such as a cancer, autoimmune disease or certain viral infections. Such agents can also be useful for providing an effective therapy for diseases where excessive apoptosis is known to occur, such as stroke, heart failure, or AIDS.

Assays of the invention can be used for identification of agents that alter the self-association of the CARD-containing polypeptides of the invention. Thus, the methods of the invention can be used to identify agents that alter the self-association of CARD3X-2 (set forth in SEQ ID NO:197) via its CARD domains, NACHT domains, LRR domains, or other domains within this polypeptide.

The ATP-binding and hydrolysis of the NACHT domains can be critical for function of a NAC polypeptide, for example, by altering the oligomerization of the NAC. Thus, agents that interfere with or enhance ATP or nucleotide binding and/or hydrolysis by the NACHT domain of a NAC polypeptide of the invention, such as CARD3X-2 (SEQ ID NO:197), can also be useful for altering the activity of these polypeptides in cells.

A further embodiment of the invention provides a method to identify agents that can effectively alter CARD-containing polypeptide activity, for example the ability of CARD-containing polypeptides to associate with one or more heterologous proteins. Thus, the present invention provides a screening assay useful for identifying an effective agent, which can alter the association of a CARD-containing polypeptide with a CARD-associated polypeptide (CAP), such as a heterologous CARD-containing polypeptide. Since CARD-containing polypeptides are involved in biochemical processes such as apoptosis, the identification of such effective agents can be useful for altering the level of a biochemical process such as apoptosis in a cell, for example in a cell of a subject having a pathology characterized by an increased or decreased level of apoptosis.

Further, effective agents can be useful for alteration of other biochemical process modulated by a CARD-containing polypeptide of the invention. Additional biochemical processes modulated by CARD-containing polypeptide include, for example, NF-kB induction, cytokine processing, cytokine receptor signaling, cJUN N-terminal kinase induction, and caspase-mediated proteolysis activation/inhibition, transcription, inflammation and cell adhesion.

As used herein, the term “agent” means a chemical or biological molecule such as a simple or complex organic molecule, a peptide, a peptido-mimetic, a polypeptide, a protein or an oligonucleotide that has the potential for altering the association of a CARD-containing polypeptide with a heterologous protein or altering the ability of a CARD-containing polypeptide to self-associate or altering the ligand binding or catalytic activity of a CARD-containing polypeptide. The term also refers to a chemical or biological molecule such as a simple or complex organic molecule, a peptide, a peptido-mimetic, a polypeptide, a protein or an oligonucleotide that has the potential for altering the association of a NACHT-containing polypeptide with a heterologous NACHT-containing protein or altering the ability of a NACHT-containing polypeptide to self-associate or altering the ligand binding or catalytic activity of a NACHT-containing polypeptide. An exemplary ligand binding activity is nucleotide binding activity, such as ADP or ATP binding activity; and exemplary catalytic activities are nucleotide hydrolytic activity and proteolytic activity.

The term “effective agent” is used herein to mean an agent that is confirmed as capable of altering the association of a CARD-containing polypeptide with a heterologous protein or altering the ability of a CARD-containing polypeptide to self-associate or altering the ligand binding or catalytic activity of a CARD-containing polypeptide. For example, an effective agent may be an anti-CARD antibody, a CARD-associated polypeptide, a caspase inhibitor, and the like. In addition, the term “effective agent” is used herein to mean an agent that is confirmed as capable of altering the association of a NACHT-containing polypeptide with another NACHT-containing polypeptide or altering the ability of a NACHT-containing polypeptide to self-associate. For example, an effective agent may be an anti-NACHT antibody, a NACHT-associated polypeptide, and the like.

As used herein, the term “alter the association” means that the association between two specifically interacting polypeptides either is increased or decreased due to the presence of an effective agent. As a result of an altered association of CARD-containing polypeptide or NACHT-containing polypeptide with another polypeptide in a cell, the activity of the CARD-containing polypeptide, the CAP, the NACHT-containing polypeptide or the NAP, can be increased or decreased, thereby altering a biochemical process, for example, the level of apoptosis or immune signaling response in the cell. As used herein, the term “alter the activity” means that the agent can increase or decrease the activity of a CARD-containing polypeptide or NACHT-containing polypeptide in a cell, thereby modulating a biochemical process in a cell, for example, the level of apoptosis in the cell. Similarly, the term “alter the level” of a biological process modulated by a CARD-containing polypeptide refers to an increase or decrease a biochemical process which occurs upon altering the activity of a CARD-containing polypeptide. For example, an effective agent can increase or decrease the CARD:CARD-associating activity of a CARD-containing polypeptide, which can result in decreased apoptosis. In another example, alteration of the ATP hydrolysis activity can modulate the ability of the NACHT domain of a CARD-containing polypeptide to associate with other NACHT-containing polypeptides, such as Apaf-1, thereby altering any process effected by such association between a CARD-containing polypeptide and a NACHT-containing polypeptide. The term “alter the level” of a biological process modulated by a NACHT-containing polypeptide refers to an increase or decrease a biochemical process which occurs upon altering the activity of a NACHT-containing polypeptide. For example, an effective agent can increase or decrease the NACHT:NACHT-associating activity of a NACHT-containing polypeptide, which can result in decreased apoptosis or alteration in another cellular response, such as an inflammatory or innate immune system response.

An effective agent can act by interfering with the ability of a CARD-containing polypeptide or NACHT-containing polypeptide to associate with another polypeptide, or can act by causing the dissociation of a CARD- or NACHT-containing polypeptide from a complex with a CARD-associated polypeptide or NACHT-associated polypeptide, wherein the ratio of bound CARD-or NACHT containing polypeptide to free CARD- or NACHT containing polypeptide is related to the level of a biochemical process, such as, apoptosis, in a cell. For example, binding of a ligand to a CAP can allow the CAP, in turn, to bind a specific CARD-containing polypeptide such that all of the specific CARD-containing polypeptide is bound to a CAP, and can result in decreased apoptosis. Similarly, binding of a ligand to a NAP can allow the NAP, in turn, to bind a specific NACHT-containing polypeptide such that all of the specific NACHT-containing polypeptide is bound to a NAP, and can result in decreased apoptosis or other cellular function. The association, for example, of a CARD-containing polypeptide and a CARD-containing polypeptide can result in activation or inhibition of the NACHT:NACHT-associating activity of a CARD-containing polypeptide. In the presence of an effective agent, the association of a CARD-containing polypeptide and a CAP can be altered, which can, for example, alter the activation of caspases in the cell. As a result of the altered caspase activation, the level of apoptosis in a cell can be increased or decreased. Thus, the identification of an effective agent that alters the association of a CARD-containing polypeptide with another polypeptide can allow for the use of the effective agent to increase or decrease the level of a biological process such as apoptosis. In the presence of an effective agent, the association of a NACHT-containing polypeptide and a NAP can be altered, which can, for example, alter the activation of caspases in the cell. As a result of the altered caspase activation, the level of a biological process in a cell can be increased or decreased. Thus, the identification of an effective agent that alters the association of a NACHT-containing polypeptide with another polypeptide can allow for the use of the effective agent to increase or decrease the level of a biological process such as apoptosis or cellular signaling, for example, activation of NF-Kκ and production of IL-1β.

An effective agent can be useful, for example, to increase the level of apoptosis in a cell such as a cancer cell, which is characterized by having a decreased level of apoptosis as compared to its normal cell counterpart. An effective agent also can be useful, for example, to decrease the level of apoptosis in a cell such as a T lymphocyte in a subject having a viral disease such as acquired immunodeficiency syndrome, which is characterized by an increased level of apoptosis in an infected T cell as compared to a normal T cell. Thus, an effective agent can be useful as a medicament for altering the level of apoptosis in a subject having a pathology characterized by increased or decreased apoptosis. In addition, an effective agent can be used, for example, to decrease the level of apoptosis and, therefore, increase the survival time of a cell such as a hybridoma cell in culture. The use of an effective agent to prolong the survival of a cell in vitro can significantly improve bioproduction yields in industrial tissue culture applications. An effective agent also can be useful, for example, to modulate inflammatory or innate immune responses by modulating the activation of NF-KB transcription factors and regulating the activation of caspase-1. Thus, an effective agent can be useful as a medicament for altering the level of immune response in a subject having a pathology characterized by unwanted immune response or deficient immune response.

A CARD-containing polypeptide that lacks the ability to bind the NACHT domain or LRR domain of another polypeptide but retains the ability to self-associate via its CARD domain or to bind to other CARD-containing polypeptides is an example of an effective agent, since the expression of a non-NACHT-associating or non-catalytically active CARD-containing polypeptide in a cell can alter the association of a the endogenous CARD-containing polypeptide with itself or with CAPs.

Thus, it should be recognized that a mutation of a CARD-containing polypeptide can be an effective agent, depending, for example, on the normal levels of CARD-containing polypeptide and CARD-associated polypeptide that occur in a particular cell type. In addition, an active fragment of a CARD-containing polypeptide can be an effective agent, provided the active fragment can alter the association of a CARD-containing polypeptide and another polypeptide in a cell. Such active fragments, which can be peptides as small as about five amino acids, can be identified, for example, by screening a peptide library (see, for example, Ladner et al., U.S. Pat. No. 5,223,409) to identify peptides that can bind a CARD-associated polypeptide.

Similarly, a fragment of a CARD-associated polypeptide also can be an effective agent. A fragment of CARD-associated polypeptide can be useful, for example, for decreasing the association of a CARD-containing polypeptide with a CAP in a cell by competing for binding to the CARD-containing polypeptide. A non-naturally occurring peptido-mimetic also can be useful as an effective agent. Such a peptido-mimetic can include, for example, a peptoid, which is peptide-like sequence containing N-substituted glycines, or an oligocarbamate. A peptido-mimetic can be particularly useful as an effective agent due, for example, to having an increased stability to enzymatic degradation in vivo.

In accordance with another embodiment of the present invention, there is provided a method of identifying an effective agent that alters the association of an invention CARD-containing polypeptide with a CARD-associated polypeptide (CAP), by the steps of: (a) contacting a CARD-containing polypeptide and a CAP polypeptide, under conditions that allow the CARD-containing polypeptide and CAP polypeptide to associate, with an agent suspected of being able to alter the association of the CARD-containing polypeptide and CAP polypeptides; and (b) detecting the altered association of the CARD-containing polypeptide and CAP polypeptide, where the altered association identifies an effective agent.

Also provided by the invention is a method for identifying an effective agent that alters association of a NACHT-containing polypeptide with a NACHT-associated polypeptide (NAP). The method involves (a) contacting a NACHT-containing polypeptide selected from SEQ ID NOS: 188, 189 and 197, and the NAP with an agent suspected of being able to alter the association of the NACHT-containing polypeptide and the NAP, under conditions that allow association between the NACHT-containing polypeptide and the NAP; and (b) detecting the altered association of the NACHT-containing polypeptide and the NAP, wherein the altered association identifies an effective agent. In an embodiment, the NAP is selected from CARD3X, CARD3X-2, Nod1, NAC, PAN2, NAIP and cyropyrin.

Methods well-known in the art for detecting the altered association of the CARD-containing polypeptide and CAP polypeptides, or detecting the altered association of the NACHT-containing polypeptide and NAP polypeptide, for example, measuring protein:protein binding, protein degradation or apoptotic activity can be employed in bioassays described herein to identify agents as agonists or antagonists of CARD- or NACHT containing polypeptides. As described herein, CARD-containing polypeptides and NACHT-containing polypeptides have the ability to self-associate. Thus, methods for identifying effective agents that alter the association of a CARD-containing polypeptide with a CAP are useful for identifying effective agents that alter the ability of a CARD-containing polypeptide to self-associate. Similarly, methods for identifying effective agents that alter the association of a NACHT-containing polypeptide with a NAP are useful for identifying effective agents that alter the ability of the NACHT-containing polypeptide to self-associate. It is understood that a NACHT-containing polypeptide can also be a CARD-containing polypeptide.

As is described herein, the NACHT domain of CLANA is capable of homotypic NACHT:NACHT interactions as well as heterotypic NACHT:NACHT interactions with a variety of heterologous NACHT domains, including those of CARD3X, CARD3X-2, Nod1, NAC, PAN2, NAIP and cyropyrin. Therefore, the methods of the invention can be used to identify an effective agent that alters association of a NACHT-containing polypeptide, such as CLAN, CARD3X or CARD3X-2, with a NACHT-associated polypeptide (NAP), such as CLAN-A, CARD3X, CARD3X-2, Nod1, NAC, PAN2, NAIP and cyropyrin, as well as a varity of other polypeptides that contain a NACHT domain. A NACHT domain-containing polypeptide for use in the methods of the invention can be obtained using a variety of procedures, including biochemical purification and recombinant expression methods well known to those skilled in the art. The amino acid sequence of human Nod1 and encoding nucleotide sequence are referenced in GenBank, for example, as Accession No. AF113925; the amino acid sequence of human NAC and encoding nucleotide sequence are referenced in GenBank, for example, as Accession No. NM_{—}033007; the amino acid sequence of human PAN2 and encoding nucleotide sequence are referenced in GenBank, for example, as Accession No. AY072792; the amino acid sequence of human NAIP and encoding nucleotide sequence are referenced in GenBank, for example, as Accession Nos. NM_{—}004536 and NP_{—}004527.1, respectively; the amino acid sequence of cyropyrin and encoding nucleotide sequence are referenced in GenBank, for example, as Accession No. AY092033. Those skilled in the art will recognize that public databases, including GenBank, contain other nucleotide and amino acid sequence entries for each of the above-listed NACHT-containing polypeptides, some of which can contain minor differences with respect to the above-listed accession numbers. Any of such nucleotide and amino acid entries corresponding to a NACHT-containing polypeptide can be employed for preparing a polypeptide for use in the methods of the invention, so long as the NACHT domain of the polypeptide maintains the ability to interact with a respective NACHT domain binding partner. Additional NACHT domain-containing polypeptides can be identified, for example, by searching public or private databases using a NACHT domain motif identifying algorithm. Methods for performing such searches are well known to those skilled in the art.

As used herein, “conditions that allow said CARD-containing polypeptide and CAP polypeptide to associate” and “conditions that allow association between the NACHT-containing polypeptide and the NAP” refers to environmental conditions in which a CARD-containing polypeptide and CAP, or NACHT-containing polypeptide and NAC, can specifically associate. Such conditions will typically be aqueous conditions, with a pH between 3.0 and 11.0, and temperature below 100° C. Preferably, the conditions will be aqueous conditions with salt concentrations below the equivalent of 1 M NaCl, and pH between 5.0 and 9.0, and temperatures between 0° C. and 50° C. Most preferably, the conditions will range from physiological conditions of normal yeast or mammalian cells, or conditions favorable for carrying out in vitro assays such as immunoprecipitation and GST protein:protein association assays, and the like.

In another embodiment of the invention, a method is provided for identifying agents that modulate a ligand binding or catalytic activity of an invention CARD-containing polypeptide. The method contains the steps of contacting an invention CARD-containing polypeptide with an agent suspected of modulating a ligand binding or catalytic activity of the CARD-containing polypeptide and measuring a ligand binding or catalytic activity of the CARD-containing polypeptide, where modulated ligand binding or catalytic activity identifies the agent as an agent that alters the ligand binding or catalytic activity of a CARD-containing polypeptide.

As used herein in regard to ligand binding or catalytic activity, “modulate” refers to an increase or decrease in ligand binding or catalytic activity. Thus, modulation encompasses inhibition of ligand binding or catalytic activity as well as activation or enhancement of ligand binding or catalytic activity. Exemplary ligand binding activities include nucleotide binding activity. Exemplary catalytic binding activities include nucleotide hydrolysis and proteolysis activities.

Methods for measuring ligand binding or catalytic activities are well known in the art, as disclosed herein. For example, an agent known or suspected of modulating ligand binding or catalytic activity can be contacted with an invention CARD-containing polypeptide in vivo or in vitro, and the ligand binding or catalytic activity can be measured using known methods. For example, enzymatic activity can be measured using a cleavable reporter, where the cleavable reporter generates or alters a measurable signal such as absorption, fluorescence or radioactive decay. Exemplary agents that can modulate ligand binding or catalytic activity include peptides, peptidomimetics and other peptide analogs, non-peptide organic molecules such as naturally occuring protease inhibitors and derviatives thereof, nucleotides and nucleotide analogs, and the like. Such inhibitors can be either reversible or irreversible, as is well known in the art.

Agents that modulate the ligand binding or catalytic activity of a CARD-containing polypeptide identified using the invention methods can be used to modulate the activity of a CARD-containing polypeptide. For example, and agent can modulate the nucleotide binding or nucleotide hydrolytic activity of a NACHT domain of a CARD-containing polypeptide. In another example, an agent can modulate the catalytic activity of a protease domain such as a caspase domain. Methods of modulating the ligand binding or catalytic activities of invention CARD-containing proteins can be used in methods of altering biochemical processes modulated by CARD-containing proteins, such as the biochemical processes disclosed herein.

In yet another embodiment of the present invention, there are provided methods for altering ligand binding or catalytic activity of a CARD-containing polypeptide of the invention, the method comprising contacting an CARD-containing polypeptide with an effective amount of an agent identified by the herein-described bioassays.

The present invention also provides in vitro screening assays. Such screening assays are particularly useful in that they can be automated, which allows for high through-put screening, for example, of randomly or rationally designed agents such as drugs, peptidomimetics or peptides in order to identify those agents that effectively alter the association of a CARD-containing polypeptide and a CAP or the catalytic or ligand binding activity of a CARD-containing polypeptide and, thereby, alter a biochemical process modulated by a CARD-containing polypeptide such as apoptosis. An in vitro screening assay can utilize, for example, a CARD-containing polypeptide including a CARD-containing fusion protein such as a CARD-glutathione-S-transferase fusion protein. For use in the in vitro screening assay, the CARD-containing polypeptide should have an affinity for a solid substrate as well as the ability to associate with a CARD-associated polypeptide. For example, when a CARD-containing polypeptide is used in the assay, the solid substrate can contain a covalently attached anti-CARD antibody. Alternatively, a GST/CARD fusion protein can be used in the assay and the solid substrate can contain covalently attached glutathione, which is bound by the GST component of the GST/CARD fusion protein. Similarly, a CARD-associated polypeptide, or GST/NACHT-containing polypeptide fusion protein can be used in any of a variety of in vitro enzymatic or in vitro binding assays known in the art and described in texts such as Ausubel et al., supra, 2000.

An in vitro screening assay can be performed by allowing a CARD-containing polypeptide, for example, to bind to the solid support, then adding a CARD-associated polypeptide and an agent to be tested. Reference reactions, which do not contain an agent, can be performed in parallel. Following incubation under suitable conditions, which include, for example, an appropriate buffer concentration and pH and time and temperature that permit binding of the particular CARD-containing polypeptide and CARD-associated polypeptide, the amount of protein that has associated in the absence of an agent and in the presence of an agent can be determined. The association of a CARD-associated polypeptide with a CARD-containing polypeptide can be detected, for example, by attaching a detectable moiety such as a radionuclide or a fluorescent label to a CARD-associated polypeptide and measuring the amount of label that is associated with the solid support, wherein the amount of label detected indicates the amount of association of the CARD-associated polypeptide with a CARD-containing polypeptide. An effective agent is determined by comparing the amount of specific binding in the presence of an agent as compared to a reference level of binding, wherein an effective agent alters the association of CARD-containing polypeptide with the CARD-associated polypeptide. Such an assay is particularly useful for screening a panel of agents such as a peptide library in order to detect an effective agent.

Various binding assays to identify cellular proteins that interact with protein binding domains are known in the art and include, for example, yeast two-hybrid screening assays (see, for example, U.S. Pat. Nos. 5,283,173, 5,468,614 and 5,667,973; Ausubel et al., supra, 2000; Luban et al., Curr. Opin. Biotechnol. 6:59-64 (1995)) and affinity column chromatography methods using cellular extracts. By synthesizing or expressing polypeptide fragments containing various CARD-associating sequences or deletions, the CARD binding interface can be readily identified.

Another assay for screening of agents that alter the activity of a CARD-containing polypeptide is based on altering the phenotype of yeast by expressing a CARD-containing polypeptide. In one embodiment, expression of a CARD-containing polypeptide can be inducible (Tao et al., J. Biol. Chem. 273:23704-23708 (1998), and the compounds can be screened when CARD-containing polypeptide expression is induced. CARD-containing polypeptides of the invention can also be co-expressed in yeast with CAP polypeptides used to screen for compounds that antagonize the activity of the CARD-containing polypeptide.

Also provided with the present invention are assays to identify agents that alter CARD-containing polypeptide expression. Methods to determine CARD-containing polypeptide expression can involve detecting a change in CARD-containing polypeptide abundance in response to contacting the cell with an agent that modulates CARD-containing polypeptide expression. Assays for detecting changes in polypeptide expression include, for example, immunoassays with CARD-specific antibodies, such as immunoblotting, immunofluorescence, immunohistochemistry and immunoprecipitation assays, as described herein.

As understood by those of skill in the art, assay methods for identifying agents that alter CARD-containing polypeptide activity generally require comparison to a reference. One type of a “reference” is a cell or culture that is treated substantially the same as the test cell or test culture exposed to the agent, with the distinction that the “reference” cell or culture is not exposed to the agent. Another type of “reference” cell or culture can be a cell or culture that is identical to the test cells, with the exception that the “reference” cells or culture do not express a CARD-containing polypeptide. Accordingly, the response of the transfected cell to an agent is compared to the response, or lack thereof, of the “reference” cell or culture to the same agent under the same reaction conditions.

Methods for producing pluralities of agents to use in screening for compounds that alter the activity of a CARD-containing polypeptide, including chemical or biological molecules such as simple or complex organic molecules, metal-containing compounds, carbohydrates, peptides, proteins, peptidomimetics, glycoproteins, lipoproteins, nucleic acids, antibodies, and the like, are well known in the art and are described, for example, in Huse, U.S. Pat. No. 5,264,563; Francis et al., Curr. Opin. Chem. Biol. 2:422-428 (1998); Tietze et al., Curr. Biol., 2:363-371 (1998); Sofia, Mol. Divers. 3:75-94 (1998); Eichler et al., Med. Res. Rev. 15:481-496 (1995); and the like. Libraries containing large numbers of natural and synthetic agents also can be obtained from commercial sources. Combinatorial libraries of molecules can be prepared using well known combinatorial chemistry methods (Gordon et al., J. Med. Chem. 37: 1233-1251 (1994); Gordon et al., J. Med. Chem. 37: 1385-1401 (1994); Gordon et al., Acc. Chem. Res. 29:144-154 (1996); Wilson and Czarnik, eds., Combinatorial Chemistry: Synthesis and Application, John Wiley & Sons, New York (1997)).

The invention further provides a method of diagnosing or predicting clinical prognosis of a pathology characterized by an increased or decreased level of a CARD-containing polypeptide in a subject. The method includes the steps of (a) obtaining a test sample from the subject; (b) contacting the sample with an agent that can bind a CARD-containing polypeptide of the invention under suitable conditions, wherein the conditions allow specific binding of the agent to the CARD-containing polypeptide; and (c) comparing the amount of the specific binding in the test sample with the amount of specific binding in a reference sample, wherein an increased or decreased amount of the specific binding in the test sample as compared to the reference sample is diagnostic of, or predictive of the clinical prognosis of, a pathology. The agent can be, for example, an anti-CARD antibody, a CARD-associated-polypeptide (CAP), or a CARD-encoding nucleic acid.

Exemplary pathologies for diagnosis or the prediction of clinical prognosis include any of the pathologies described herein, such as neoplastic pathologies (e.g. cancer), autoimmune diseases; pathologies related to abnormal cell proliferation or abnormal cell death (e.g. apoptosis); and pathologies relating to abnormal immune response, including undesired or deficient innate immune response, as disclosed herein.

The invention also provides a method of diagnosing cancer or monitoring cancer therapy by contacting a test sample from a patient with a CARD-specific antibody. The invention additionally provides a method of assessing prognosis (e.g., predicting the clinical prognosis) of patients with cancer comprising contacting a test sample from a patient with a CARD-specific antibody.

The invention additionally provides a method of diagnosing cancer or monitoring cancer therapy by contacting a test sample from a patient with a oligonucleotide that selectively hybridizes to a CARD-encoding nucleic acid molecule. The invention further provides a method of assessing prognosis (e.g., predicting the clinical prognosis) of patients with cancer by contacting a test sample from a patient with a oligonucleotide that selectively hybridizes to a CARD-encoding nucleic acid molecule.

The methods of the invention for diagnosing cancer or monitoring cancer therapy using a CARD-specific antibody or oligonucleotide or nucleic acid that selectively hybridizes to a CARD-encoding nucleic acid molecule can be used, for example, to segregate patients into a high risk group or a low risk group for diagnosing cancer or predicting risk of metastasis or risk of failure to respond to therapy. Therefore, the methods of the invention can be advantageously used to determine, for example, the risk of metastasis in a cancer patient, or the risk of an autoimmune disease of a patient, or as a prognostic indicator of survival or disease progression in a cancer patient or patient with an autoimmune disease. One of ordinary skill in the art would appreciate that the prognostic indicators of survival for cancer patients suffering from stage I cancer can be different from those for cancer patients suffering from stage IV cancer. For example, prognosis for stage I cancer patients can be oriented toward the likelihood of continued growth and/or metastasis of the cancer, whereas prognosis for stage IV cancer patients can be oriented toward the likely effectiveness of therapeutic methods for treating the cancer. Accordingly, the methods of the invention directed to measuring the level of or determining the presence of a CARD-containing polypeptide or CARD-encoding nucleic acid can be used advantageously as a prognostic indicator for the presence or progression of a cancer or response to therapy.

The invention further provides methods for introducing a CARD-encoding nucleic acid into a cell in a subject, for example, for gene therapy. Viruses are specialized infectious agents that can elude host defense mechanisms and can infect and propagate in specific cell types. Viral based systems provide the advantage of being able to introduce relatively high levels of the heterologous nucleic acid into a variety of cells. Suitable viral vectors for introducing an invention CARD-encoding nucleic acid into mammalian cells (e.g., vascular tissue segments) are well known in the art. These viral vectors include, for example, Herpes simplex virus vectors (e.g., Geller et al., Science, 241:1667-1669 (1988)), Vaccinia virus vectors (e.g., Piccini et al., Meth. in Enzymology, 153:545-563 (1987); Cytomegalovirus vectors (Mocarski et al., in Viral Vectors, Y. Gluzman and S. H. Hughes, Eds., Cold Spring Harbor Laboratory, Cold Spring Harbor, N.Y., 1988, pp. 78-84), Moloney murine leukemia virus vectors (Danos et al., Proc. Natl. Acad. Sci., USA, 85:6469 (1980)), adenovirus vectors (e.g., Logan et al., Proc. Natl. Acad. Sci., USA, 81:3655-3659 (1984); Jones et al., Cell, 17:683-689 (1979); Berkner, Biotechniques, 6:616-626 (1988); Cotten et al., Proc. Natl. Acad. Sci., USA, 89:6094-6098 (1992); Graham et al., Meth. Mol. Biol., 7:109-127 (1991)), adeno-associated virus vectors, retrovirus vectors (see, e.g., U.S. Pat. Nos. 4,405,712 and 4,650,764), and the like. Especially preferred viral vectors are the adenovirus and retroviral vectors.

Suitable retroviral vectors for use herein are described, for example, in U.S. Pat. No. 5,252,479, and in WIPO publications WO 92/07573, WO 90/06997, WO 89/05345, WO 92/05266 and WO 92/14829, incorporated herein by reference, which provide a description of methods for efficiently introducing nucleic acids into human cells using such retroviral vectors. Other retroviral vectors include, for example, the mouse mammary tumor virus vectors (e.g., Shackleford et al., Proc. Natl. Acad. Sci. USA, 85:9655-9659 (1988)), and the like.

In particular, the specificity of viral vectors for particular cell types can be utilized to target predetermined cell types. Thus, the selection of a viral vector will depend, in part, on the cell type to be targeted. For example, if a neurodegenerative disease is to be treated by increasing the level of a CARD-containing polypeptide in neuronal cells affected by the disease, then a viral vector that targets neuronal cells can be used. A vector derived from a herpes simplex virus is an example of a viral vector that targets neuronal cells (Battleman et al., J. Neurosci. 13:941-951 (1993), which is incorporated herein by reference). Similarly, if a disease or pathological condition of the hematopoietic system is to be treated, then a viral vector that is specific for a particular blood cell or its precursor cell can be used. A vector based on a human immunodeficiency virus is an example of such a viral vector (Carroll et al., J. Cell. Biochem. 17E:241 (1993), which is incorporated herein by reference). In addition, a viral vector or other vector can be constructed to express a CARD-encoding nucleic acid in a tissue specific manner by incorporating a tissue-specific promoter or enhancer into the vector (Dai et al., Proc. Natl. Acad. Sci. USA 89:10892-10895 (1992), which is incorporated herein by reference).

For gene therapy, a vector containing a CARD-encoding nucleic acid or an antisense nucleotide sequence can be administered to a subject by various methods. For example, if viral vectors are used, administration can take advantage of the target specificity of the vectors. In such cases, there in no need to administer the vector locally at the diseased site. However, local administration can be a particularly effective method of administering a CARD-encoding nucleic acid. In addition, administration can be via intravenous or subcutaneous injection into the subject. Following injection, the viral vectors will circulate until they recognize host cells with the appropriate target specificity for infection. Injection of viral vectors into the spinal fluid also can be an effective mode of administration, for example, in treating a neurodegenerative disease.

Receptor-mediated DNA delivery approaches also can be used to deliver a CARD-encoding nucleic acid molecule into cells in a tissue-specific manner using a tissue-specific ligand or an antibody that is non-covalently complexed with the nucleic acid molecule via a bridging molecule (Curiel et al., Hum. Gene Ther. 3:147-154 (1992); Wu and Wu, J. Biol. Chem. 262:4429-4432 (1987), each of which is incorporated herein by reference). Direct injection of a naked or a nucleic acid molecule encapsulated, for example, in cationic liposomes also can be used for stable gene transfer into non-dividing or dividing cells in vivo (Ulmer et al., Science 259:1745-1748 (1993), which is incorporated herein by reference). In addition, a CARD-encoding nucleic acid molecule can be transferred into a variety of tissues using the particle bombardment method (Williams et al., Proc. Natl. Acad. Sci. USA 88:2726-2730 (1991), which is incorporated herein by reference). Such nucleic acid molecules can be linked to the appropriate nucleotide sequences required for transcription and translation.

A particularly useful mode of administration of a CARD-encoding nucleic acid is by direct inoculation locally at the site of the disease or pathological condition. Local administration can be advantageous because there is no dilution effect and, therefore, the likelihood that a majority of the targeted cells will be contacted with the nucleic acid molecule is increased. Thus, local inoculation can alleviate the targeting requirement necessary with other forms of administration and, if desired, a vector that infects all cell types in the inoculated area can be used. If expression is desired in only a specific subset of cells within the inoculated area, then a promoter, an enhancer or other expression element specific for the desired subset of cells can be linked to the nucleic acid molecule. Vectors containing such nucleic acid molecules and regulatory elements can be viral vectors, viral genomes, plasmids, phagemids and the like. Transfection vehicles such as liposomes also can be used to introduce a non-viral vector into recipient cells. Such vehicles are well known in the art.

The present invention also provides therapeutic compositions useful for practicing the therapeutic methods described herein. Therapeutic compositions of the present invention, such as pharmaceutical compositions, contain a physiologically compatible carrier together with an invention CARD-containing polypeptide (or functional fragment thereof), an invention CARD-encoding nucleic acid, an agent that alters CARD activity or expression identified by the methods described herein, or an anti-CARD antibody, as described herein, dissolved or dispersed therein as an active ingredient. In a preferred embodiment, the therapeutic composition is not immunogenic when administered to a mammal or human patient for therapeutic purposes.

As used herein, the terms “pharmaceutically acceptable”, “physiologically compatible” and grammatical variations thereof, as they refer to compositions, carriers, diluents and reagents, are used interchangeably and represent that the materials are capable of administration to a mammal without the production of undesirable physiological effects.

The preparation of a pharmacological composition that contains active ingredients dissolved or dispersed therein is well known in the art. Typically such compositions are prepared as injectibles either as liquid solutions or suspensions; however, solid forms suitable for solution, or suspension, in liquid prior to use can also be prepared. The preparation can also be emulsified.

The active ingredient can be mixed with excipients which are pharmaceutically acceptable and compatible with the active ingredient in amounts suitable for use in the therapeutic methods described herein. Suitable excipients are, for example, water, saline, dextrose, glycerol, ethanol, or the like, as well as combinations of any two or more thereof. In addition, if desired, the composition can contain minor amounts of auxiliary substances such as wetting or emulsifying agents, pH buffering agents, and the like, which enhance the effectiveness of the active ingredient.

The therapeutic composition of the present invention can include pharmaceutically acceptable salts of the components therein. Pharmaceutically acceptable nontoxic salts include the acid addition salts (formed with the free amino groups of the polypeptide) that are formed with inorganic acids such as, for example, hydrochloric acid, hydrobromic acid, perchloric acid, nitric acid, thiocyanic acid, sulfuric acid, phosphoric acid, acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid, and the like.

Salts formed with the free carboxyl groups can also be derived from inorganic bases such as, for example, sodium hydroxide, ammonium hydroxide, potassium hydroxide, and the like; and organic bases such as mono-, di-, and tri-alkyl and -aryl amines (e.g., triethylamine, diisopropyl amine, methyl amine, dimethyl amine, and the like) and optionally substituted ethanolamines (e.g., ethanolamine, diethanolamine, and the like).

Physiologically tolerable carriers are well known in the art. Exemplary liquid carriers are sterile aqueous solutions that contain no materials in addition to the active ingredients and water, or contain a buffer such as sodium phosphate at physiological pH, physiological saline or both, such as phosphate-buffered saline. Still further, aqueous carriers can contain more than one buffer salt, as well as salts such as sodium and potassium chlorides, dextrose, polyethylene glycol and other solutes.

Liquid compositions can also contain liquid phases in addition to and to the exclusion of water. Exemplary additional liquid phases include glycerin, vegetable oils such as cottonseed oil, and water-oil emulsions.

As described herein, an “effective amount” is a predetermined amount calculated to achieve the desired therapeutic effect, i.e., to alter the protein binding activity of a CARD-containing polypeptide or the catalytic activity of a CARD-containing polypeptide, resulting in altered biochemical process modulated by a CARD-containing polypeptide. The required dosage will vary with the particular treatment and with the duration of desired treatment; however, it is anticipated that dosages between about 10 micrograms and about 1 milligram per kilogram of body weight per day will be used for therapeutic treatment. It may be particularly advantageous to administer such agents in depot or long-lasting form as discussed herein. A therapeutically effective amount is typically an amount of an agent identified herein that, when administered in a physiologically acceptable composition, is sufficient to achieve a plasma concentration of from about 0.1 μg/ml to about 100 μg/ml, preferably from about 1.0 μg/ml to about 50 μg/ml, more preferably at least about 2 μg/ml and usually 5 to 10 μg/ml. Therapeutic invention anti-CARD antibodies can be administered in proportionately appropriate amounts in accordance with known practices in this art.

Also provided herein are methods of treating pathologies characterized by abnormal cell proliferation, abnormal cell death, or inflammation the method comprising administering an effective amount of an invention therapeutic composition. Such compositions are typically administered in a physiologically compatible composition.

Exemplary abnormal cell proliferation diseases associated with CARD-containing polypeptides contemplated herein for treatment according to the present invention include cancer pathologies, keratinocyte hyperplasia, neoplasia, keloid, benign prostatic hypertrophy, inflammatory hyperplasia, fibrosis, smooth muscle cell proliferation in arteries following balloon angioplasty (restenosis), and the like. Exemplary cancer pathologies contemplated herein for treatment include, gliomas, carcinomas, adenocarcinomas, sarcomas, melanomas, hamartomas, leukemias, lymphomas, and the like. Further diseases associated with CARD-containing polypeptides contemplated herein for treatment according to the present invention include inflammatory diseases and diseases of cell loss. Such diseases include allergies, inflammatory diseases including arthritis, lupus, Schrogen's syndrome, Crohn's disease, ulcerative colitis, as well as allograft rejection, such as graft-versus-host disease, and the like. CARD-containing polypeptides can also be useful in design of strategies for preventing diseases related to abnormal cell death in conditions such as stroke, myocardial infarction, heart failure, neurodegenerative diseases such as Parkinson's and Alzheimer's diseases, and for immunodeficiency associated diseases such as HIV infection, HIV-related disease, and the like.

Methods of treating pathologies can include methods of modulating the activity of one or more oncogenic proteins, wherein the oncogenic proteins specifically interact with a CARD-containing polypeptide of the invention. Methods of modulating the activity of such oncogenic proteins will include contacting the oncogenic protein with a substantially pure CARD-containing polypeptide or an active fragment (i.e., oncogenic protein-binding fragment) thereof. This contacting will alter the activity of the oncogenic protein, thereby providing a method of treating a pathology caused by the oncogenic protein. Further methods of modulating the activity of oncogenic proteins will include contacting the oncogenic protein with an agent, wherein the agent alters interaction between a CARD-containing polypeptide and an oncogenic protein.

Also contemplated herein, are therapeutic methods using invention pharmaceutical compositions for the treatment of pathological disorders in which there is too little cell division, such as, for example, bone marrow aplasias, immunodeficiencies due to a decreased number of lymphocytes, and the like. Methods of treating a variety of inflammatory diseases with invention therapeutic compositions are also contemplated herein, such as treatment of sepsis, fibrosis (e.g., scarring), arthritis, graft versus host disease, and the like.

The present invention also provides methods for diagnosing a pathology that is characterized by an increased or decreased level of a biochemical process to determine whether the increased or decreased level of the biochemical process is due, for example, to increased or decreased expression of a CARD-containing polypeptide or to expression of a variant CARD-containing polypeptide. As disclosed herein, such biochemical processes include apoptosis, NF-kB induction, cytokine processing, caspase-mediated proteolysis, transcription, inflammation, cell adhesion, and the like. The identification of such a pathology, which can be due to altered association of a CARD-containing polypeptide with a CARD-associated polypeptide in a cell, or altered ligand binding or catalytic activity of a CARD-containing polypeptide, can allow for intervention therapy using an effective agent or a nucleic acid molecule or an antisense nucleotide sequence as described herein. In general, a test sample can be obtained from a subject having a pathology characterized by having or suspected of having increased or decreased apoptosis and can be compared to a reference sample from a normal subject to determine whether a cell in the test sample has, for example, increased or decreased expression of a CARD-encoding gene. The level of a CARD-containing polypeptide in a cell can be determined by contacting a sample with a reagent such as an anti-CARD antibody or a CARD-associated polypeptide, either of which can specifically bind a CARD-containing polypeptide. For example, the level of a CARD-containing polypeptide in a cell can determined by well known immunoassay or immunohistochemical methods using an anti-CARD antibody (see, for example, Reed et al., Anal. Biochem. 205:70-76 (1992); see, also, Harlow and Lane, supra, (1988)). As used herein, the term “reagent” means a chemical or biological molecule that can specifically bind to a CARD-containing polypeptide or to a bound CARD/CARD-associated polypeptide complex. For example, either an anti-CARD antibody or a CARD-associated polypeptide can be a reagent for a CARD-containing polypeptide, whereas either an anti-CARD antibody or an anti-CARD-associated polypeptide antibody can be a reagent for a CARD/CARD-associated polypeptide complex.

As used herein, the term “test sample” means a cell or tissue specimen that is obtained from a subject and is to be examined for expression of a CARD-encoding gene in a cell in the sample. A test sample can be obtained, for example, during surgery or by needle biopsy and can be examined using the methods described herein to diagnose a pathology characterized by increased or decreased apoptosis. Increased or decreased expression of a CARD-encoding gene in a cell in a test sample can be determined, for example, by comparison to an expected normal level of CARD-containing polypeptide or CARD-encoding mRNA in a particular cell type. A normal range of CARD-containing polypeptide or CARD-encoding mRNA levels in various cell types can be determined by sampling a statistically significant number of normal subjects. In addition, a reference sample can be evaluated in parallel with a test sample in order to determine whether a pathology characterized by increased or decreased apoptosis is due to increased or decreased expression of a CARD-encoding gene. The test sample can be examined using, for example, immunohistochemical methods as described above or the sample can be further processed and examined. For example, an extract of a test sample can be prepared and examined to determine whether a CARD-containing polypeptide in the sample can associate with a CARD-associated polypeptide in the same manner as a CARD-containing polypeptide from a reference cell or whether, instead, a variant CARD-containing polypeptide is expressed in the cell.

In accordance with another embodiment of the present invention, there are provided diagnostic systems, preferably in kit form, comprising at least one invention CARD-encoding nucleic acid, CARD-containing polypeptide, and/or anti-CARD antibody described herein, in a suitable packaging material. In one embodiment, for example, the diagnostic nucleic acids are derived from any of SEQ ID NOS:11, 187, 96, 98, 100, 102, 85 and 89. Invention diagnostic systems are useful for assaying for the presence or absence of CARD-encoding nucleic acid in either genomic DNA or in transcribed CARD-encoding nucleic acid, such as mRNA or cDNA.

A suitable diagnostic system includes at least one invention CARD-encoding nucleic acid, CARD-containing polypeptide, and/or anti-CARD antibody, preferably two or more invention nucleic acids, proteins and/or antibodies, as a separately packaged chemical reagent(s) in an amount sufficient for at least one assay. Instructions for use of the packaged reagent are also typically included. Those of skill in the art can readily incorporate invention nucleic acid probes and/or primers into kit form in combination with appropriate buffers and solutions for the practice of the invention methods as described herein.

As employed herein, the phrase “packaging material” refers to one or more physical structures used to house the contents of the kit, such as invention nucleic acid probes or primers, and the like. The packaging material is constructed by well known methods, preferably to provide a sterile, contaminant-free environment. The packaging material has a label which indicates that the invention nucleic acids can be used for detecting a particular CARD-encoding sequence including the nucleotide sequences set forth in SEQ ID NOS:11, 187, 96, 98, 100, 102, 85 and 89 or mutations or deletions therein, thereby diagnosing the presence of, or a predisposition for a pathology such as cancer or an autoimmune disease. In addition, the packaging material contains instructions indicating how the materials within the kit are employed both to detect a particular sequence and diagnose the presence of, or a predisposition for a pathology such as cancer or an autoimmune disease.

The packaging materials employed herein in relation to diagnostic systems are those customarily utilized in nucleic acid-based diagnostic systems. As used herein, the term “package” refers to a solid matrix or material such as glass, plastic, paper, foil, and the like, capable of holding within fixed limits an isolated nucleic acid, oligonucleotide, or primer of the present invention. Thus, for example, a package can be a glass vial used to contain milligram quantities of a contemplated nucleic acid, oligonucleotide or primer, or it can be a microtiter plate well to which microgram quantities of a contemplated nucleic acid probe have been operatively affixed.

“Instructions for use” typically include a tangible expression describing the reagent concentration or at least one assay method parameter, such as the relative amounts of reagent and sample to be admixed, maintenance time periods for reagent/sample admixtures, temperature, buffer conditions, and the like.

A diagnostic assay should include a simple method for detecting the amount of a CARD-containing polypeptide or CARD-encoding nucleic acid in a sample that is bound to the reagent. Detection can be performed by labeling the reagent and detecting the presence of the label using well known methods (see, for example, Harlow and Lane, supra, 1988; chap. 9, for labeling an antibody). A reagent can be labeled with various detectable moieties including a radiolabel, an enzyme, biotin or a fluorochrome. Materials for labeling the reagent can be included in the diagnostic kit or can be purchased separately from a commercial source. Following contact of a labeled reagent with a test sample and, if desired, a control sample, specifically bound reagent can be identified by detecting the particular moiety.

A labeled antibody that can specifically bind the reagent also can be used to identify specific binding of an unlabeled reagent. For example, if the reagent is an anti-CARD antibody, a second antibody can be used to detect specific binding of the anti-CARD antibody. A second antibody generally will be specific for the particular class of the first antibody. For example, if an anti-CARD antibody is of the IgG class, a second antibody will be an anti-IgG antibody. Such second antibodies are readily available from commercial sources. The second antibody can be labeled using a detectable moiety as described above. When a sample is labeled using a second antibody, the sample is first contacted with a first antibody, then the sample is contacted with the labeled second antibody, which specifically binds to the first antibody and results in a labeled sample.

In accordance with another embodiment of the invention, there are provided methods for determining a prognosis of disease free or overall survival in a patient suffering from cancer. For example, it is contemplated herein that abnormal levels of CARD-containing polypeptides (either higher or lower) in primary tumor tissue show a high correlation with either increased or decreased tumor recurrence or spread, and therefore indicates the likelihood of disease free or overall survival. Thus, the present invention advantageously provides a significant advancement in cancer management because early identification of patients at risk for tumor recurrence or spread will permit aggressive early treatment with significantly enhanced potential for survival. Also provided are methods for predicting the risk of tumor recurrence or spread in an individual having a cancer tumor; methods for screening a cancer patient to determine the risk of tumor metastasis; and methods for determining the proper course of treatment for a patient suffering from cancer. These methods are carried out by collecting a sample from a patient and comparing the level of CARD-encoding gene expression in the patient to the level of expression in a control or to a reference level of CARD-encoding gene expression as defined by patient population sampling, tissue culture analysis, or any other method known for determining reference levels for determination of disease prognosis. The level of CARD-encoding gene expression in the patient is then classified as higher than the reference level or lower than the reference level, wherein the prognosis of survival or tumor recurrence is different for patients with higher levels than the prognosis for patients with lower levels.

All U.S. patents and all publications mentioned herein are incorporated in their entirety by reference thereto. The invention will now be described in greater detail by reference to the following non-limiting examples.

### EXAMPLES

1.0 Identification of CARD-Containing Polypeptides.

The process of gene identification and assembling include the following steps:

A) Identification of new candidate CARD containing polypeptides. A database search was performed using the TBLASTN program with the CARD domain of caspase-1 and caspase-12 as the query in the following NCBI databases: high throughput genome sequence (HTGS), genomic survey sequence (GSS) and expressed sequence tag (EST) databases.

B) Verification that the new candidate CARD containing polypeptide is novel. Using PSI-BLAST, each new candidate CARD gene was queried in the annotated non-redundant (NR) database at NCBI. When the new candidate gene showed significant but not identical homology with other known CARD containing polypeptides during this search, the CARD containing polypeptide candidate was kept for further analysis.

C) 3-D-Model Building of new candidate CARD polypeptide: When the sequence homology was low (<25% identity), three-dimensional criteria was added to characterization of new CARD-containing polypeptides. The candidate CARD fragment was analyzed by a profile-profile sequence comparison method which aligns the candidate CARD domain with a database of sequences of known three-dimensional structure. From this analysis, a sequence alignment was produced and a three-dimensional model was built according to the known structure of CARD domain of IAP-1. In most cases, the best score was produced using CARD domain sequences having known three-dimensional structures. The quality of the three-dimensional model obtained from the alignments confirmed that novel CARD-domain containing polypeptides had been identified.

D) Identification of additional domains in the full length protein. Full length protein sequences were obtained using the closest full-length caspase homolog of the new CARD identified in step B as query. TBLASTN searches of the sequences containing the newly identified CARD domains were performed. Longer aligned fragments or multiple aligned fragments in the accession number corresponding to the newly identified CARD containing polypeptides indicated a longer protein.

E) These additional domains were assembled using the following gene building procedure:

Genomic DNA fragments were identified by T-BLAST-N analysis using mouse caspase-12 and human caspase-1 full length protein as query and scanning HTGS database from NCBI of incomplete DNA genomics sequences. New fragments homologous to caspase-12 and caspase-1 were further confirmed by psi-blast analysis using the TBLASTN genomic DNA homolog fragment as query and scanning NR database. The boundary of each fragment was identified according to the following criteria:

Disruption of sequence similarity between the protein alignment of the target fragment and the query.

Extension of the protein sequence alignment between query and target using ORF finder.

Protein sequence overlap between two contiguous fragments in sequence relative to the query.

Conservation of exon-intron junction between DNA sequence of the target and query.

Orientation of the ORF of the different genomic DNA fragment.

Presence of contiguous fragments, based on sequence alignment with the query, on the same contig.

Finally, the reconstituted sequences were aligned by CLUSTALW with the query and exon-intron junctions further refined by repeating the above process.

2.0 Identification of CARD2X, CARD3X and CLAN. Nucleic acids encoding CARD containing proteins CARD2X, CARD3X and CLAN were identified from different CARD queries using tblastn and systematically scanning gss, htgs, and all EST databases at NCBI. Further analysis using translated genomic fragment containing CARD domains larger than the CARD domain itself as query were performed to identify additional domains. Genomic DNA were translated in all reading frames and examined for additional domains using psi-blast and nr database.

3.0 Cloning and sequencing of large cDNA. For cDNA larger than 1500 bp, cloning is accomplished by amplification of multiple fragments of the cDNA. Jurkat total RNA is reverse-transcribed to complementary DNAs using MMLV reverse transcriptase (Stratagene) and random hexanucleotide primers. Overlapping cDNA fragments of a CARD-containing polypeptide are amplified from the Jurkat complementary DNAs with Turbo Pfu DNA polymerase (Stratagene) using an oligonucleotide primer set for every 1500 bp of cDNA, where the amplified cDNA fragment contains a unique restriction site near the end that is to be ligated with an adjacent amplified cDNA fragment.

The resultant cDNA fragments are ligated into mammalian expression vector pcDNA-myc (Invitrogen, modified as described in Roy et al., EMBO J. 16:6914-6925 (1997)) and assembled to full-length cDNA by consecutively ligating adjacent fragments at the unique endonuclease sites form the full-length cDNA. Sequencing analysis of the assembled full-length cDNA is carried out, and splice isoforms of CARD-containing polypeptides can be identified.

4.0 Plasmid Constructions. Complementary DNA encoding a CARD-containing polypeptide, or a functional fragment thereof is amplified from Jurkat cDNAs with Turbo Pfu DNA polymerase (Stratagene) and desired primers, such as those described above. The resultant PCR fragments are digested with restriction enzymes such as EcoRI and Xho I and ligated into pGEX-4T1 (Pharmacia) and pcDNA-myc vectors.

5.0 In vitro Protein Binding Assays. CARD-containing or fragments thereof encoded in pGEX-4T1 are expressed in XL-1 blue E. coli cells (Stratagene), and affinity-purified using glutathione (GSH)-sepharose according to known methods, such as those in Current Protocols in Molecular Biology, Ausubel et al. eds., John Wiley and Sons (1999). For GST pull-down assays, purified CARD-GST fusion proteins and GST alone (0.1-0.5 μg immobilized on 10-15 μl GSH-sepharose beads) are incubated with 1 mg/ml of BSA in 100 μl Co-IP buffer (142.4 mM KCl, 5 mM MgCl₂, 10 mM HEPES (pH 7.4), 0.5 mM EGTA, 0.2% NP-40, 1 mM DTT, and 1 mM PMSF) for 30 min. at room temperature. The beads are then incubated with 1 μl of rat reticulocyte lysates (TnT-lysate; Promega, Inc.) containing ³⁵S-labeled, in vitro translated CARD-containing or control protein Skp-1 in 100 μl Co-IP buffer supplemented with 0.5 mg/ml BSA for overnight at 4° C. The beads are washed four times in 500 μl Co-IP buffer, followed by boiling in 20 μl Laemmli-SDS sample buffer. The eluted proteins are analyzed by SDS-PAGE. The bands of SDS-PAGE gels are detected by fluorography.

The resultant oligomerization pattern will reveal that CARD:CARD and other protein:protein interactions occur with CARD-containing polypeptides or fragments thereof.

In vitro translated candidate CARD-associated polypeptides such as Apaf-1 (lacking its WD domain), CED4, and control Skp-1 are subjected to GST pull-down assay using GSH-sepharose beads conjugated with GST and GST-CARD-containing polypeptides as described above. Lanes containing GST-CARD yield significant signals when incubated with a CARD-associated polypeptide whereas, the controls GST alone and Skp-1 yield negligible signals.

6.0 Protein Interaction Studies in Yeast. EGY48 yeast cells (Saccharomyces cerevisiae: MATα, trpl, ura3, his, leu2::plexApo6-leu2) are transformed with pGilda-CARD plasmids (his marker) encoding the LexA DNA binding domain fused to: CARD-containing polypeptides, fragments thereof, or CARD-associated polypeptides. EGY48 are also transformed with a LexA-LacZ reporter plasmid pSH1840 (ura3 marker), as previously described (Durfee et al., 1993; Sato et al., 1995). Sources for cells and plasmids are described previously in U.S. Pat. No. 5,632,994, and in Zervous et al., Cell 72:223-232 (1993); Gyuris et al., Cell 75:791-803 (1993); Golemis et al., In Current Protocols in Molecular Biology (ed. Ausubel et al.; Green Publ.; NY 1994), each of which is incorporated herein by reference. Transformants are replica-plated on Burkholder's minimal medium (BMM) plates supplemented with leucine and 2% glucose as previously described (Sato et al., Gene 140:291-292 (1994)). Protein-protein interactions are scored by growth of transformants on leucine deficient BMM plates containing 2% galactose and 1% raffinose.

Protein-protein interactions are also evaluated using β-galactosidase activity assays. Colonies grown on BMM/Leu/Glucose plates are filter-lifted onto nitrocellulose membranes, and incubated over-night on BMM/Leu/galactose plates. Yeast cells are lysed by soaking filters in liquid nitrogen and thawing at room temperature. β-galactosidase activity is measured by incubating the filter in 3.2 ml Z buffer (60 mM, Na₂HPO₄, 40 mM Na₂HPO₄, 10 mM KCl, 1 mM MgSO₄) supplemented with 50 μl X-gal solution (20 mg/ml). Levels of β-galactosidase activity are scaled according to the intensity of blue color generated for each transformant.

The results of this experiment will show colonies on leucine deficient plates for yeast containing CARD/LexA fusions together with CARD-associated polypeptide/B42. In addition, the CARD/LexA:CARD-associated polypeptide/B42 cells will have significant amounts of LacZ activity.

7.0 Self-Association of NACHT domain of CARD-containing polypeptides. In vitro translated, ³⁵S-labeled rat reticulocyte lysates (1 μl) containing NACHT or Skp-1 (used as a control) are incubated with GSH-sepharose beads conjugated with purified GST-NACHT or GST alone for GST pull-down assay, resolved on SDS-PAGE and visualized by fluorography as described above. One tenth of input is loaded for NACHT or Skp-1 as controls.

8.0 Protein-Protein Interactions of CARD-containing polypeptides. Transient transfection of 293T, a human embryonic kidney fibroblast cell line, are conducted using SuperFect reagents (Qiagen) according to manufacturer's instructions. The cDNA fragments encoding full-length CED4 and the truncated form of Apaf-1 (Apaf-1ΔWD) comprising amino acids 1-420 of the human Apaf-1 protein are amplified by PCR and subcloned into pcDNA3HA at EcoRI and Xho I sites. Expression plasmids encoding catalytically inactive forms of caspases such as pro-Casp8 (pro-Casp8 (C/A)) are prepared by replacing Cys 377 with an Ala using site-directed mutagenesis and pro-Casp9 (pro-Casp9 (C/A)) has been described previously, Cardone et al., Science 282:1318-1321 (1998)). 293T cells are transiently transfected with an expression plasmid (2 μg) encoding HA-tagged human Apaf-1ΔWD, CED4, pro-Casp8 (C/A) or C-Terminal Flag-tagged pro-Casp9 (C/A) in the presence or absence of a plasmid (2 μg) encoding myc-tagged CARD-containing polypeptide. After 24 hours growth in culture, transfected cells are collected and lysed in Co-IP buffer (142.4 mM KCl, 5 mM MgCl₂, 10 mM HEPES (pH 7.4), 0.5 mM EGTA, 0.1% NP-40, and 1 mM DTT) supplemented with 12.5 mM β-glycerolphosphate, 2 mM NaF, 1 mM Na₃VO₄, 1 mM PMSF, and 1× protenase inhibitor mix (Boehringer Mannheim). Cell lysates are clarified by microcentrifugation and subjected to immunoprecipitation using either a mouse monoclonal antibody to myc (Santa Cruz Biotechnologies, Inc) or a control mouse IgG. Proteins from the immune complexes are resolved by SDS-PAGE, transferred to nitrocellulose membranes, and subjected to immunoblot analysis using anti-HA antibodies followed by anti-myc antibodies using a standard Western blotting procedure and ECL reagents from Amersham-Pharmacia Biotechnologies, Inc. (Krajewski et al., Proc. Natl. Acad. Sci. USA 96:5752-5757 (1999)).

9.0 Cloning and characterization of CARD2X. CARD2X-encoding cDNA was obtained by PCR using primers CGGAATTCATGGCTACCGAGAGTACTCC (SEQ ID NO:76) and GTAAAACGACGGCCAGT (SEQ ID NO:77) to amplify a 0.9 kb cDNA molecule from a human skeletal muscle cDNA library (Clontech). The PCR products was then purified by agarose gel electrophoresis and the purified products subcloned into pBluescript II SK vector (Stratagene). Using the forward primers, the PCR fragments were directly sequenced using the ABI PRISM Big Dye Terminal Cycle sequencing kit, according to manufacturer's instructions (Perkin Elmer). Based on the sequence obtained, a third CARD2X-specific primer was generated having the sequence GCAGAAGCCACTGTGGAAGAGGAGGTT (SEQ ID NO:78). In identifying the 3′ end of the CARD2X-encoding cDNA, this third CARD2X-specific primer was used in conjunction with a phage-specific primer having the sequence ATACGACTCACTATAGGGCGAATTGGCC (SEQ ID NO:79) to amplify a 0.3 kb cDNA molecule using methods described above. The 0.3 kb cDNA molecule was cloned and sequenced as described above, and the sequences of the 0.3 and 0.9 kb cDNA molecules were merged to produce a 1.0 kb cDNA sequence.

The sequence of CARD2X was confirmed. Additional 5′ untranslated sequence was identified (nucleotide sequence of CARD2X including 5′ untranslated sequence, SEQ ID NO:84). The CARD domain extends from amino acids 4 to 78 of SEQ ID NO:12.

The association between CARD2X and other CARD-containing proteins was determined. HEK 293T cells in 6-well plates were transfected using SuperFect (Qiagen) with pairwise combinations of Myc-tagged or FLAG-tagged CARD2X, CARDIAK or NOD1 (total DNA 2 μg). After 24 hours, cells were collected in 400 μl of lysis buffer (20 mM Tris, pH 7.4, 150 mM NaCl, 1% NP-40, and 1 mM EDTA supplemented with 1× protease inhibitors mix (Roche/Boehringer Mannheim)). Cell lysates were clarified by centrifugation and subjected to immunoprecipitation using Agarose-beads conjugated with anti-FLAG M2 antibody (Sigma). Immune-complexes were washed three times with wash buffer (20 mM Tris, pH 7.4, 100 mM NaCl, 0.05% NP-40, and 1 mM EDTA), and resolved on SDS-PAGE gels. Proteins in the gels were transferred to nitrocellulose membranes, immunoblotted with anti-Myc antibodies, and detected with ECL (Amersham-Pharmacia Biotech). Epitope-specific antibodies for myc, FLAG, or HA tag were obtained from Santa Cruz Biotech, Roche/Boehringer Mannheim, and Sigma. The results of these co-immunoprecipitation assays demonstrated that CARD2X specifically associates with both NOD1 and with CARDIAK.

The effect of CARDIAK on CARD2X phosphorylation was next determined. HEK 293T cells transiently expressing FLAG-CARDIAK were lysed and immunoprecipitated with Agarose-beads conjugated with anti-FLAG M2 antibody. In vitro phosphorylation was performed in the immune complex with or without purified Myc-CARD-2× as a substrate. The kinase reaction was initiated by adding 1 μM of [γ-³²P]ATP in 10 μl of kinase buffer (50 mM Tris, pH7.4, 100 mM NaCl, 6 mM MgCl₂, 1 mM MnCl, and 1 mM EDTA). After 20 min at 37° C., the reaction was stopped by adding 10 μl of 2×SDS sample buffer, and subjected to SDS-PAGE and autoradiography. The results of these assays indicated that CARD2X is not phosphorylated directly by CARDIAK.

Phosphatase assays were also performed to examine phosphorylation of CARD2X. HEK 293 cells were transfected with plasmids encoding Myc-CARD-2× with or without FLAG-CARDIAK or FLAG-CARDIAK(K47M), which is a kinase deficient mutant of CARDIAK. The cleared lysates were diluted 1:20 with 20 μl of reaction buffer (25 mM Tris, pH8.0, 50 mM NaCl, 5 mM MgCl₂), and optionally treated with 2 units of calf intestine alkaline phosphatase (Gibco BRL) for 30 min at 37° C. The reaction was terminated by adding 7 μl 4× SDS sample buffer, and subjected to SDS-PAGE and immunoblot. The phosphorylated form of CARD2X migrates more slowly that CARD2X, and is not observed after phosphatase treatment. The results of these assays indicated that CARD2X is phosphorylated in vivo in the presence of either CARDIAK or kinase-deficient CARDIAK, but not in their absence. Taken together with the in vitro phosphorylation results above, these results indicate that CARDIAK is indirectly involved in CARD2X phosphorylation.

The 30-35 residues at the carboxy terminus of CARD2X have homology to human Alu family sequences and RhoGAP. Thus, this region can have activity similar to that observed in human Alu family sequences and RhoGAP.

10.0 Cloning and characterization of CLAN. CLAN encoding cDNA was obtained by polymerase chain reaction (PCR) using primers CXF1:TACTTACTTTGTCCCTTCA (SEQ ID NO:74) and CXR2:TATTTGTCCCCATCTCGTC (SEQ ID NO:75) to amplify cDNA from a human genomic library. Thirty cycles of PCR were carried out using Turbo Pfu DNA polymerase (Stratagene) at annealing temperature 47° C. and extension temperature 72° C. The PCR product was then purified by agarose gel electrophoresis and the purified product subcloned into pGEM-T vector (Promega).

The HTSG database of human genomic DNA sequence data was searched for regions capable of encoding CARDs using the CARD amino-acid sequence of cIAP-1 as a query with the TBLASTn method. This search revealed strong homology with a human genomic clone (Accession number: AQ889169) that mapped to human chromosome 2p21-22. This locus was not recognized in the human genomic database and was not previously annotated. In initial studies, two genes encoding CARD domain containing polypeptides, designated CARD4X and CARD5X, were identified. Upon further characterization, it was determined that CARD4X (also known as NAC-X or NAC-4) and CARD5X were actually encoded by the same gene, which is therefore referenced as CARD4/5X. CARD4/5X was subsequently designated CLAN, which stands for “CARD, LRR and NACHT-containing protein,” because at least one of the proteins encoded by it contains CARD, Leucine Rich Repeat (LRR) and NACHT domains, as described below.

The CLAN gene locus lies in close proximity to the gene encoding Spastin (on chromosome 2p21-22), a AAA protein which is frequently mutated in autosomal dominant hereditary spastic paraplegia (AD-HSP). The CLAN locus is found on the strand opposite the SPG4 (SPAST) locus but with no overlapping regions. This result suggests that mutations in the CLAN gene potentially occur in patients with this neurodegenerative disorder.

Using GENESCAN for exon prediction, additional regions potentially encoding a NACHT domain and regions corresponding to Leucine-Rich Repeat (LRR) domains were also recognized 3′ to the potential CARD-encoding sequences, suggesting the presence of a CED4-like gene.

10.1 Cloning of CLAN cDNAs. CLAN-specific primers corresponding to sequences within the putative CARD and NACHT regions (as determined from genomic DNA sequence data) were used in conjunction with 2 universal primers to isolate CLAN cDNAs from first-strand liver and lung cDNA by nested PCR according to the manufacturer's protocol (SMART RACE, Clontech). Primers used for amplification are 5′ RACE primers (5′-CATGTGAATGATCCCTCTAGCAG-3′ (SEQ ID NO:153); nested 5′-GGGCTCGGCTATCGTGCTCTA-3′ (SEQ ID NO:154)) and 3′ RACE primers (5′-ACGATAGCCGAGCCCTTATTC-3′ (SEQ ID NO:155); nested 5′-GTATGGAATGTTCTGAATCGC-3′ (SEQ ID NO;156)). Amplification products were purified from agarose gels, ligated into the TA cloning vector (Promega), and sequenced. Four open reading frames were deduced and multiple clones of each isoform were sequenced to ensure fidelity of PCR products.

The longest transcript, termed CLAN-A, was 3.370 kilobasepairs (kbp) in length (SEQ ID NO:96) with an open reading frame (ORF) coding for a 1024 amino-acid protein (SEQ ID NO:97) containing a CARD, NACHT, and LRR domains, as well as a predicted SAM domain. A second transcript, termed CLAN-B, was 1.374 kbp in length (SEQ ID NO:98), with an ORF coding for a 359 amino-acid protein (SEQ ID NO:99) containing an identical CARD directly spliced to the LRRs. CLAN-C, the third transcript isolated, was 0.768 kbp in length (SEQ ID NO:102) and encoded a 156 amino acid protein (SEQ ID NO:103) containing the CARD and an additional region lacking homology to recognizable domains. Finally, the shortest transcript found, CLAN-D, was 0.578 kbp in length (SEQ ID NO:100) and contained an ORF encoding a 92 amino-acid protein (SEQ ID NO:101) encompassing only the CARD followed by 9 amino acids.

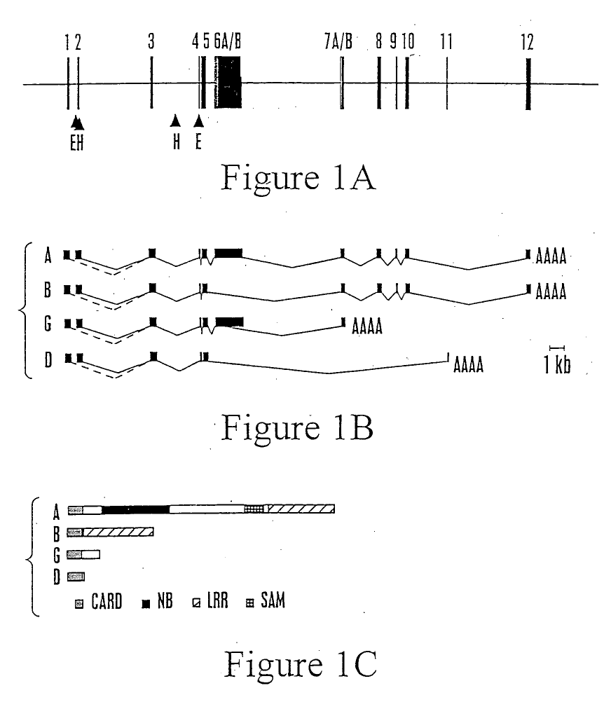

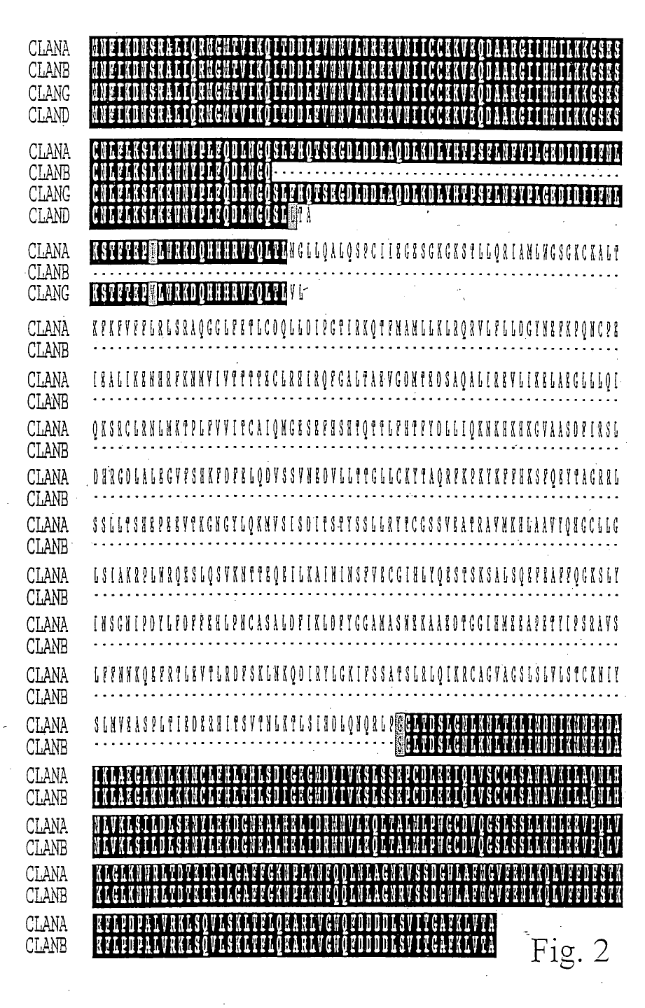

Comparisons of these cDNA sequence data with the genomic DNA sequence data found in the HTSG database suggested that the CLAN gene consists of 12 exons, spanning 41.3 kbp on chromosome 2p21-22 (FIG. 1A). Six differences were found between the sequence of the CLAN cDNA and the sequence within the public database. Additionally, nucleotide regions 1-12 and 3372-3396 do not have equivalent fragments in the public database.

Southern blot analysis was also performed. For Southern blot analysis, 10 μg of restriction endonuclease (EcoRI or PstI) digested genomic DNA was loaded per lane and hybridized with the CARD domain of CLAN as a probe. The probe was derived from the CLAN A-isoform (see FIGS. 1 and 2), nucleotides 276 to 507 plus an additional 20 upstream nucleotides, which are not present in the cDNA but are present in the genomic DNA. CLAN was found to be a single copy gene.

Two different transcriptional start sites are utilized (corresponding to the beginning of either exon 1 or 2); however both are spliced to exon 3 at the beginning of the CARD. Exons 6 and 7 contain additional internal splice donor sites which are utilized to generate CLAN-G. FIG. 1B shows the pattern of mRNA splicing events predicted to give rise to the CLAN-A, CLAN-B, CLAN-C, and CLAN-D transcripts and encoded proteins. All the exon/intron splice junctions follow the conserved GT/AG consensus rule.

As predicted by SMART (EMBL, Heidelberg, Germany), CLAN contains a CARD (amino acids 1-87 of SEQ ID NO:97). A ψ-BLAST search of the non-redundant database using the CLAN CARD as query identified several homologous CARDs including those from cIAP1 and 2 (58%), caspase-1 and ICEBERG (50%), Nod1, Nod2, and Card8 (˜38%) and caspase-13, Ced3, caspase-9, Bcl10 (CIPER) and CARKIAK/RIP2 (˜30%).

Following the CARD, a domain containing consensus sequences for Walker A and B boxes is present (Walker et al., EMBO J. 8:945-951 (1982)) as well as additional characteristics of the family of NTPases termed the NACHT family (Koonin et al., Trends. Biochem. Sci. 25:2230224 (2000)). By ψ-BLAST search the NACHT domain of CLAN (“NB” in FIG. 1, amino acids 161-457 of SEQ ID NO:97) shows highest similarity to the NACHT domain of NAIP (60%), followed by Nod1 (49%) and Nod2 (47%).

Leucine Rich Repeat (LRR) domains are also found near the C-terminus of the A and B forms of the protein. The C-terminal end consists of four repeated LRRs, each containing a predicted β sheet and a helical structure, which is in agreement with the prototypical horseshoe-shaped structure of LRRs (Kobe et al., Curr. Opin. Struct. Biol. 5:409-416 (1999). LRR 1 (amino acids 760-791 of SEQ ID NO:97) represents a non-Kobe and Deisenhofer (non-K/D) LRR, whereas LRRs 2, 3, and 4 (amino acids 817-848; 845-876; and 934-965 of SEQ ID NO:97, respectively) are in accordance with Kobe and Deisenhofer (K/D) LRR. LRR 2 also shares sequence homology to a prototypical Ribonuclease Inhibitor type A (RI type A). By ψ-BLAST searches the LRRs show 49% sequence identity to the placental ribonuclease/angiogenin inhibitor (RAI).

Sequences located between the NACHT and LRR domains show some similarity to the sterile alpha motif (SAM) (amino acids 642-696 of SEQ ID NO:97), a domain built of five alpha helices originally found in proteins involved in numerous developmental processes. The SAM domain has been shown to function as a protein-protein interaction domain, with ability to homo-as well as hetero-oligomerize with other SAMs (Stapleton et al., Nat. Struct. Biol. 6:44-49 (1999)).

10.2 In vivo expression of CLAN. In order to determine which of the various splice variants of CLAN are expressed in adult human tissues, Northern blot analysis was performed. Hybridization probes corresponding to the common CARD domain of all 4 CLAN isoforms or the NACHT and LRR regions were radiolabeled by random priming with hexanucleotides (Roche) and α-32P-dCTP, or Digoxigenin-labeled with a commercially available kit (Roche), incubated with blots containing human poly(A)+ RNA derived from various human tissues (Origene), washed at high stringency, and exposed to X-ray film. Positive signals were detected by autoradiography or by immunoblotting with HRP-conjugated anti-DIG antibody and an enhanced chemiluminescence method (ECL) (Amersham).

Northern blot analysis with CARD of CLAN revealed expression of an approximately 1.5 kbp transcript corresponding to CLAN-B in nearly all tissues examined, with highest expression in lung and spleen. Northern blot analysis using the NACHT and LRR of CLAN-A as a probe revealed expression of an approximately 3.5 kbp mRNA corresponding to CLAN-A primarily in the lung.

To further explore the tissue-specific patterns of expression of CLAN splicing variants, RT-PCR assays were devised specific for the A, B, C, and D isoforms. A panel of cDNA specimens derived from various human tissues was utilized (Clontech), as well as blood cells, prepared as followed. Peripheral blood leukocytes were obtained from heparinized venous blood by Ficoll-Paque (Amersham) density-gradient centrifugation. Red blood cells were removed from granulocytes by short incubation in hypotonic lysis buffer. Monocytes were separated from lymphocytes by adherence to plastic dishes. Total RNA was isolated from cells using TRIZOL reagent (BRL) and 2 μg was used to generate cDNA in a reverse transcription reaction with Superscript II (BRL).

PCR was carried out on the cDNA samples in an Eppendorf thermal cycler using Taq polymerase (BRL) and the following isoform-specific primer pairs:

| | | |
|---|---|---|
| CLAN-A | 5′-GGTGGAGCAGGATGCTGCTAGAGG-3′, | (SEQ ID |
| | | NO: 159) |
| | 5′-CACAGTGGTCCAGGCTCCGAATGAAGTCA-3′; | (SEQ ID |
| | | NO: 160) |
| CLAN-B | 5′-CATCATTTGCTGCGAGAAGGTGGAG-3′, | (SEQ ID |
| | | NO: 161) |
| | 5′-TTAACTTGGATAACACTTGGCTAAG-3′; | (SEQ ID |
| | | NO: 162) |
| CLAN-C | 5′-GTAAACATCATTTGCTGCGAGAA-3′, | (SEQ ID |
| | | NO: 163) |
| | 5′-CCCGGGCAGGTAGAAGATGCTAT-3′; | (SEQ ID |
| | | NO: 164) |
| CLAN-D | 5′-AATTTCATAAAGGACAATAGCCGAG-3′, | (SEQ ID |
| | | NO: 165) |
| | 5′-TGTCTACTGTACTTTCTAAGCTGTT-3′. | (SEQ ID |
| | | NO: 166) |

RT-PCR analysis showed that CLAN-B was present throughout human tissues (brain, heart, kidney, liver, lung, pancreas, placenta, skeletal muscle, colon, ovary, leukocytes, prostate, small intestine, spleen, testis, thymus), consistent with the Northern blot analysis. In contrast, CLAN-A was restricted to lung, colon, brain, prostate, spleen and leukocytes, but not other tissues. Further analysis of leukocyte sub-populations revealed expression of the CLAN-A isoform predominantly in the monocyte cell fraction, with lower expression found in granulocytes and no expression in lymphocytes. Expression of CLAN-C was absent in all normal tissues tested, however, expression was evident in the cell line HEK293T, suggesting this transcript can be produced under some circumstances. CLAN-D transcripts were detected only in brain by RT-PCR.

RT-PCR was also performed on cell lines. RT-PCR was performed using the same CLAN primers as used for RT-PCR in normal tissues, as described above. RT-PCR was performed in various tumor derived cell lines: M2, OVCAR3, HEY, HaCaT, 293T, SKOV-3, Jurkat, BG-1, 697, HL-60, PC3, DU145, MDA-MB-231, MCF-7, MDA-MB-4, HS578T, BT-549, and T-47D. Beta-actin primers were used as a control. In contrast to normal tissue, the transcript for CLAN was mostly absent in the cell lines tested. Weak expression was found in the cell lines 697, MDA-MB-231, MVF-7, MDA-MB-4, HS578T, and T-47D.

10.3 CLAN protein interactions. Interactions between the CARD of CLAN and known CARD domains were tested in vitro and in vivo.

To test CLAN interactions with other molecules, an in vitro binding assay was performed. CLAN was in vitro translated in the absence of label (i.e., cold). Other cellular proteins were labeled in vitro with ³⁵S-Met: CLAN, caspase1, caspase2, caspase8, caspase9, caspase10, Apaf1, Apaf1-CARD, NACa, NAC-CARD, Bcl10, ASC, cIAP1, cIAP2, XIAP, Nod1, Ced4, RAIDD, and CARDIAK. The in vitro translated proteins were mixed separately with unlabeled CLAN and co-immunoprecipitated using an antibody against an epitope tag fused to CARD5X, either myc or hemaglutinin (HA). CLAN associated proteins were eluted by boiling in Laemmli denaturing buffer and separated by 12% SDS-PAGE. The radioactive bands were visualized by fluorography.

Weak binding to CLAN was observed with caspase2 and cIAP1, with stronger binding to Nod1 and Cardiak. The strongest binding was observed with Ced4. Caspase8 binding is possibly due to its stickiness. There was no association detected between CLAN and itself.

To prepare appropriate expression vectors for in vivo interaction studies, a cDNA encoding the CLAN CARD domain was amplified using PFU polymerase and specific primers (5′-CCCGGATCCATGAATTTCATAAAGGACAATAGC-3′ (SEQ ID NO:153); 5′-CCCTTCGAACAAGTCCTGAAATAGAGGATA-3′ (SEQ ID NO:154)) containing BamHI and HindIII sites. The resulting PCR product was ligated into pcDNA3.1 (−)/Myc-His6 A (Invitrogen) which places the myc-His₆ tag at the C-terminus of expressed proteins. pcDNA3/HA-CLAN (CARD) was created using a similar strategy. Authenticity of all vectors was confirmed by DNA sequencing.

The CARD of CLAN was expressed as an epitope-tagged protein in HEK293T cells in co-transfections with a variety of other epitope-tagged CARD-containing proteins, and lysates derived from these cells were used for co-immunoprecipitation assays. Briefly, HEK293T cells were seeded onto six-well plates (35 mm wells) and transfected with 0.2-2 mg plasmid DNA using Superfect (Qiagen) 24 hours later. After culturing for a day, cells were collected and lysed in isotonic lysis buffer (142.4 mM KCl, 5 mM MgCl₂, 10 mM HEPES (pH 7.4), 0.5 mM EGTA, 0.2% NP-40, 12.5 mM b-glycerophosphate, 2 mM NaF, 1 mM Na₃VO₄, 1 mM PMSF, and 1× protease inhibitor mix (Roche)). Lysates were clarified by centrifugation and subjected to immunoprecipitation using agarose-conjugated anti-c-myc antibodies (Santa Cruz), or non-specific control antibodies and Protein G-agarose for 2-24 hr at 4° C. Immune-complexes were washed four times with lysis buffer, boiled in Laemmli buffer, and separated by 12-15% PAGE. Immune-complexes were then transferred to PVDF membranes and immunoblotted with anti-c-myc (Santa Cruz), anti-HA (Roche), or anti-flag (Sigma) antibodies. Membranes were washed, incubated with HRP-conjugated secondary antibodies, and reactive proteins were detected using ECL.

Co-immunoprecipitation analysis indicated that the CARD of CLAN bound readily to full-length pro-caspase-1 but did not significantly bind another CARD-containing caspase, caspase-9. Among the other CED-4 family members which contain a CARD in conjunction with a nucleotide-binding domain, CLAN interacted with the CARDs of Nod2 and NAC, but not with Apaf-1 or Nod-1. Finally, the CLAN CARD was found to associate with Bcl-10, but not with another adapter protein, RAIDD.

11.0 Cloning and characterization of CARD3X Based on an analysis of the overlapping genomic contigs GI 8575872 and GI 5001450, a cDNA sequence for CARD3X was predicted (SEQ ID NO:82), that encoded amino acid sequences designated SEQ ID NOS:83 and 107.

For identification of novel domains in CARD3X, the sequence of the CARD domain of polypeptide CARD3X was used as a query for a tblastn search in the HTGS database, and two overlapping genomic contigs were found (GI numbers 5001450 and 8575872). This contig was analyzed using the GenScan server (http://ccr-081.mit.edu/GENSCAN.html) for the presence of exons. (Burge and Karlin, J. Mol. Biol. 268:78-94 (1997)). The predicted protein sequences coded by the exons were analyzed by comparison with the NCBI nr protein sequence database using PSI-BLAST. The predicted protein sequences coded by the exons were analyzed also by comparison with a database of proteins with known three-dimensional structures and apoptosis related domains using the profile-profile comparison server at http://bioinformatics.burnham-inst.org/FFAS_apoptosis (Rychlewski, et al., Protein Science 9:232-241 (2000)).

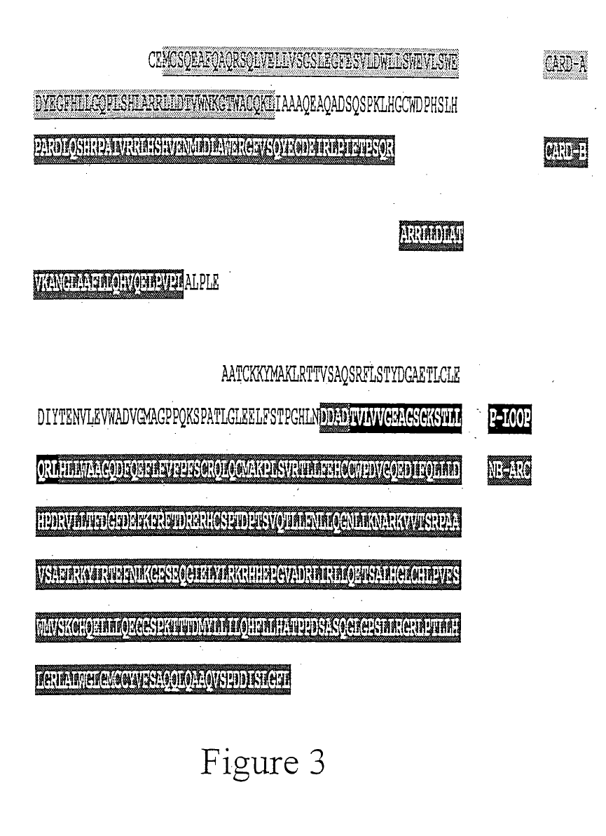

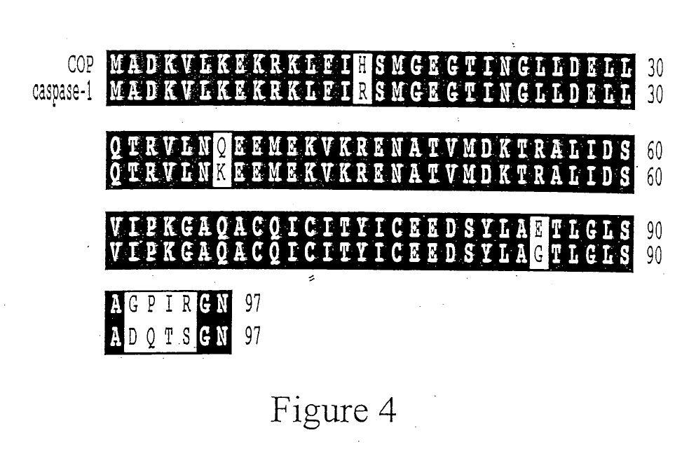

CARD3X contains two CARD domains, a CARD-A and CARD-B domain (see FIG. 3). A NACHT domain was also observed (see FIG. 3). The NACHT is similar to both the CLAN and APAF-1 NACHT domains and to NACHT domains from several plant disease resistance proteins (Aravind et al., Trends Biochem. Sci. 24:47-53 (1999); Young, Curr. Opin. Plant Biol. 4:285-289 (2000)).

An angio-R domain was also identified at amino acids 457-839 of SEQ ID NO:107. An “angio-R” is a new domain that can be defined as a region of a polypeptide chain that bears substantial similarity (e.g. 25, 30, 40% sequence identity) to the 514-reside long protein “angiotensin II/vasopressin receptor” (described in Ruiz-Opazo et al., Nature Med. 1:1074-1081 (1995)). The “angio-R” domain has not been previously described in any protein.

To confirm the predicted sequences, cDNAs were cloned and sequenced. The CARD3X cDNA was cloned using a Rapid-Screen™ Arrayed Placenta cDNA Library Panel from Origene Technologies, Inc. The library cDNAs had been pre-selected for long clones, unidirectionally cloned into the vector pCMV6-XL4, and arrayed in a 96-well format. An initial Master Plate containing 500,000 cDNA clones was screened by PCR, using the forward primer 5′-GAAATGTGCTCGCAGGAGG-3′ (SEQ ID NO:185) and the reverse primer 5′-GATGAGCTTCTGACAGGCCC-3′ (SEQ ID NO:186). A set of 5000 clones that were initially positive by PCR were screened again with the same set of primers. Positive clones were plated on LB/Amp plates, and a further round of single colony PCRs was performed in order to obtain the desired clone.

Three independent clones were sequenced, each of which corresponded to the nucleotide sequence SEQ ID NO:187. The cDNA sequence differed at both the N- and C-terminal ends from the CARD3X sequence predicted from analysis of genomic exons. Nucleotide sequence SEQ ID NO:187 encodes a polypeptide of 795 amino acids (SEQ ID NO:188), followed by a stop codon. A second open reading frame begins after the stop codon, and in the same reading frame, and encodes a polypeptide of 180 amino acids (SEQ ID NO:189). SEQ ID NO:189 contains several leucine rich repeats. SEQ ID NO:197 encodes a polypeptide of 1013 amino acids.

Subsequent to the identification of the two polypeptides encoded by SEQ ID NO:187, a publication reported the cloning of a gene designated Nod2 cloned (Ogura et al., J. Biol. Chem. 276:4812-4818 (2001)). The published Nod2 sequence has additional N-terminal amino acids relative to SEQ ID NO:188 and, instead of the stop codon between the residues that encode SEQ ID NO:188 and SEQ ID NO:189, additional coding sequence is present, which encodes several additional leucine rich repeats. The published Nod2 sequence is 1040 amino acids.

It has been identified that SEQ ID NO:188 is a splice variant form of CARD3X/Nod2 that does not contain an LRR domain. The LRR of Nod2 has been shown to interfere with the ability of the protein to activate NFκB (Ogura et al., supra (2001)). Therefore, SEQ ID NO:188 is likely expressed under physiological conditions in which activation of NFκB is required.

Disclosed herein is that SEQ ID NO:197 is another splice variant form of CARD3X/Nod2 that lacks amino acids 1-27 of SEQ ID NO:189. This CARD3X isoform is likely expressed under physiological conditions in which activation of NFκB is required.

Human CARD3X cDNA sequences were used as a query for BLAST searches of several mouse databases. A genomic sequence, SEQ ID NO:190, was identified. Nucleotides 191-614 of SEQ ID NO:190 are homologous to the ANGIO-R coding region of human CARD3X. Nucleotides 193-612 of SEQ ID NO:191 were predicted to encode SEQ ID NO:191, which is highly homologous to amino acids 214-341 of the ANGIO-R domain of human CARD3X (SEQ ID NO:176).

PCR was then performed on mouse genomic DNA obtained from C57B6 and NIH3T3 cell lines, using the following primers: Forward primer: 5′-CTGCAGAAGGCTGAGCCACACAACCT-3′ (SEQ ID NO:194), Reverse primer: 5′-ACAGAGTTGTAATCCAGCTGTAGGGCCACA-3′ (SEQ ID NO:195). The PCR product so obtained was sequenced (SEQ ID NO:192), and shown to have several nucleotide differences as compared to the corresponding region of SEQ ID NO:190. The predicted amino acid sequence encoded by SEQ ID NO:192 (designated SEQ ID NO:193) had a single amino acid difference in comparison with SEQ ID NO:191.

Both the CARD-A and CARD-B domains are independently cloned into pcDNA3 with epitope tags such as myc or HA, as described above, and binding of the CARD domains is tested with co-immunoprecipitation to test binding of CARD3X CARD domains with other known CARD domains, as described above.

The NACHT domain is cloned into a yeast two-hybrid vector and into pcDNA3 with two alternative epitope tags (e.g., myc and Flag) to determine whether the NACHT domain self-associates in an ATP-dependent manner/P-loop mutation. The P-loop, which binds the gamma phosphate of ATP in the NACHT domain, is mutated to remove a conserved Lys in the consensus P-loop sequence G-S/T-K, where Lys is generally mutated to Met. The NACHT domain is also tested for binding to the NB-domains of other CED-4 like proteins (e.g., apaf1, nod1, nac).

12.0 Characterization of COP-1. Using the amino-acid sequence of the caspase-1 prodomain as a query for BLASTn searches of the public databases, a human EST clone (GenBank accession number AA070591) was identified containing an ORF encoding a 97 amino-acid protein (SEQ ID NO:86) predicted to share 92% sequence identity with the CARD of pro-caspase-1 (SEQ ID NO:87). The predicted protein contains a CARD (residues 1-91), which is followed by 6 amino-acids and then a stop-codon. The CARD region of COP-1 showed 97% identity to the CARD of pro-caspase-1.

To confirm the predicted sequences, cDNAs were amplified from various adult human tissues and sequenced. The sequenced COP-1 cDNA (SEQ ID NO:85) had the same nucleotide sequence as the original EST.

The start codon initiating the ORF in the COP-1 cDNA clones resides in a favorable context for translation, and is preceded by an in-frame stop codon. The 3′-untranslated region contains TAAA and TATA motifs, typical of short-lived mRNAs which are subject to post-transcriptional regulation, and a candidate polyadenylation signal sequence (AATAAA). Thus, this protein contains essentially only a CARD, prompting the moniker CARD Only Protein (COP-1).

To determine the genomic organization of the COP-1 gene, the COP-1 cDNA nucleotide sequence was employed for searches of the High Throughput Genomic Sequence (HTGS) database, resulting in identification of three genomic clones containing the COP-1 gene (GenBank accessions numbers AC027011, AP001153 and AP002787). Comparison of the COP-1 cDNA and genomic DNA sequences suggests a three exon structure, in which only the first two amino-acids are encoded in exon 1 and only the last 5 residues are encoded in exon 3, such that most of the coding regions (including the entire CARD) are derived from exon 2. The introns separating exons 1, 2, and 3 are 631 and 844 bp in length, respectively, containing consensus dinucleotide splice donor (GT) and splice acceptor (AG) motifs.

The COP-1 genomic clones identified in the HTSG database have been mapped to human chromosome 11q22, which is the same chromosomal region where the pro-caspase-1 gene resides, as well as pro-caspase-4, pro-caspase-5, and ICEBERG. To address the genomic localization of COP, pro-caspase-4, pro-caspase-5, and ICEBERG genes in chromosome 11, the public database of Human Genome Project Working Draft (www.genome.cse.ucsc.edu) was searched, and the order of these genes from centromere to telomere was determined to be pro-caspase-4, pro-caspase-5, pro-caspase-1, COP, and ICEBERG. This result suggests that COP-1 is a separate gene, presumably arising from duplication of other homologous genes in this locus.

14.1 COP-1 expression. To study the expression of COP-1, Northern blot analysis was performed using RNA derived from several adult human tissues and a ³²P-labeled COP-1 cDNA probe. Blots containing polyA-selected mRNA from various adult tissues (Clontech, Palo Alto, Calif.) were hybridized using a ³²P-labeled COP-1 cDNA probe. The probe represented a 570 bp length cDNA containing portions of the 5′-untranslated region, the complete ORF, and portions of the 3′-untranslated region of COP. The COP-1 probe (from the EST clone corresponding to AA070591 obtained from the I.M.A.G.E. Consortium (Washington University School of Medicine, St. Louis, Mo.)) was excised from the plasmid by restriction digestion with EcoRI and XhoI, gel-purified, and radiolabeled by the random priming method using [α-³²P] dCTP and a kit from Ambion (Austin, Tex.). After hybridization, heat-denatured probe was annealed for 1 hour at 68° C. with QuickHyb Hybridization Solution (Stratagene, La Jolla, Calif.) and then blots were washed with solutions containing 2×SSC, 0.1% (w/v) SDS (twice each for 15 min at 25° C.) followed by 0.1×SSC, 0.1% (w/v) SDS (twice for 10 min at 40° C.). Bands were visualized by autoradiography.

Hybridizing bands of approximately 0.6 kbp, 1.5 kbp and 2.6 kbp were identified, with the 0.6 kbp band representing the most abundant of these transcripts and presumably corresponding to the fully-spliced COP-1 mRNA. The less abundant larger 1.5 kbp and 2.6 kbp transcripts could represent unspliced precursors. Alternatively, the 2.6 kbp mRNA could represent pro-caspase-1 mRNA, resulting from probe cross-hybridization. The 0.6 kbp COP-1 mRNA was most abundant in spleen, followed by liver, placenta, and peripheral blood leukocytes (PBL). However, most tissues (including heart, muscle, colon, kidney, intestine and lung) were shown to contain at least some detectable 0.6 kbp COP-1 mRNA.

To corroborate the Northern blot analysis, COP-1 mRNA expression in adult human tissues was also examined using RT-PCR and COP-specific primers. cDNA samples derived from multiple human adult tissues (Clontech, Palo Alto, Calif.) were amplified using a set of COP-specific primers (a forward primer 5′-GAAGACAGTTACCTGGCAGA-3′ (SEQ ID NO:147) and a reverse primer 5′-TTGTATTCTGAACATGGCACC-3′ (SEQ ID NO:148)). The resulting PCR products were size-fractionated by electrophoresis in 1.5% agarose gels, then stained with ethidium bromide for UV-photography. In some cases, bands were excised from gels, purified, and sequenced, thus verifying amplification of the correct product by the RT-PCR assay.

RT-PCR analysis showed that COP-1 mRNA was expressed in all tissues analyzed (brain, heart, muscle, colon, spleen, kidney, liver, intestine, placenta, lung and PBL), except thymus. Parallel RT-PCR analysis of β-actin mRNA served as a control. In general, the relative levels of COP-1 mRNA detected by RT-PCR were in agreement with the Northern blot data.

14.2 COP-1 interactions. The prodomain of pro-caspase-1 is required for dimerization and activation of this zymogen. Since the prodomain of COP-1 shares a high-degree of amino-acid sequence identity with the prodomain of caspase-1, the possibility that COP-1 interacts with pro-caspase-1 in co-immunoprecipitation assays was tested. Interactions with several other CARD-containing proteins were also tested, including COP-1 itself, RIP2, Bcl-10, cIAP1, cIAP2 and pro-caspase-9.

For these experiments, the entire open reading frame (ORF) of COP-1 was amplified by PCR using the primers (5′-CCAGAATTCATGGCCGACAAGGTCCTGAAG-3′ (SEQ ID NO:145) (forward) and 5′-CCACTCGAGCTAATTTCCAGGTATCGGACC-3′ (SEQ ID NO:146) (reverse). The COP-1 PCR product was digested with EcoRI/XhoI and ligated into mammalian expression vectors pcDNA3-Myc, pcDNA3-HA and pcDNA3-Flag at the EcoRI/XhoI cloning sites. Plasmids encoding wild-type pro-caspase-1, RIP2, and pro-IL-1β were as described in Thome et al., Curr, Biol. 8:885-888 (1998); Nett-Fiordalisi et al., J. Leukoc. Biol. 58:717-724 (1995); and Wang et al., J. Biol. Chem. 271:20580-20587 (1996).

A pro-caspase-1 Cys 285 Ala mutant was made from wild-type caspase-1 plasmid by site-directed mutagenesis, using a commercially available kit (Stratagene, La Jolla, Calif.) and the primers 5′-GATCATCATCCAGGCCGCCCGTGGTGACAGCCCTGG-3′ (SEQ ID NO:149) and 5′-CCAGGGCTGTCACCACGGGCGGCCTGGATGATGATC-3′ (SEQ ID NO:150). A truncation mutant of pro-caspase-1 in which a stop codon was introduced downstream of the CARD was created by PCR using primers

(SEQ ID NO: 151)

5′-CGGAATTCATGGCCGACAAGGTCCTG-3′
and

(SEQ ID NO: 152)

CGCTCGAGTTAGTCTTGCATATTAAGGTAATTTCCAGA-3′.

Human embryonic kidney 293T cells were cultured at 37° C. in 5% CO₂ in Dulbecco's Modified Eagle's Medium (DMEM) with 10% heat-inactivated fetal bovine serum (FBS). Cells in log phase were transfected in 60 mm diameter dishes with expression plasmids (5 μg total DNA) using Superfect Transfection Reagent (Qiagen, Valencia, Calif.) according to the manufacturer's recommendations. Cells were harvested 2 days later and lysed in ice-cold NP-40 lysis buffer (10 mM HEPES [pH 7.4], 142.5 mM KCl, 0.2% NP-40, 5 mM EGTA), supplemented with 1 mM DTT, 12.5 mM β-glycerophosphate, 1 μM Na₃VO₄, 1 mM PMSF, and 1× protease inhibitor mix (Roche, Indianapolis, Ind.). Cell lysates (0.5 ml) were clarified by centrifugation at 16,000×g for 5 minutes, and subjected to immunoprecipitation using specific antibodies, including anti-Myc antibodies (Santa Cruz Biotechnology, Santa Cruz, Calif.), and anti-Flag antibodies (Sigma, St. Louis, Mo.), in combination with 15 μl Protein A- or G-Sepharose (Zymed, South San Francisco, Calif.).

Immune-complexes were fractionated by sodium dodecyl sulfate-polyacrylamide gel electrophoresis (SDS-PAGE), and transferred to nitrocellulose membranes. The resulting blots were incubated with various antibodies, including anti-HA antibodies (1:1000 v/v; Roche, Indianapolis, Ind.), anti-Myc antibodies (1:100 v/v; Santa Cruz Biotechnology, Santa Cruz, Calif.) and anti-Flag antibodies (1:1000 v/v; Sigma, St. Louis, Mo.), followed by horseradish peroxidase-conjugated secondary antibodies, and detection by an enhanced chemiluminescence (ECL) method (Amersham-Pharmacia, Piscataway, N.J.). Alternatively, lysates were analyzed directly by immunoblotting after normalization for total protein content.

The co-immunoprecipitation results showed that HA-COP-1 co-immunoprecipitated with Myc-COP, indicating that this protein can self-associate. In addition, HA-COP-1 co-immunoprecipitated with Myc-tagged pro-caspase-1 (C285A mutant) as well as with a fragment of pro-caspase-1 containing only its CARD-carrying prodomain. Thus, COP-1 binds pro-caspase-1 through its CARD domain. For these co-immunoprecipitation experiments, the active site cysteine of pro-caspase-1 was mutated to avoid induction of apoptosis, which can occur when over-expressing this protease. Additionally, Myc-COP-1 co-immunoprecipitated with Flag-RIP2. In contrast, COP-1 did not co-immunoprecipitate with the CARD-containing proteins Bcl-10, cIAP1, cIAP2, or pro-caspase-9, thus demonstrating the specificity of these results.

RIP2 has been shown to bind and activate caspase-1 through the interaction of their CARDS, resulting in oligomerization of pro-caspase-1 and its activation via the “induced proximity” mechanism. The data demonstrating that COP-1 binds to both pro-caspase-1 and RIP2 therefore suggested that COP-1 might function as a modulator of RIP2-induced pro-caspase-1 oligomerization.

To test this hypothesis, experiments were performed in which 293T cells were transiently transfected with expression plasmids encoding Myc-tagged pro-caspase-1 (C285A mutant) and HA-tagged pro-caspase-1 (C285A mutant), with or without Flag-tagged RIP2 and COP, after which Myc-pro-caspase-1 and HA-pro-caspase-1 association was monitored by co-immunoprecipitation assays.

As determined by this co-immunoprecipitation assay, pro-caspase-1 self-associated and this was enhanced by co-expression of RIP2. However, when COP-1 was also co-expressed, this RIP2-mediated effect on pro-caspase-1 self-association was negated. These findings suggested the possibility of a competitive mechanism, in which COP-1 competes with RIP2 for binding to pro-caspase-1. To test this hypothesis, therefore, transfection experiments were preformed in which Flag-RIP2 and Myc-tagged pro-caspase-1 (C285A mutant) were expressed in 293T cells in the presence of increasing amounts of HA-tagged COP-1. The effects of COP-1 on association of RIP2 with pro-caspase-1 were then evaulated by co-immunoprecipitation assays in which immunoprecipitations were performed using anti-Flag antibody to recover Flag-RIP2 protein and the resulting immune-complexes were analyzed by SDS-PAGE/immunoblotting using anti-Myc antibody to detect associated Myc-pro-caspase-1.

The results from these experiments indicated that COP-1 inhibited association of pro-caspase-1 with RIP2 in a dose-dependent manner. Immunoblot analysis of lysates from these same cells demonstrated that COP-1 did not affect the total levels of pro-caspase-1 or RIP2, but rather just their association. These results therefore confirm that COP-1 can interfere with binding of pro-caspase-1 to RIP2.

14.3 COP-1 inhibition of caspase-1-mediated activation of pro-IL-1β. Active caspase-1 cleaves pro-IL-1β, resulting in the generation of bioactive IL-1β which is secreted from cells. It was hypothesized that COP-1 could suppress caspase-1-induced pro-IL-1β processing and thus reduce secretion of IL-1β.

To test this hypothesis, COS-7, 293T, or 293HEK cells were co-transfected in 12 well (22 mm in diameter) plates using Lipofectamine Plus Reagent (GIBCO BRL, Grand Island, N.Y.) with plasmids encoding mouse pro-IL-1β, human caspase-1, RIP2, or COP-1, in various amounts (total DNA=2.0 μg). At 1 day after transfection, supernatants were collected and stored at −80° C. or used immediately to quantify secretion of mature murine IL-1β into the culture medium by an ELISA assay, according to the manufacturer's protocol (R&D systems, Minneapolis, Minn.).

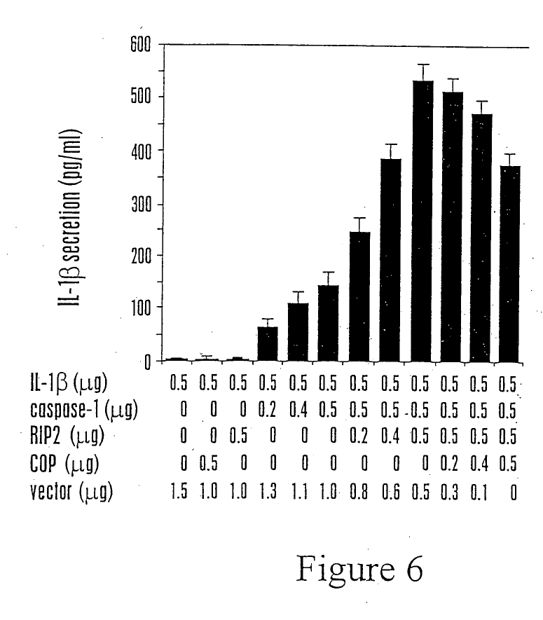

Co-expression of pro-caspase-1 and pro-IL-1β in COS-7 cells resulted in secretion of mature IL-1β ranging from 80 pg/ml to 250 pg/ml, which was proportional to the amount of pro-caspase-1 plasmid used (FIG. 17). This IL-1β secretion was enhanced by co-expression of RIP2 plasmid. In contrast, expression of COP-1 together with pro-caspase-1, pro-IL-1β, and RIP2 resulted in a dose-dependent decrease in the amount of mature IL-1β secretion, proportional to the amount of COP-1-encoding plasmid used (FIG. 6). Similar results were obtained using 293T or 293HEK cells. These results indicate that COP-1 is capable of suppressing the caspase-1-mediated secretion of IL-1β.

15.0 Identification of COP-2. A human CARD-containing proteins, designated COP-2, for CARD-only protein 2, was identified and the gene and cDNA cloned. The predicted protein of COP-2 has high sequence similarity to the CARD-domain of human caspase-1. For COP-2, two primers based on the caspase-15 genomic sequence were designed, one in the middle of the CARD domain (5′-aagaagagacggctgcttatcaat-3′; SEQ ID NO:104) and the other in the catalytic domain (5′-ccacagcaggcctcgaagatgatc-3′; SEQ ID NO:105). RT-RTR was performed, and a single band was observed, although the band size was smaller than expected for caspase-15. The PCR product was sequenced, and it was found that two exons were deleted and the catalytic domain was directly connected to the CARD domain. However, due to a frameshift, a stop codon occurs just after the CARD domain, resulting in truncated protein and no translation of the catalytic domain.

To clone the N-terminal region, a primer (5′-atgatcctcctgaagaagag-3′; SEQ ID NO:106) was designed with the genomic sequence in the most N-terminal portion of the CARD domain including ATG. RT-PCR was performed, and the PCR product was sequenced and found to be the same as in the genomic DNA. A merged construct containing both the N-terminal fragment and the CARD domain sequence was made by PCR.

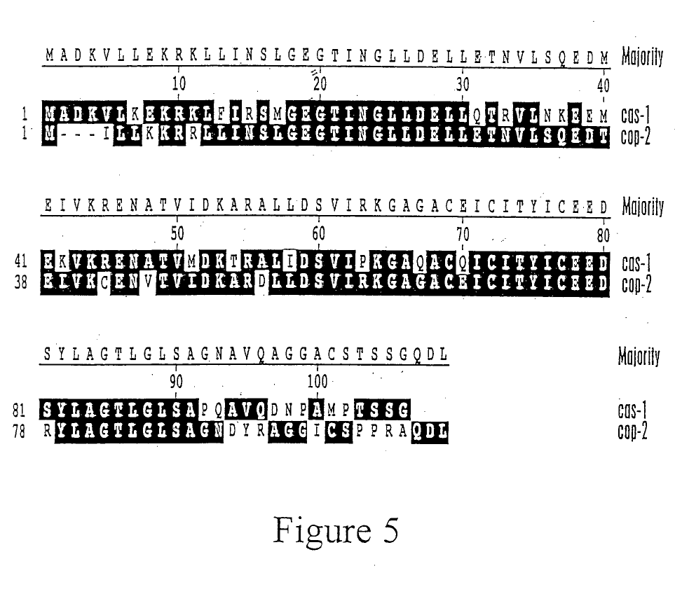

The COP-2 cDNA sequence identified contained 321 nucleotides (SEQ ID NO:89), and the deduced amino acid sequence (SEQ ID NO:90) had a high level of identity with caspase-1. An alignment of COP-2 (SEQ ID NO:90) and caspase-1 (SEQ ID NO:87) is shown in FIG. 5, with the consensus sequence (SEQ ID NO:91) shown above the aligned sequences. The amino acids shaded in black are identical. The stipled shading represents a match within 3 distance units. COP-2 is encoded by the caspase-15 gene (FIG. 3), but COP-2 is a CARD only protein that lacks the caspase catalytic domain.

COP-2 cDNA encodes a polypeptide with downstream termination codons, which result in shorter proteins containing a CARD domain without associated catalytic protease domains. COP-2 is therefore expected to function as trans-dominant inhibitor that likely prevents caspase activation by binding to the CARD-domains (pro-domains) in pro-enzymes such as pro-caspase-1.

COP-2 polypeptide is expected to function as A regulator of caspase-1 activation by enhancing or suppressing the activation of caspase-1. COP-2 binding activity is tested, for example, by making epitope tagged fusions with COP-2 and caspase-1 and co-immunoprecipitating to determine binding interactions with caspase-1. Antibodies specific for COP-2 are also made.

The effect of COP-2 on caspase-1 proteolytic activity is also tested. Methods for measuring caspase activity are well known (see, for example, Thornberry, Nature 356:768-774 (1992); Thornberry and Molineaux, Protein Science 4:3-12 (1995); Rano et al., Chem. Biol. 4:149-155 (1997); Fletcher et al., J. Interferon Cytokine Res. 15:243-248 (1995)), and are also described above.

16.0 Cloning of CARD3X-2 (Short CARD3X Isoform)

CARD3X-2 was cloned by 5′ and 3′ RACE PCR from cDNA generated from the THP-1 cell line using the SMART RACE cDNA amplification kit (Clontech). The following primers were used for amplification: (5′ RACE) 5′-CAAGATCAAGCAGCCTTCTTGCCCTCTGG-3′ (SEQ ID NO: 198); (3′ RACE) 5′-CATCCAGGCCTCGGAGGGAAAGGACAGCAG-3′ (SEQ ID NO:199). Bands were excised from an agarose gel, purified, and subjected to nested PCR to confirm CARD3X specificity. 5′ and 3′ RACE products were then cloned into the TOPO cloning vector (Invitrogen, Carlsbad, Calif.) and multiple clones were sequenced for each. Clones of the 3′ end of CARD3X-2 were consistent with published CARD3X sequences. Clones of the 5′ end revealed two previously unreported alternatively spliced exons that altered the start of the ORF of the published CARD3X sequence by 78 base pairs (26 amino acid residues). The following primers were used to amplify the full-length Nod2 cDNA from normal human monocyte cDNA: (FW) 5′-CGGAATTCATGTGCTCGCAGGAGGCTTTTC-3′ (SEQ ID NO:200); (REV) 5′-CAAGTTCAGCCTTAGGCAGGAC-3′ (SEQ ID NO:201). Products were again excised from an agarose gel and cloned into the TOPO cloning vector. Multiple clones were sequence-verified and the open reading frame (ORF) encoding full-length CARD3X-2 was cloned into the pcDNA.3(−)/myc-his₆(A) plasmid. The plasmids pcDNA3/myc-NAC, pcDNA3/myc-PAN2, and pcI-Flag-Nod1 were previously described (Chu et al., J. Biol. Chem. 276:9239-9249 (2001); Fiorention et al., J. Biol. Chem. 277:35333-35340 (2002) and Inohara et al., J. Biol. Chem. 274:14560-15467 (1999))

17.0 Association of NACHT Domain Containing Polypeptides

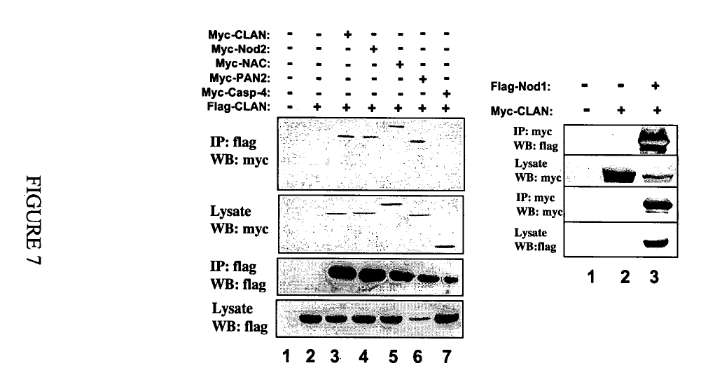

To assess associations between various NACHT-containing proteins, HEK293T cells were transiently transfected with expression plasmids encoding full-length, epitope-tagged CLANA, which contains CARD, NACHT, and LRR domains (Damiano et al., Genomics 75:77-83 (2001)), along with plasmids coding for other epitope-tagged NACHT-containing proteins or various control proteins. Co-immunoprecipitation studies demonstrated that CLANA associates with itself as well as with Nod2 (CARD3X), PAN2 (NALP4), NAC (NALP1/Defcap), when co-expressed in 293T cells (see FIG. 7). CLAN associated more weakly with Nod1 (CARD4) but did not associate with other CARD-containing proteins that lack a NACHT domain, such as pro-caspase-4 and pro-caspase-9.

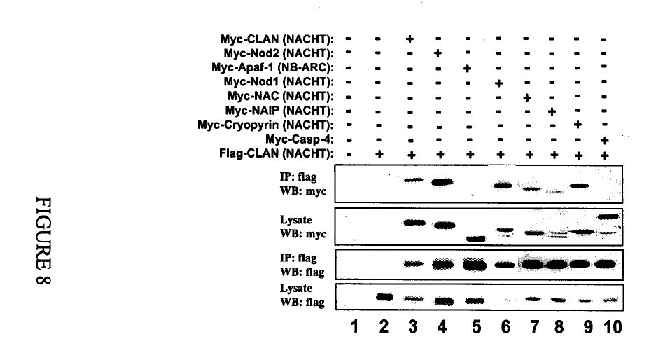

To determine if the NACHT domain by itself is capable of mediating these heterologous protein interactions, an expression plasmid encoding the epitope-tagged NACHT domain of CLAN was co-expressed with various other epitope-tagged NACHT domains in 293T cells. Following immunoprecipitation and immunoblotting, the NACHT domain of CLAN was found to associate with itself as well as with the NACHT domains of Nod1, CARD3X (Nod2), NAC, NAIP, PAN2, and Cryopyrin (see FIG. 8). In contrast, the NACHT domain of CLAN did not bind to pro-caspase-4 and bound weakly to the NACHT domain of Apaf1.

17.1 CLAN's NACHT Domain is Associated with a Large Protein Complex in Cells

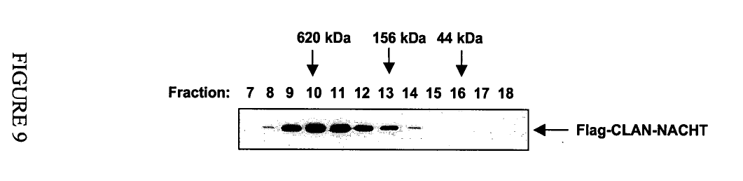

To assess whether CLAN's NACHT domain exists as a monomer or as part of a larger complex within the cell, 293T cells were transfected with flag-tagged CLAN-NACHT and lysed 24 hours later. Lysates were fractionated by gel-filtration using a Superdex-200 column, fractions were collected and analyzed by immunoprecipitation with agarose-conjugated anti-flag antibodies. Western blotting demonstrated that the NACHT domain associated with protein complexes of sizes primarily between 156 and 620 kDa (see FIG. 9). No evidence that CLAN-NACHT exists as a monomer was obtained in these assays, suggesting that the NACHT domain of CLAN spontaneously either self-associates or binds other proteins present endogenously in HEK293T lysates.

17.2 CLAN Inhibits Nod-Mediated NF-κB Activation.

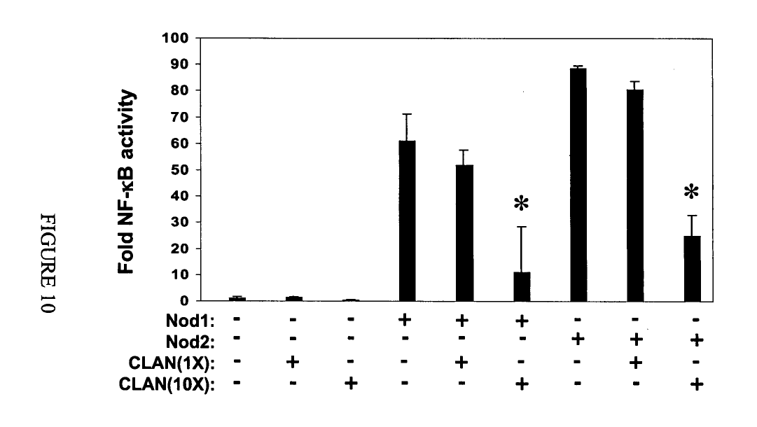

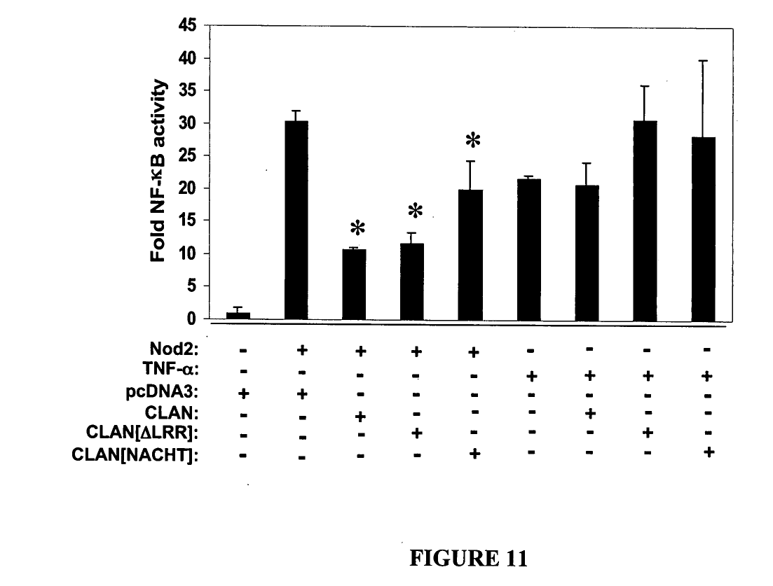

Having shown that CLAN interacts with other NACHT-family proteins, it was next determined that CLAN affects the functions of these proteins. Nod1 and CARD3X (Nod2) are known to bind CARDIAK/RIP2/RICK via their CARD domains, subsequently leading to IKK activation, IκBα degradation, and nuclear translocation of NF-κB transcription factors (Inohara et al., J. Biol. Chem. 275:27823-27831 (2000) and Inohara, supra, 1999). An NF-κB-receptor gene assay was used to assess the effects of CLAN on Nod-mediated NF-κB activation in HEK2932T cells. Over-expression of Nod1 or CARD3X (Nod2) induced NF-κB activity in these transient transfections (see FIG. 10). When CLAN was co-expressed with full-length Nod1 or CARD3X (Nod2), NF-κB activity was significantly reduced. Western blotting using whole cell lysates demonstrated equivalent levels of CARD3X (Nod2) protein between samples.

To map the domain in CLAN involved in suppression of Nod2-induced NF-κB, we contrasted full-length CLAN with a fragment of CLAN lacking the LRR, and with a fragment comprising only the NACHT domain. These experiments show that the NACHT domain of CLAN is sufficient to suppress NF-κB activation by CARD3X (Nod2) (see FIG. 10). Furthermore, this inhibition is specific, because CLAN or the NACHT domain of CLAN did not interfere with NF-κB activity induced by other stimuli such as TNF-α.

17.3 Effects of CLAN on Nod-Mediated IL-1β Secretion

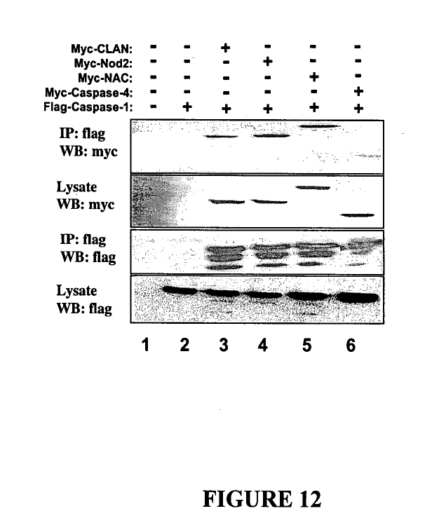

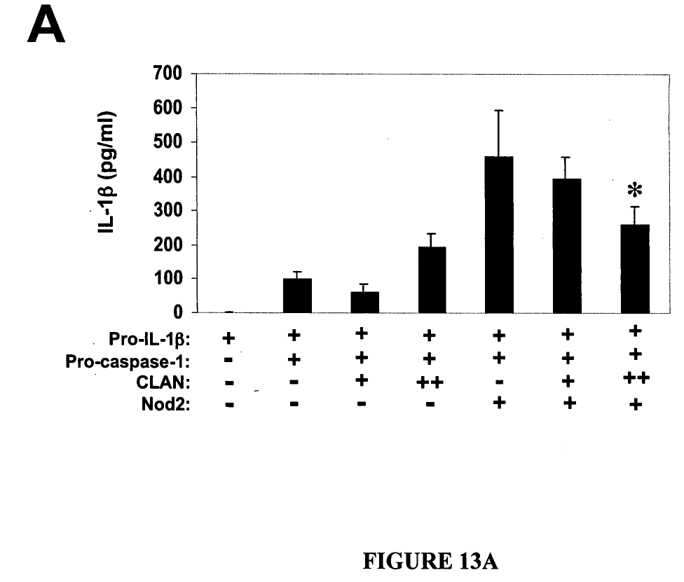

Nod1 has been shown to associate with pro-caspase-1 and induce its proteolytic activation in over-expression studies (Yoo et al., Biochem. Biophys. Res. Commun. 299:652-658 (2002)). Caspase-1 cleaves pro-Interleukin-1β (pro-IL-1β), generating the mature form of this cytokine which is subsequently secreted from cells. As is shown in FIG. 12, CARD3X (Nod2), CLAN, and NAC also associate with pro-caspase-1 in transient transfection/co-immunoprecipitation studies of epitope-tagged proteins in 293T cells.

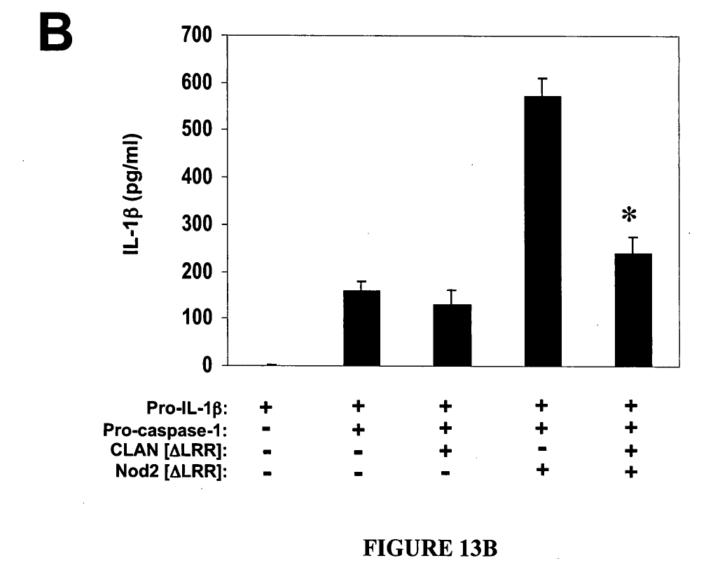

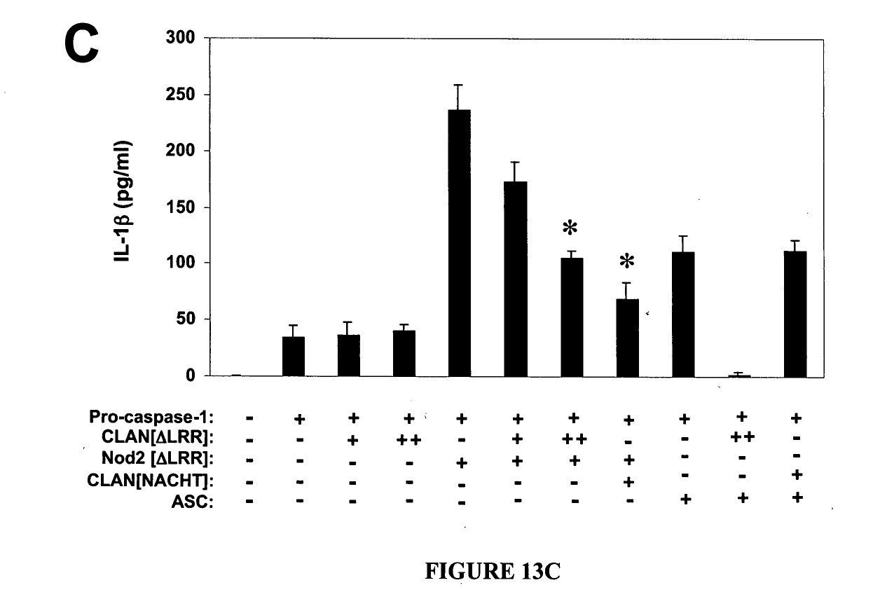

The effects of CLAN on the caspase-1 dependent production of secreted IL-1β was also tested. For initial experiments, HEK293T cells were co-transfected with plasmids encoding full-length pro-caspase-1, CARD3X (Nod2), CLAN, or combinations of these proteins. The combination of pro-caspase-1 and CARD3X (Nod2) resulted in substantial amounts of IL-1β secretion, while CLAN co-transfection with pro-caspase-1 had relatively little effect (see FIG. 13A). In contrast, co-expressing CLAN with pro-caspase-1 and CARD3X (Nod2) resulted in less IL-1β secretion compared to pro-caspase-1 and CARD3X (Nod2) alone.

Expression plasmids encoding CARD3X (Nod2) lacking the LRR domains were used in experiments to determine if this form of CARD3X can enhance caspase-1-mediated production of IL-1β. The LRR domain is known to auto-repress NACHT-family protein activity unless certain bacteria-derived ligands are present (Poyet et al., J. Biol. Chem. 276:28309-28313 (2001) and Martinon et al., Mol. Cell. 10:417-426 (2002)), analogous to the WD-40 repeat region of Apaf1 which represses its own activation in the absence of cytochrome c (Hu et al., J. Biol. Chem. 273:33489-33494 (1998)). As seen in FIG. 13B, expression of CARD3X[ΔLRR] enhanced caspase-1-mediated production of IL-1β in culture supernatant. Since CLAN interacts with both pro-caspase-1 and the Nod proteins, the effect of CLAN in this IL-1β secretion assay was examined. The levels of CLAN or CLAN[ΔLRR], a mutant of CLAN reported to be constitutively active (Poyet et al., supra, 2001), expressed in these assays did not significantly increase IL-1β secretion. Instead, CLAN inhibited the CARD3X-mediated secretion of IL-1β (see FIG. 13B). Additionally, the NACHT domain of CLAN by itself was sufficient to inhibit Nod2-mediated IL-1β production. Similar observations were made when full-length CLAN, CLAN[ΔLRR], or the NACHT domain of CLAN was co-expressed with Nod1.

To determine the specificity of the effects of the NACHT domain of CLAN on CARD3X-mediated IL-1β production, its effects on CARD2X were compared with ASC, another caspase-1-activating protein. As shown in FIG. 13C, the NACHT domain of CLAN as well as a NACHT-containing fragment of CLAN lacking the LRRs (CLAN[ΔLRR]) suppressed IL-1β secretion induced by transfection of cells with CARD3X in combination with pro-caspase-1. In contrast, the NACHT domain of CLAN failed to suppress IL-1β secretion induced by transfecting cells with the combination of ASC and pro-caspase-1 (see FIG. 13C). Interestingly, while the NACHT domain of CLAN did not suppress ASC-induced IL-1β secretion, the CLAN[ΔLRR] protein did, presumably because the CARD domain of CLAN is known to bind pro-caspase-1 and thus competes with ASC for access to pro-caspase-1

17.4 Co-Immunoprecipitation Assays.

HEK293T cells (0.5×10⁶) were seeded into 6 well plates and grown overnight. The following day, Lipofectamine Plus was used to transfect 2 μg of various expression plasmids according to the manufacturer's recommended protocol. After 24 hours, cells were recovered and lysed in isotonic co-immunoprecipitation buffer (142 mM KCl, 0.2% NP-40, 5 mM mgCl₂, 10 mM HEPES, 0.5 mM eGTA, 12.5 mM β-glycerophosphate, 2 mM NaF, 1 mM Na₃VO₄, 1 mM PMSF and 1× protease inhibitor mix from Roche). Lysates were clarified by centrifugation and subjected to immunoprecipitation for 2-24 hours at 4° C. using agarose-conjugated anti-myc antibody (Santa Cruz Biotechnology, Inc., Santa Cruz, Calif.), anti-flag antibody (Sigma), or non-specific antibodies coupled to protein-G-agarose. Immune-complexes were washed extensively with lysis buffer, boiled in Laemmli buffer, and separated by SDS-PAGE using 8 or 10% gels. Proteins were then transferred to nitrocellulose membranes and detected by immunoblotting using anti-c-myc (Santa Cruz Biotechnology, Inc., Santa Cruz, Calif.) or anti-flag (Sigma) antibodies conjugated to horse radish peroxidase (HRPase), followed by visualization using an enhanced chemiluminescence (ECL) method (Amersham).

17.5 NF-κB Luciferase Reporter Assays.

Cells were seeded at 1×10⁵ per well in 24 well plates. The following day, cells were transfected with 100 ng pNF-κB-luc, 50 ng pTK-RL (Stratagene, La Jolla, Calif.), and various expression plasmids (as indicated in figure legends) using Superfect (Qiagen). Total DNA (0.85 μg) was kept constant using empty pcDNA3/myc plasmid. After 24 hours, cells were lysed and activity from firefly and renilla luciferases was assayed using a Dual-Luciferase Reporter System (Promega, Madison, Wis.) and a luminometer.

17.6 Interleukin-1β Detection.

Cells were seeded at 1×10⁵ per well of 24 well plates. The following day, cells were transfected using Superfect according to the manufacturer's protocol. Amounts of each plasmid used are indicated in figure legends, maintaining total DNA constant using empty pcDNA3/myc plasmid. At 24 hours post-transfection, supernatants were collected, clarified by centrifugation, and assayed for the presence of mature IL-1β using a murine IL-1β ELISA (R&D Systems, Minneapolis, Minn.).

17.7 Gel Filtration Analysis

Cells were seeded at 8×10⁶ per 150 mm tissue culture dish and transfected 24 hours later using Lipofectamine Plus according to the recommended protocol. After 24 hours, cells were recovered and lysed in buffer containing 100 mM NaCl in combination with 0.2% NP-40, 1 mM EDTA, 1 mM DTT, 0.5 mM MgCl₂, and 0.5 mM EGTA. Lysates were dialyzed overnight and then separated using a Superdex-200 column. Fractions were collected and subjected to anti-flag immunoprecipitation, then proteins were separated by SDS-PAGE (10% gels) and detected as above.

Although the invention has been described with reference to the examples above, it should be understood that various modifications can be made without departing from the spirit of the invention.

All journal article, reference and patent citations provided above, in parentheses or otherwise, whether previously stated or not, are incorporated herein by reference in their entirety.

Although the invention has been described with reference to the examples provided above, it should be understood that various modifications can be made without departing from the spirit of the invention.

1. An isolated nucleic acid molecule encoding a CARD3X-2 polypeptide comprising the amino acid sequence set forth as SEQ ID NO:197, or a domain of said polypeptide selected from a CARD domain, NACHT domain, and LRR domain.

2. The nucleic acid molecule of claim 1, which encodes a CARD3X-2 polypeptide comprising the amino acid sequence set forth as SEQ ID NO:197.

3. The nucleic acid molecule of claim 1, wherein the nucleotide sequence of said nucleic acid molecule comprises SEQ ID NO:196.

4. The nucleic acid molecule of claim 1, wherein said nucleic acid molecule is cDNA.

5. A vector containing the nucleic acid molecule of claim 1.

6. Recombinant cells containing the nucleic acid molecule of claim 1.

7. An oligonucleotide comprising at least 15 contiguous nucleotides of the nucleic acid molecule of claim 3, or the complement thereof.

8. An oligonucleotide according to claim 7, wherein said oligonucleotide is labeled with a detectable marker.

9. An isolated CARD3X-2 polypeptide, comprising the amino acid sequence set forth as SEQ ID NO:197, or a domain of said polypeptide selected from a CARD domain, NACHT domain, and LRR domain.

10. The isolated CARD3X-2 polypeptide of claim 9, comprising the amino acid sequence set forth as SEQ ID NO:197.

11. A method of producing a CARD3X-2 polypeptide comprising expressing the cDNA of claim 4 in vitro or in a cell under conditions suitable for expression of said polypeptide.

12. An isolated anti-CARD3X-2 antibody having specific reactivity with the CARD3X-2 polypeptide of claim 9.

13. The antibody of claim 12, wherein said antibody is a monoclonal antibody.

14. A cell line producing the monoclonal antibody of claim 13.

15. The antibody of claim 12, wherein said antibody is a polyclonal antibody.

16. A method for detecting the presence of a CARD3X-2 polypeptide in a sample, comprising contacting a test sample with an antibody according to claim 12 or a recombinant phage, detecting the presence of an antibody:CARD3X-2 complex or recombinant phage:CARD3X-2 complex, and thereby detecting the presence of a CARD3X-2 polypeptide in said sample.

17. A method of identifying a CARD3X-2 binding molecule comprising:
(a) contacting the CARD3X-2 polypeptide of claim 9 with a candidate CARD3X-2 binding molecule;
(b) detecting association of CARD3X-2 polypeptide with said CARD3X-2 binding molecule.

18. The method of claim 17, wherein the CARD3X-2 binding molecule is a CARD3X-2-associated polypeptide.

19. The method of claim 17, wherein the CARD3X-2 binding molecule is a small molecule.

20. A method of identifying an effective agent that alters association of a NACHT-containing polypeptide with a NACHT-associated polypeptide (NAP), comprising the steps of:
(a) contacting a NACHT-containing polypeptide selected from SEQ ID NOS:188, 189 and 197, and said NAP with an agent suspected of being able to alter the association of said NACHT-containing polypeptide and said NAP, under conditions that allow association between said NACHT-containing polypeptide and said NAP; and
(b) detecting the altered association of said NACHT-containing polypeptide and said NAP, wherein said altered association identifies an effective agent.

21. The method of claim 20, wherein said NAP is selected from CARD3X, CARD3X-2, Nod1, NAC, PAN2, NAIP and cyropyrin.

22. A CARD3X-2 mRNA targeting molecule, comprising a molecule selected from an anti-sense oligonucleotide, a ribozyme and an si RNA, wherein said molecule binds selectively to an mRNA corresponding to the nucleotide sequence referenced as SEQ ID NO:196, or a portion of said nucleotide sequence.
